SEQUENCE LISTING (54 sequences)

SEQ ID NO: 1 - DNA, 804 bp, Homo sapiens
atgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtga

SEQ ID NO: 2 - protein, 267 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 3 - DNA, 559 bp, Gorilla gorilla
ctcggccgagactcccatgttcccgggcaatggcgacgagcagcctctgaccgagaacccccggaagtacgtcatgggccacttccgctgggaccgattcggccgccgcaacagcagcagcagcagcggcagcggcgcagggcagaagcgcgaggatgtctcagcgggcgaagaccgcggcccgctgcctgagggcggccccgagccccgcagtgatggtgccaagccgggcccgcgcgagggcaagcgctcctactccatggagcacttccgctggggcaagccggtgggcaagaagcggcgcccggtgaaggtgtaccctaacggcgccgaggacgagtcggccgaggccttccccctggagttcaagagggagctgactggccagcgaccccgggagggagatggccccgacggccctgccgatgacggcgccggggcccaggccgacctggagcatagcctgctggtggcggccgagaagaaggacgagggcccctacgggatggagcacttccgctggggcagcccgcccaaggacaagcgctacggcggtttc

SEQ ID NO: 4 - protein, 186 aa, Gorilla gorilla
SAETPMFPGNGDEQPLTENPRKYVMGHFRWDRFGRRNSSSSSGSGAGQKREDVSAGEDRGPLPEGGPEPRSDGAKPGPREGKRSYSMEHFRWGKPVGKKRRPVKVYPNGAEDESAEAFPLEFKRELTGQRPREGDGPDGPADDGAGAQADLEHSLLVAAEKKDEGPYGMEHFRWGSPPKDKRYGGF

SEQ ID NO: 5 - DNA, 795 bp, Macaca nemestrina
atgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagcccgacctttcggccgagactccggtgtttccgggcaatggcgacgagcagcctctgaccgagaacccccggaagtacgtcatgggccacttccgctgggaccgattcggccgccgcaacagtagcagcggcagcgcgcaccagaagcgcgaggacgtcgcggctggcgaagaccgcggcctgctacctgagggtggccccgagccccgtggcgatggcgccgggccgggcccgcgcgagggcaagcgctcctactccatggagcacttccgctggggcaagccggtgggcaagaagcggcgcccggtgaaggtgtaccccaatggcgccgaggacgagtcggccgaggccttccccctggagttcaagagggagctgaccggccagcggccccgggcgggggatggccccgatggccctgccgacgacggcgcggggccccgggccgacctggagcacagcctgctggtggcggccgagaagaaggatgagggcccctacaggatggagcacttccgctggggcagcccgcccaaggacaagcgctacggcggcttcatgacctccgagaagagccagactcccctggtgacactgttcaaaaacgccatcatcaagaacgcctacaagaagggccagtga

SEQ ID NO: 6 - protein, 264 aa, Macaca nemestrina
MPRSCCSRSGALLLALLLQASMEVRGWCLESSQCQDLTTESNLLECIRACKPDLSAETPVFPGNGDEQPLTENPRKYVMGHFRWDRFGRRNSSSGSAHQKREDVAAGEDRGLLPEGGPEPRGDGAGPGPREGKRSYSMEHFRWGKPVGKKRRPVKVYPNGAEDESAEAFPLEFKRELTGQRPRAGDGPDGPADDGAGPRADLEHSLLVAAEKKDEGPYRMEHFRWGSPPKDKRYGGFMTSEKSQTPLVTLFKNAIIKNAYKKGQ

SEQ ID NO: 7 - DNA, 496 bp, Pongo pygmaeus
gaagtacgtcatgggccacttccgctgggaccgatttggccgccgcaacagcagcagcggcagcggtagcggcgcagggcagaagcgcgaggacgtcgcagcgggcgaagaccgcggcccactgcctgagggcggccccgagccccgcagcgatggcgccgagccgggcccgcgcgagggcaagcgctcctactccatggagcacttccgctggggcaagccggtgggcaagaagcggcgcccggtgaaggtgtaccccaacggcgccgaggacgagtcggccgaggccttccccctggagttcaagagggagccgaccggccagcggctccgggagggagatggccccgacggccctgccgatgacggcgccggggcccgggccgacctggagcacaacctgctggtggcggccgagaagaaggacgagggcccctacaggatggagcacttccgctggggcagcccgcccaaggacaagcgctacggcggtttc

SEQ ID NO: 8 - protein, 165 aa, Pongo pygmaeus
KYVMGHFRWDRFGRRNSSSGSGSGAGQKREDVAAGEDRGPLPEGGPEPRSDGAEPGPREGKRSYSMEHFRWGKPVGKKRRPVKVYPNGAEDESAEAFPLEFKREPTGQRLREGDGPDGPADDGAGARADLEHNLLVAAEKKDEGPYRMEHFRWGSPPKDKRYGGF

SEQ ID NO: 9 - DNA, 804 bp, Sus scroffa
atgccgagattgtgcggcagtcgctcgggggccctgctgctgaccttgctgctccaggcctccatgggagtgcgcggctggtgcttggagagcagccagtgtcaggacctctccacggaaagtaacttgttggcgtgcatccgggcctgcaaaccagatctctctgcggagacgcccgtgtttcccggcaacggcgacgcgcaaccgctgaccgagaacccccggaagtacgtcatgggccacttccgctgggaccgcttcggccgccggaatggcagcagcagcggcggcggtggcggtggcggcggcgcgggccagaagcgcgaggaggaggaggtggcggcgggcgaaggccccgggccccgcggagatggcgtcgcgccgggcccgcgccaggacaagcgctcctactccatggagcacttccgctggggcaagcccgtgggcaagaagcggcgcccggtgaaggtgtatcccaacggcgccgaggacgagttggccgaggccttccccctcgagttcaggagggagctggccggggcgccccccgagccggcacgggaccccgaggccccggccgagggcgcggccgcccgggccgagctggagtacgggctggtggccgaggccgaggcggccgagaagaaggacgaagggccctataagatggagcacttccgctggggcagcccgcccaaggacaagcgctacggcggcttcatgacctccgagaagagccagacgcccctggtcacgctgttcaaaaacgccatcgtcaagaacgcccacaagaagggccagtga

SEQ ID NO: 10 - protein, 267 aa, Sus scroffa
MPRLCGSRSGALLLTLLLQASMGVRGWCLESSQCQDLSTESNLLACIRACKPDLSAETPVFPGNGDAQPLTENPRKYVMGHFRWDRFGRRNGSSSGGGGGGGGAGQKREEEEVAAGEGPGPRGDGVAPGPRQDKRSYSMEHFRWGKPVGKKRRPVKVYPNGAEDELAEAFPLEFRRELAGAPPEPARDPEAPAEGAAARAELEYGLVAEAEAAEKKDEGPYKMEHFRWGSPPKDKRYGGFMTSEKSQTPLVTLFKNAIVKNAHKKGQ

SEQ ID NO: 11 - DNA, 798 bp, Bos taurus
atgccgagactgtgcagcagtcgttcggccgccctgctgctggccttgctgcttcaggcctccatggaagtgcgtggttggtgcctggagagcagccagtgtcaggacctcaccacggaaagtaacctgctggcgtgcatccgggcctgcaagcccgacctctccgccgagacgccggtgttccccggcaacggcgatgagcagccgctgactgagaacccccggaagtacgtcatgggccatttccgctgggaccgcttcggccgtcggaatggtagcagcagcagcggagttgggggcgcggcccagaagcgcgaggaggaagtggcggtgggcgaaggccccgggccccgcggcgatgacgccgagacgggtccgcgcgaggacaagcgttcttactccatggaacacttcccctggggcaagccggtgggcaagaagcggcgcccggtgaaggtgtaccccaacggcgccgaggacgagtcggcccaggcctttcccctcgaattcaagagggagctgaccggggagaggctcgagcaggcgcgcggccccgaggcccaggctgagagtgcggccgcccggcctgagctggagtatggcctggtggcggaggcggaggctgaggcggccgagaagaaggactcggggccctataagatggaacacttccgctggggcagcccgcccaaggacaagcgctacggcgggttcatgacctccgagaagagccaaacgccccttgtcacgctgttcaaaaacgccatcatcaagaacgcccacaagaagggccagtga

SEQ ID NO: 12 - protein, 265 aa, Bos taurus
MPRLCSSRSAALLLALLLQASMEVRGWCLESSQCQDLTTESNLLACIRACKPDLSAETPVFPGNGDEQPLTENPRKYVMGHFRWDRFGRRNGSSSSGVGGAAQKREEEVAVGEGPGPRGDDAETGPREDKRSYSMEHFPWGKPVGKKRRPVKVYPNGAEDESAQAFPLEFKRELTGERLEQARGPEAQAESAAARPELEYGLVAEAEAEAAEKKDSGPYKMEHFRWGSPPKDKRYGGFMTSEKSQTPLVTLFKNAIIKNAHKKGQ

SEQ ID NO: 13 - DNA, 663 bp, Canis familiaris
gacctcaccacggaaagtaacctgctggcgtgcatccgggcctgcaagcccgacctctccgccgagacgcccgtgctccccggcaacggcgacgagcagccgctggctgagaacccccggaagtacgtcatgggccacttccgctgggaccggtttggccgccgcaatggcagcgcgggccagaagcgcgaggaagaagaggtggcggcgggcggaggccgcgccccgctgcccgcgggcggcccggggccccgcggcgacggtggcgagctcggcctgcaagagggcaagcgctcctactccatggagcacttccgctggggcaagccggtgggcaagaagcggcgcccggtgaaggtgtaccccaacggcgctgaggacgagtcggccgaggccttccccgtcgagttcaagagggagctggccgggcagcggctggagccggcgctcggccccgagggcccggccgcgggcgtggcggcgctggccgacctggagtacggcctggtggcggaggccggggcggccgagaagaaggacgacgggccctacaagatggagcacttccgctggggcagcccgcccaaggacaagcgctacgtcggcttcatgagctcggagaggagccagacgcccctggtgacgctgttcaaaaacgccatc

SEQ ID NO: 14 - protein, 221 aa, Canis familiaris
DLTTESNLLACIRACKPDLSAETPVLPGNGDEQPLAENPRKYVMGHFRWDRFGRRNGSAGQKREEEEVAAGGGRAPLPAGGPGPRGDGGELGLQEGKRSYSMEHFRWGKPVGKKRRPVKVYPNGAEDESAEAFPVEFKRELAGQRLEPALGPEGPAAGVAALADLEYGLVAEAGAAEKKDDGPYKMEHFRWGSPPKDKRYVGFMSSERSQTPLVTLFKNAI

SEQ ID NO: 15 - DNA, 771 bp, Cavia porcellus
atgccgagatcgtgctacagccgctcggggaccctgctgctggccttgctgcttcagatctccatggaagtgcggggctggtgcctggagagcagccagtgtcaggacctcaccacggagagacacctgctggagtgcctccgggcctgcaaaccggacctctcggccgagacgccagtgtttccggggggcgccgacgagcagacgccgaccgagagcccccggaagtacgtcacgggccacttccgctggggccgcttcggccgcggtaacagcagcggcgcgagccagaagcgtgaggaggaggcggcggcggccgaccccggcttccacggcgatggcgtcgagccgggcctgcgcgaggacaagcgctcctactccatggagcacttccgctggggcaagccggtgggcaagaagcggcgcccggtgaaggtgtacgcgaacggcgcggaggaggagtcggccgaggcctttccgcttgagttcaagcgggagctgaccggggagcggcccgcggcggcgcccggccccgacggcctggggttcggcctggtggctgaggccgaggccgaggcggcagcggccgagaagaaggacgcggccgagaagaaggacgacgggtcctatcgcatggagcacttccgctggggcaccccgcgcaagggcaagcgctacggcggcttcatgacctcggagaagagccagacgccgctggtgacgctgttcaagaacgccatcgtcaagaacgcccacaagaagggccagtga

SEQ ID NO: 16 - protein, 256 aa, Cavia porcellus
MPRSCYSRSGTLLLALLLQISMEVRGWCLESSQCQDLTTERHLLECLRACKPDLSAETPVFPGGADEQTPTESPRKYVTGHFRWGRFGRGNSSGASQKREEEAAAADPGFHGDGVEPGLREDKRSYSMEHFRWGKPVGKKRRPVKVYANGAEEESAEAFPLEFKRELTGERPAAAPGPDGLGFGLVAEAEAEAAAAEKKDAAEKKDDGSYRMEHFRWGTPRKGKRYGGFMTSEKSQTPLVTLFKNAIVKNAHKKGQ

SEQ ID NO: 17 - DNA, 714 bp, Rattus norvegicus
atgccgagattctgctacagtcgctcaggggccctgctgctggccctcctgcttcagacctccatagacgtgtggagctggtgcctggagagcagccagtgccaggacctcaccacggaaagcaacctgctggcttgcatccgggcctgcagactcgacctctcggcggagacgcccgtgtttccaggcaacggagatgaacagcccttgactgaaaatccccggaagtacgtcatgggtcacttccgctgggaccgcttcggcccgagaaacagcagcagtgctggcggctcagcgcagaggcgtgcggaggaagagacggcggggggagatggccgtccggagccaagtccacgggagggcaagcgctcctactccatggagcacttccgctggggcaagccggtgggcaagaagcggcgccctgtgaaggtgtaccccaatgtcgccgagaacgagtcggccgaggcctttcccctagagttcaagagggagctggaaggcgagcagcctgatggcttggagcacgtcctggagccggataccgagaaggccgacgggccctatcgggtggagcacttccgctggggcaacccgcccaaggacaagcgctacggcggcttcatgacctccgagaagagccagacgcccctggtgacgctcttcaagaacgccatcatcaagaacgcgcacaagaagggccagtgagggtgc

SEQ ID NO: 18 - protein, 235 aa, Rattus norvegicus
MPRFCYSRSGALLLALLLQTSIDVWSWCLESSQCQDLTTESNLLACIRACRLDLSAETPVFPGNGDEQPLTENPRKYVMGHFRWDRFGPRNSSSAGGSAQRRAEEETAGGDGRPEPSPREGKRSYSMEHFRWGKPVGKKRRPVKVYPNVAENESAEAFPLEFKRELEGEQPDGLEQVLEPDTEKADGPYRVEHFRWGNPPKDKRYGGFMTSEKSQTPLVTLFKNAIIKNVHKKGQ

SEQ ID NO: 19 - DNA, 708 bp, Mus musculus
atgccgagattctgctacagtcgctcaggggccctgttgctggccctcctgcttcagacctccatagatgtgtggagctggtgcctggagagcagccagtgccaggacctcaccacggagagcaacctgctggcttgcatccgggcttgcaaactcgacctctcgctggagacgcccgtgtttcctggcaacggagatgaacagcccctgactgaaaacccccggaagtacgtcatgggtcacttccgctgggaccgcttcggccccaggaacagcagcagtgctggcagcgcggcgcagaggcgtgcggaggaagaggcggtgtggggagatggcagtccagagccgagtccacgcgagggcaagcgctcctactccatggagcacttccgctggggcaagccggtgggcaagaaacggcgcccggtgaaggtgtaccccaacgttgctgagaacgagtcggcggaggcctttcccctagagttcaagagggagctggaaggcgagcggccattaggcttggagcaggtcctggagtccgacgcggagaaggacgacgggccctaccgggtggagcacttccgctggagcaacccgcccaaggacaagcgttacggtggcttcatgacctccgagaagagccagacgcccctggtgacgctcttcaagaacgccatcatcaagaacgcgcacaagaagggccagtga

SEQ ID NO: 20 - protein, 235 aa, Mus musculus
MPRFCYSRSGALLLALLLQTSIDVWSWCLESSQCQDLTTESNLLACIRACKLDLSLETPVFPGNGDEQPLTENPRKYVMGHFRWDRFGPRNSSSAGSAAQRRAEEEAVWGDGSPEPSPREGKRSYSMEHFRWGKPVGKKRRPVKVYPNVAENESAEAFPLEFKRELEGERPLGLEQVLESDAEKDDGPYRVEHFRWSNPPKDKRYGGFMTSEKSQTPLVTLFKNAIIKNAHKKGQ

SEQ ID NO: 21 - DNA, 759 bp, Gallus gallus
atgcggggcgcgctgtgccacagcctgcccgtggtgctggggctgctgctgtgtcaccccaccaccgccagcggcccatgctgggagaacagcaagtgccaggacctggccaccgaggctggtgttttgcaggcgtgtgccaaggcatgccgtgctgagctgtcggccgaggcacccgtgtacccgggcaatgggcacctgcagcccctctcggagagcatccgcaagtacgtgatgagccatttccgctggaacaagttcggccgtcgcaacagcagcagcggagggcacaaaagggaggaggtggccggcctcgccctgcctgccgcgtcaccccaccaccccgccggggaggaggaagatggagaagggttggaacgagaggaagggaagcgctcctactccatggagcatttccgctggggcaagccggtggggcggaagaggagacccatcaaggtgtaccccaacggggtggacgaggagtcggctgagagttaccccatggagttccggagggagatggcgcccgatggggaccccttcggcctctccgaggaggaggaagaagaggaggaagaggaaggcgaggaggaaaagaaggatggaggctcgtaccgcatgcggcacttccgctggcacgcgccgctgaaggacaagcgctacggcggcttcatgagcttggagcacagccagaccccgctgatgactctgttcaaaaacgccatcgtcaaaagcgcctacaagaagggtcagtga

SEQ ID NO: 22 - protein, 251 aa, Gallus gallus
MRGALCHSLPVVLGLLLCHPTTASGPCWENSKCQDLATEAGVLACAKACRAELSAEAPVYPGNGHLQPLSESIRKYVMSHFRWNKFGRRNSSSGGHKREEVAGLALPAASPHHPAGEEEDGEGLEREEGKRSYSMEHFRWGKPVGRKRRPIKVYPNGVDEESAESYPMEFRREMAPDGDPFGLSEEEEEEEEEEGEEEKKDGGSYRMRHFRWHAPLKDKRYGGFMSLEHSQTPLMTLFKNAIVKSAYKKGQ

SEQ ID NO: 23 - DNA, 780 bp, Bufo marinus
atgttgcagccagggtggagatgtatcctgacaatactcggggcgtttatatttcatgtcggtgaggtcaagagtcagtgctgggagagcggtaaatgtgcagatctgacgagcgaggatgggatactggaatgtattaaagactgcaagatggtcctgtctgcagagtcaccagtgtttcctgggaatggacacatgcaacccctctctgaaaacatcaggaagtatgtcatgagccacttccgctggaataagtttggccgaaggaatagcaccggtggcgatagcaacaacgcaggttacaaacgggaagatatagccaactaccccatatttaacctgttccccactaatgacaaccaaaacacacaagatggcaacatggaagaagaactacgcaggcaagacaacaagaggtcatattctatggaacacttccgatggggtaaaccagtcgggaaaaaaaggagacctattaaggttttcccaagcgatgctgaagaagaatcatctgaaatctscccaacagagtacagaagagagttgtctgtagagtttgactaccccgataccaactctgaagaagacatggacgacagcatgttgatggaaagcccaaatagaaaagatcggaagtataaaatgcatcattttcgatgggaaggtccacccaaagacaaaagatatggaggattcatgacccctgagcgcagtcagactccactaatgactcttttcaaaaatgccattatcaaaaatgcccacaagaagggtcaataa

SEQ ID NO: 24 - protein, 259 aa, Bufo marinus
  misc_feature (174)..(174): Xaa can be any naturally occurring amino acid
MLQPGWRCILTILGAFIFHVGEVKSQCWESGKCADLTSEDGILECIKDCKMVLSAESPVFPGNGHMQPLSENIRKYVMSHFRWNKFGRRNSTGGDSNNAGYKREDIANYPIFNLFPTNDNQNTQDGNMEEELRRQDNKRSYSMEHFRWGKPVGKKRRPIKVFPSDAEEESSEIXPTEYRRELSVEFDYPDTNSEEDMDDSMLMESPNRKDRKYKMHHFRWEGPPKDKRYGGFMTPERSQTPLMTLFKNAIIKNAHKKGQ

SEQ ID NO: 25 - DNA, 669 bp, Cyprinus carpio
atggtgaggggagagaggatgttgtgtcctgcttggctcttggctctggctgttctgtgtgcggctggatctgaagtcagagctcagtgtatggaggacgcccgctgcagagacctcaccactgatgagaacatcttggactgcatacagctatgcaggtctgatctgacagatgaaacccccgtctaccctggagaaagccatttgcagcctccctctgagctggagcaaaccgaggtcctcgtacccctgtccccagcggccctcgctcctgctgagcaaatggaccccgagtccagccctcagcacgagcacaagcgctcctactccatggagcatttccgctggggaaagccagtgggtcgcaagcgcaggcctatcaaggtgtacaccaacggcgtggaggaggaatccaccgagactctcccagctgagatgaggcgcgagctggctacaaacgagatcgactatcctcaagaggagggcgctttaaaccagcaggataagaaggatggctcctacaaaatgagccatttccgctggagcagcccgcctgctagcaagcgctatggaggcttcatgaagtcctgggacgagcgcagtcagaaaccccttctcacgctcttcaaaaacgtcataaacaaagagcaccagaagaaggaccagtga

SEQ ID NO: 26 - protein, 222 aa, Cyprinus carpio
MVRGERMLCPAWLLALAVLCAAGSEVRAQCMEDARCRDLTTDENILDCIQLCRSDLTDETPVYPGESHLQPPSELEQTEVLVPLSPAALAPAEQMDPESSPQHEHKRSYSMEHFRWGKPVGRKRRPIKVYTNGVEEESTETLPAEMRRELATNEIDYPQEEGALNQQDKKDGSYKMSHFRWSSPPASKRYGGFMKSWDERSQKPLLTLFKNVINKEHQKKDQ

SEQ ID NO: 27 - DNA, 669 bp, Danio rerio
atggtgaggggagtgaggatgttgtgtcctgcttggctcttggctctggctgttctctgcgcaggaggatctgaagtcagagctcagtgttgggaaaatgcccgctgtcgagacctcagcacagaggagaacatcttggaatgcatacaattatgcaggtctgaacttacagatgaaacccccgtctaccctggagaaagccatctacagcctccctccgagccggagcaaatcgacctcctcgcacacctttcccctgtagcactcgcagcccctgaacagatagagccggagtccggccctcgacacgaccacaagcgctcctactccatggaacacttccggtggggcaaaccggtcggccgcaaacgcagacccatcaaggtgtacacgaacggcgtggaagaggaatccgccgaaacgcttccggaagagatgagacgcgagctggcaaataacgaggtcgactatccgcaagaagagatgcctttaaacccactgggaaagaaggaccccccctacaaaatgacccatttccgctggagcgtcccgccggctagcaagcgctatggaggcttcatgaagtcctgggacgagcgtgctcagaaaccactgctcacactcttcaaaaacgtaatgcataaaggccaaccgaggaaggatgagtga

SEQ ID NO: 28 - protein, 222 aa, Danio rerio
MVRGVRMLCPAWLLALAVLCAGGSEVRAQCWENARCRDLSTEENILECIQLCRSELTDETPVYPGESHLQPPSEPEQIDLLAHLSPVALAAPEQIEPESGPRHDHKRSYSMEHFRWGKPVGRKRRPIKVYTNGVEEESAETLPEEMRRELANNEVDYPQEEMPLNPLGKKDPPYKMTHFRWSVPPASKRYGGFMKSWDERAQKPLLTLFKNVMHKGQPRKDE

SEQ ID NO: 29 - DNA, 792 bp, Rana catesbeiana
atgttgcagccagtctggcacgcctgtatcctggcaatacttggggtgttcatatttcatgtcggagaggtccggagccagtgctgggaaagcaataagtgtacagatttaagcagcgaagatggcattctggaatgtatcaaagcatgcaagatggacctctctgcagaatctcccgtgtttcccggcaatggccacatccagcccctttctgaaaacatcaggaaatatgtcatgagccactttcgctggaataaatttggtagaaggaacagcaccagcaatgacaacaacaacaacaatggtggctataagcgggaggatattgccaactaccctatattgaacctgttccttggcagcgacaaccaaaacacacaggagggaattatggaagatgacgccttggataggcaagacagcaaaaggtcttattccatggagcacttccgatggggaaaacccgtcggcaagaagaggaggcctatcaaagttttccccacagatgctgaagaagagtcctcagaaagtttccccattgagctgagaagagagctctctctagagtttgactatcctgacaccaactccgaagaagaattggataatggcgagctgctagaaggtccagttaaaaaaggtaggaagtacaaaatgcaccatttccgatgggaaggacctcccaaagacaagcggtatggtggatttatgaccccagagagaagccagacacctttaatgactcttttcaagaatgctataattaagaacgcccacaaaaagggccagtag

SEQ ID NO: 30 - protein, 263 aa, Rana catesbeiana
MLQPVWHACILAILGVFIFHVGEVRSQCWESNKCTDLSSEDGILECIKACKMDLSAESPVFPGNGHIQPLSENIRKYVMSHFRWNKFGRRNSTSNDNNNNNGGYKREDIANYPILNLFLGSDNQNTQEGIMEDDALDRQDSKRSYSMEHFRWGKPVGKKRRPIKVFPTDAEEESSESFPIELRRELSLEFDYPDTNSEEELDNGELLEGPVKKGRKYKMHHFRWEGPPKDKRYGGFMTPERSQTPLMTLFKNAIIKNAHKKGQ

SEQ ID NO: 31 - protein, 272 aa, Monodelphis domestica
MPKPSWSYLGALLVAVLFQASVEVHGWCLQASNCRDSKAEDGLVECIKSCKMDLSAESPVFPGNGQYEPLSENIRKYVMSHFRWNKFGRRNISSGSISSDGGNVGQKRQELMQGDFLDLPPPGVWGEDEEMQEGLPLIRKARELQNKRSYSMEHFRWGKPVGKKRRPVKIYPNGVEEESAESYPVEIRRDLPMKINFPEYPELAIDEEEAAKEVYEEKVKKDGGGYKMEHFRWGTPPKDKRYGGFMISEKSHTPLMTLFKNAIIKNGHKKGQ

SEQ ID NO: 32 - protein, 263 aa, Ovis aries
  misc_feature (184)..(184): Xaa can be any naturally occurring amino acid
MPRLCSSRSGALLLVLLLQASMEVRGWCLESSQCQDLTTESNLLACIRACKPDLSAETPVFPGNGDEQPLTENPRKYVMGHFRWDRFGRRNGSSSFGAGGAAQKREEEVAVGEGPGPRGDGAETGPREDKRSYSMEHFRWGKPVGKKRRPVKVYPNGAEDESAQAFPLEFKRELTGERLEQARXPEAQAESAAARAELEYGLVAEAEAAEKKDSGPYKMEHFRWGSPPKDKRYGGFMTSEKSQTPLVTLFKNAIIKNAHKKGQ

SEQ ID NO: 33 - protein, 212 aa, Ovis aries
  misc_feature (120)..(121): Xaa can be any naturally occurring amino acid
PDLSAETPVFPGNCDEQPLTENPRKYVMGHFRWDRFGRRNGSSSFGAGGAAQKREEEVAVGEGPGPRGDGAETGPREDKRSYSMEHFRWGKPVGKKRRPVKVYPNGAEDESAQAFPLEFXXELTGERLEQARGPEAQAESAAARAELEYGLVAEAEAAEKKDSGPYKMEHFRWGSPPKDKRYGGFMTSEKSQTPLVTLFKNAIIKNAHKKGQ

SEQ ID NO: 34 - protein, 263 aa, Rana catesbeiana; identical to SEQ ID NO: 30

SEQ ID NO: 35 - protein, 258 aa, Spea multiplicata
MLCPVWSCLFAVLGVFVFHVGEVRGQCWQSAKCMDLESEDGILECIKACKTDLSAESPIFPGNGHLQPLAENVRKYVMSHFRWNKFGRRNTTGNEGNSGSKREDIANYPIFNLFPSSNGQNTEDNMWKKYQDRQDNKRSYSMEHFRWGKPVGRKRRPIKVFPNGMEEESSESYPMELRRELSLEDDYPEIDSEDDLDYNDLLSMPKFKGGDYRIHHFRWGSPPKDKRYGGFMTPERSQTPLMTLFKNAIIKNAHKKAQ

SEQ ID NO: 36 - protein, 259 aa, Xenopus laevis
MFRPLWGCFLAILGICIFHIGEVQSQCWESSRCADLSSEDGVLECIKACKTDLSAESPVFPGNGHLQPLSESIRKYVMTHFRWNKFGRRNSTGNDGSNTGYKREDISSYPVFSLFPLSDQNAPGDNMEEEPLDRQENKRAYSMEHFRWGKPVGRKRRPIKVYPNGVEEESAESYPMELRRELSLELDYPEIDLDEDIEDNEVKSALTKKNGNYRMHHFRWGSPPKDKRYGGFMTPERSQTPLMTLFKNAIIKNSHKKGQ

SEQ ID NO: 37 - protein, 262 aa, Necturus maculosus
  misc_feature (129)..(129): Xaa can be any naturally occurring amino acid
MLKPVWSCLFATLGALLCQTVVAHSQCWESSKCRDLATEGSVLECIKACKVELSAESPVYPGNGHMQPLSENIRKYVMSHFRWNQFGRKNSTVASGNGAGSKREELSGNPIISLFTTSESQSSGAHDSXKEGEVMDRQDNKRSYSMEHFRWGKPVGRKRRPIKVYPNGVEEESSESYPLELKRDLSLGLEYPEFDSQEGLENNEVMVVLPEKKDGNYRMHHFRWGSPPKDKRYGGFMTPERSQTPLMTLFKNAIKNAHKKGQ

SEQ ID NO: 38 - protein, 262 aa, Amphiuma means
MLRPVWSCLPATLGALLCQTAGANSQCWESSKCRDLATEGSVLECIKACKVELSAESPVYPGNGHMQPLSENIRKYVMSHFRWNKFGRKNSTSVSGNSAGNKREELSNNPIISLFTTSESQSSGADDGNKEGEAMERQDSKRSYSMEHFRWGKPVGRKRRPIKVYPNGVEEESSESYPLELRRDLSLGLDYPDSDSQEGLENNEITTGLTKKNDKQYRIGHFRWGSPLKDKRYGGFMTPERSQTPLMTLFKNAIKNAHKKGQ

SEQ ID NO: 39 - protein, 261 aa, Pelodiscus sinensis
MLKPVRSGLLAILGVLLFHADGGVHSQCWDSSRCRELSTDAGLLECIKACKMDLSDESPMYPGNGHLQPLSENIRKYVMSHFRWNKFGRKNSSSSVAGHKREEIPSHLLLGLFPDVAPAQRGDDGEGGAALERQDSKRSYSMEHFRWGKPVGRKRRPIKVYPSEVEEESAESYPPEFRRDLSMELDYPEFESLEDPESEEALVSEEAEKKDGNSYKMHHFRWNAPPKDKRYGGFMTSESSQTPLMTLFKNAIIKNAYKKGQ

SEQ ID NO: 40 - protein, 187 aa, Pan troglodytes
SAETPMFPGNGDEQPLTENPRKYVMGHFRWDRFGRRNSSSSSSGSGAGQKREDVSAGEDRGPLPEGGPEPRSDGAKPGPREGKRSYSMEHFRWGKPVGKKRRPVKVYPNGAEDESAEAFPLEFKRELTGQRPREGDGPDGPADDGAGAQADLEHSLLVAAEKKDEGPYRMEHFRWGSPPKDKRYGGF

SEQ ID NO: 41 - DNA, 20 bp, Artificial
  SYNTHETIC OLIGONUCLEOTIDE
gcttgcaaactcgacctctc

SEQ ID NO: 42 - DNA, 20 bp, Artificial
  SYNTHETIC OLIGONUCLEOTIDE
cttgatgatggcgttcttga

SEQ ID NO: 43 - DNA, 22 bp, Artificial
  SYNTHETIC OLIGONUCLEOTIDE
agggcatcagaaggcctgacca

SEQ ID NO: 44 - DNA, 22 bp, Artificial
  SYNTHETIC OLIGONUCLEOTIDE
cttgaagaagcggcagtagcac

SEQ ID NO: 45 - DNA, 20 bp, Artificial
  SYNTHETIC OLIGONUCLEOTIDE
gcttgcaaactcgacctctc

SEQ ID NO: 46 - DNA, 20 bp, Artificial
  SYNTHETIC OLIGONUCLEOTIDE
cttgatgatggcgttcttga

SEQ ID NO: 47 - DNA, 21 bp, Artificial
  SYNTHETIC OLIGONUCLEOTIDE
atggggctgtgtggactgacc

SEQ ID NO: 48 - DNA, 22 bp, Artificial
  SYNTHETIC OLIGONUCLEOTIDE
gtcaggagagcaagtttcattt

SEQ ID NO: 49 - DNA, 22 bp, Artificial
  SYNTHETIC OLIGONUCLEOTIDE
agggcatcagaaggcctgacca

SEQ ID NO: 50 - DNA, 22 bp, Artificial
  SYNTHETIC OLIGONUCLEOTIDE
cttgaagaagcggcagtagcac

SEQ ID NO: 51 - DNA, 18 bp, Artificial
  SYNTHETIC OLIGONUCLEOTIDE
agcaaccggagtggcagt

SEQ ID NO: 52 - DNA, 18 bp, Artificial
  SYNTHETIC OLIGONUCLEOTIDE
ggccacgatcaaggagag

SEQ ID NO: 53 - DNA, 19 bp, Artificial
  SYNTHETIC OLIGONUCLEOTIDE
agtctctggggaaggggca

SEQ ID NO: 54 - DNA, 20 bp, Artificial
  SYNTHETIC OLIGONUCLEOTIDE
caactgatgatgatcccgac

(19) United States
(12) Patent Application Publication
(10) Pub. No.: US 2005/0002905 A1
(43) Pub. Date: Jan. 6, 2005

(54) RAAV vector-based pro-opiomelanocortin compositions and methods of use

(75) Inventors: Philip J. Scarpace, Gainesville, FL (US); Gang Li, Gainesville, FL (US)

Correspondence Address: WILLIAMS, MORGAN & AMERSON, P.C., 10333 RICHMOND, SUITE 1100, HOUSTON, TX, 77042, US

(73) Assignee: The University of Florida

(21) Appl. No.: 10/822,613

(22) Filed: Apr. 12, 2004

Related U.S. Application Data
(60) Provisional application No. 60/462,496, filed on Apr. 11, 2003.

(51) Int. Cl. A61K 48/00; C12N 15/861

(52) U.S. Cl. 424/93.2; 435/456

(57) ABSTRACT

Disclosed are methods for the use of pro-opiomelanocortin-encoding polynucleotides in the creation of transformed host cells and transgenic animals. In particular, the use of recombinant adeno-associated viral (rAAV) vector compositions, virions, and pluralities of virus particles that comprise a nucleic acid segment that expresses one or more mammalian pro-opiomelanocortin polypeptides in suitably transformed host cells is described. Also disclosed are methods for the treatment and amelioration of symptoms of a variety of conditions and disorders in an animal, including hyperinsulinemia, obesity, adiposity, overeating, and related eating disorders.

The present application claims priority to U.S. Provisional Patent Application Ser. No. 60/462,496 filed Apr. 11, 2003, the entire contents of which is specifically incorporated herein by reference in its entirety. The United States government has certain rights in the present invention pursuant to a grant from the Medical Research Service of the Department of Veterans Affairs and Grant Numbers AG-17047 and AG-20985 from the National Institutes of Health.

### 1.0 BACKGROUND OF THE INVENTION

1.1 Field of the Invention

The present invention relates generally to the fields of molecular biology and virology, and in particular, to methods for using recombinant adeno-associated virus (rAAV) compositions that express nucleic acid segments encoding pro-opiomelanocortin polypeptides useful in the treatment of certain human disorders. In illustrative embodiments, the invention concerns the use of rAAV-pro-opiomelanocortin compositions in a variety of investigative, diagnostic and therapeutic regimens, including the prevention and treatment of diseases and disorders such as obesity, weight gain, and hyperinsulinemia. Methods and compositions are provided for preparing rAAV vector-based pro-opiomelanocortin constructs for use in the preparation of medicaments useful in central gene therapy of various eating disorders, reducing food intake, controlling adiposity, and managing body weight gain.

1.2 Description of the Related Art

An estimated 54 percent of adults in the United States are overweight, and greater than 27% are considered clinically obese, according to reports from the National Institutes of Health. Treating obesity and its related conditions, including high blood pressure, heart disease and diabetes, is estimated to cost more than $45 billion annually, according to an August, 1996 Scientific American article. Likewise, some 20-40% of pets, such as cats and dogs, are also considered to be obese by current veterinary medical standards. With the incidence of obesity on the rise in the United States in both the human and pet populations, there is a need to develop therapies that will alleviate the symptoms associated with obesity in both man and his animal companions.

Melanocortins (MCs) are peptides cleaved from a common precursor, pro-opiomelanocortin (POMC). The central MC system plays a critical role in the homeostatic regulation of body weight. Reduced expression of hypothalamic POMC is associated with obesity syndromes due to mutations in the leptin receptor or other genes (tubby, Nhlh2, etc.), due to hypothalamic damage, and most commonly, aging. Moreover, mutations in the POMC gene cause obesity in humans. POMC has been known by various other names in the literature, including, for example, adrenocorticotropin, β-lipotropin, α-melanocyte stimulating hormone, corticotropin-lipotropin precursor, β-melanocyte stimulating hormone, and β-endorphin, among others.

The central melanocortin system plays a critical role in the homeostatic regulation of body weight (Cone, 1999; Fan et al., 1997; Huszar et al., 1997; Mizuno and Mobbs, 1999; Butler et al., 2000). Melanocortins (MCs) are bioactive peptides derived from a common pre-hormone, pro-opiomelanocortin (POMC), one of which, α-melanocyte stimulating hormone (α-MSH), is a major regulator of feeding and body weight homeostasis. Reduced expression of hypothalamic POMC is associated with obesity syndromes caused by mutations in the leptin receptor (Mizuno et al., 1998; Kim et al., 2000), or other genes (tubby, Nhlh2, etc.) (Guan et al., 1998; Good et al., 1997); by hypothalamic damage (Bergen et al., 1998); and perhaps most common, by aging (Mobbs et al., 2001). That reduced hypothalamic POMC mRNA could contribute to the obese phenotypes in these models is suggested by the observation that mutations in the POMC gene cause obesity in mice (Yaswen et al., 1999) and humans (Krude et al., 1998). However, it is still unclear whether normalization of central POMC tone can reverse obese phenotypes.

The MC system is presumably located downstream of hypothalamic leptin receptor-mediated signal transduction. Previous studies suggest acute activation of central MC system by MC agonists reduces food intake and fat mass in leptin-resistant animal models. However, these responses were only transient.

Leptin, an adipocyte-derived hormone, acts on satiety and appetite centers in the hypothalamus to both reduce food consumption and increase energy expenditure (Friedman and Halaas, 1998; Schwartz et al., 1996; Scarpace et al., 1997). Recent evidence suggests that the melanocortin system may be located downstream of the hypothalamic leptin-signaling pathway. Leptin activates POMC- and agouti-related protein (AgRP)-containing neurons of the ventrolateral and ventromedial arcuate nucleus, respectively, resulting in an increase in the expression of POMC and a reduction in AgRP (Schwartz et al., 1996; Schwartz et al., 1997; Cheung et al., 1997; Baskin et al., 1999; Elias et al., 2000). In addition, leptin-induced suppression of food intake is effectively blocked by an MC ¾ receptor antagonist (Seeley et al., 1997), and the leptin-mediated induction of uncoupling protein 1 (UCP1), an important thermogenic protein in the brown adipose tissue (BAT), is also attenuated by central MC receptor antagonism (Satoh et al., 1998).

Genetically obese falfa Zucker rats with a recessive mutation of the leptin receptor gene develop severe, early-onset obesity associated with hyperphagia, hyperleptinemia and hyperinsulinemia (Bray, 1977; lida et al., 1996).

### 1.3 DEFICIENCIES IN THE PRIOR ART

Currently, there are limited pharmacological approaches to preventing or treating complex eating disorders, obesity, hyperinsulinemia, and related dysfunction in affected mammals. Many such methods introduce undesirable side-effects, and do not overcome the problems associated with traditional modalities and treatment regimens for such conditions. Thus, the need exists for an effective treatment that circumvents the adverse effects and provides more desirable results, with longer acting effects, and improved compliance in both human and veterinary patients. There also is needed an effective treatment for curbing the body weight gain, improving insulin sensitivity, and reducing cholesterol levels in mammals, particularly in obese humans as well as veterinary animals, such as domesticated cats and dogs, or other pets. In addition, methods for delivery of polynucleotides to a host cell that express a pro-opiomelanocortin polypeptide useful in the amelioration of such eating disorders and related conditions, and in particular, administration of specific rAAV-based pro-opiomelanocortin polynucleotide constructs to a mammal are particularly desirable.

### 2.0 SUMMARY OF THE INVENTION

The present invention overcomes these and other limitations inherent in the prior art by providing new rAAV-based genetic constructs that encode one or more mammalian pro-opiomelanocortin polypeptides for the treatment or amelioration of various disorders, including those that result from a deficiency in, or an absence of, sufficient pro-opiomelanocortin polypeptides in the cells of such mammals. In particular, the invention provides genetic constructs encoding one or more mammalian pro-opiomelanocortin polypeptides, for use in the treatment of such conditions as obesity, hyperinsulinemia, anorexia, weight gain, and a variety of eating disorders. Likewise, the invention provides genetic constructs that encode one or more pro-opiomelanocortin polypeptides useful in the prevention, treatment or amelioration of symptoms of various human disorders that manifest or are exacerbated by a deficiency or absence of physiologically-normal levels of pro-opiomelanocortin polypeptides in selected cells of such human beings. Also provided is a means for decreasing risk factors for atherosclerosis, hypertension, diabetes and other obesity-related disorders in animals treated with the therapeutic medicaments of the present invention.

The invention provides compositions and methods for treating or ameliorating such a pro-opiomelanocortin polypeptide deficiency in a mammal, and particularly for treating or reducing the severity or extent of deficiency in a human manifesting one or more of the disorders linked to a deficiency in such polypeptides. In a general sense, the method involves administration of one or more rAAV-based genetic construct(s) that comprise a polynucleotide sequence comprising at least one gene encoding a pro-opiomelanocortin polypeptide to a selected mammalian recipient, in an amount and for a period of time sufficient to treat or ameliorate the deficiency in the animal suspected of suffering from such a disorder. The invention also provides an rAAV virion, and pluralities of such viral particles that comprise one of more of such rAAV vectors. In certain therapeutic embodiments, and in methods involving the compositions for use in the preparation of a medicament for treating or preventing such disorders, the vectors, virions, or viral particles of the present invention are preferably formulated in one or more pharmaceutically-acceptable vehicles, diluents, buffers, or such like that are biocompatible or biologically-acceptable when administered to a mammal.

The present invention provides an adeno-associated viral vector comprising a polynucleotide that comprises a nucleic acid segment encoding a pro-opiomelanocortin polypeptide (including for example, but not limited to, mammalian POMC polypeptides including those of human, primate, murine, porcine, bovine, ovine, feline, canine, equine, epine, caprine, or lupine origin) operably linked to a promoter capable of expressing the segment in a host cell comprising the vector. In exemplary embodiments, the expressed polypeptide activates the central melanocortin pathway in cells of a mammal that expresses the vector.

In illustrative embodiments, the invention provides a vector wherein the nucleic acid segment encodes a polypeptide that is at least 85% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

Preferably, the invention provides an rAAV vector, and compositions comprising it, wherein the nucleic acid segment encodes a polypeptide that is at least 90% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

More preferably, the invention provides an rAAV vector, and compositions comprising it, wherein the nucleic acid segment encodes a polypeptide that is at least 95% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

More preferably still, the invention provides an rAAV vector, and compositions comprising it, wherein the nucleic acid segment encodes a polypeptide that is at least 98% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

In exemplary embodiments, the invention provides an rAAV vector, and compositions comprising it, in which the nucleic acid segment encodes a polypeptide that comprises, consists essentially of, or consists of the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

Preferably, the invention provides an rAAV vector (as well as virions, virus particles, and compositions comprising it), wherein the nucleic acid segment comprises, consists essentially of, or consists of, a nucleotide sequence that is at least 80% homologous to the nucleotide sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29.

More preferably, the invention provides an rAAV vector (as well as virions, virus particles, and compositions comprising it) wherein the nucleic acid segment comprises, consists essentially of, or consists of, a nucleotide sequence that is at least 85% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ BD NO:21, SEQ ID NO:23, SEQ ID NO:25, SEQ ID NO:27, or SEQ ID NO:29.

Still more preferably, the invention provides an rAAV vector (as well as virions, virus particles, and compositions comprising it), wherein the nucleic acid segment comprises, consists essentially of, or consists of, a nucleotide sequence that is at least 90% homologous to the sequence of any one of SEQ ID NO:1, SEQ ID NO:3, SEQ ID NO:5, SEQ ID NO:7, SEQ BD NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29.

Even more preferably, the invention provides an rAAV vector (as well as virions, virus particles, and compositions comprising it), wherein the nucleic acid segment comprises, consists essentially of, or consists of, a nucleotide sequence that is at least 95% homologous to the sequence of any one of SEQ ID NO:1, SEQ ID NO:3, SEQ ID NO:5, SEQ ID NO:7, SEQ ID NO:9, SEQ ID NO:11, SEQ BD NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29.

In exemplary embodiments, the invention provides an rAAV vector (as well as virions, virus particles, and compositions comprising it), wherein the nucleic acid segment comprises, consists essentially of, or consists of, the nucleotide sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29.

In exemplary embodiments, the vectors of the present invention may comprise one or more constitutive, inducible, or tissue-specific promoters, including for example, but not limited to, one or more promoters selected from the group consisting of a CMV promoter, a β-actin promoter, a hybrid CMV enhancer/β-actin promoter, an EF1 promoter, an U1a promoter and an U1b promoter. Exemplary inducible promoters include, but are not limited to, a promoter selected from the group consisting of a Tet-inducible promoter and a VP16-LexA promoter.

In certain embodiments, the vectors of the present invention may also optionally comprise one or more enhancers, enhancer elements, or regulatory sequences operably linked to the nucleic acid segment the comprises the therapeutic gene. Examples of such enhancer elements include, but are not limited to, the CMV enhancer.

Likewise, in some embodiments, the vectors of the present invention may also optionally comprise one or more post-transcriptional regulatory elements, such as for example, the woodchuck hepatitis virus post-transcription regulatory element.

The invention also provides an adeno-associated viral vector comprising a polynucleotide that comprises a nucleic acid segment that is at least 90% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein such a nucleic acid segment encodes a biologically-active pro-opiomelanocortin polypeptide operably linked to a promoter capable of expressing such a segment in a host cell that comprises the vector.

Likewise, the invention also provides an adeno-associated viral vector comprising a polynucleotide that comprises a nucleic acid segment that is at least 92% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein such a nucleic acid segment encodes a biologically-active pro-opiomelanocortin polypeptide operably linked to a promoter capable of expressing such a segment in a host cell that comprises the vector.

More preferably, the invention discloses and claims adeno-associated viral vectors that comprise at least a first polynucleotide that comprises a nucleic acid segment that is at least 94% homologous to the sequence of any one of SEQ ID NO: 1, SEQ ID NO:3, SEQ ID NO:5, SEQ ID NO:7, SEQ ID NO: 9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein such a nucleic acid segment encodes a biologically-active pro-opiomelanocortin polypeptide operably linked to a promoter capable of expressing such a segment in a host cell that comprises the vector.

More preferably still, the invention discloses and claims adeno-associated viral vectors that comprise at least a first polynucleotide that comprises a nucleic acid segment that is at least 96% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein such a nucleic acid segment encodes a biologically-active pro-opiomelanocortin polypeptide operably linked to a promoter capable of expressing such a segment in a host cell that comprises the vector.

Even more preferably, the invention provides adeno-associated viral vectors that comprise at least a first polynucleotide that comprises a nucleic acid segment that is at least 98% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein such a nucleic acid segment encodes a biologically-active pro-opiomelanocortin polypeptide operably linked to a promoter capable of expressing such a segment in a host cell that comprises the vector.

In certain illustrative embodiments, the invention provides adeno-associated viral vectors that comprise at least a first polynucleotide that comprises a nucleic acid segment that comprises, consists essentially of, or consists of a sequence that is at least 99% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein such a nucleic acid segment encodes a biologically-active pro-opiomelanocortin polypeptide operably linked to a promoter capable of expressing such a segment in a host cell that comprises the vector.

In additional embodiments, the invention also provides an adeno-associated viral vector comprising at least a first polynucleotide that comprises a nucleic acid segment that encodes a mammalian pro-opiomelanocortin polypeptide that is at least 91% identical to the amino acid sequence of any one or SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, operably linked to a promoter capable of expressing the nucleic acid segment in a host cell that comprises the vector, wherein such an encoded polypeptide activates the central melanocortin pathway in a mammal that expresses such a vector.

Likewise, the invention provides an adeno-associated viral vector comprising at least a first polynucleotide that comprises a nucleic acid segment that encodes a mammalian pro-opiomelanocortin polypeptide that is at least 93% identical to the amino acid sequence of any one or SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, operably linked to a promoter capable of expressing the nucleic acid segment in a host cell that comprises the vector, wherein such an encoded polypeptide activates the central melanocortin pathway in a mammal that expresses such a vector.

More preferably, the invention also provides an adeno-associated viral vector comprising at least a first polynucleotide that comprises a nucleic acid segment that encodes a mammalian pro-opiomelanocortin polypeptide that is at least 95% identical to the amino acid sequence of any one or SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, operably linked to a promoter capable of expressing the nucleic acid segment in a host cell that comprises the vector, wherein such an encoded polypeptide activates the central melanocortin pathway in a mammal that expresses such a vector.

Even more preferably, the invention provides an adeno-associated viral vector comprising at least a first polynucleotide that comprises a nucleic acid segment that encodes a mammalian pro-opiomelanocortin polypeptide that is at least 97% identical to the amino acid sequence of any one or SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, operably linked to a promoter capable of expressing the nucleic acid segment in a host cell that comprises the vector, wherein such an encoded polypeptide activates the central melanocortin pathway in a mammal that expresses such a vector.

Likewise, the invention also provides an adeno-associated viral vector comprising at least a first polynucleotide that comprises a nucleic acid segment that encodes a mammalian pro-opiomelanocortin polypeptide that is at least 99% identical to the amino acid sequence of any one or SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, operably linked to a promoter capable of expressing the nucleic acid segment in a host cell that comprises the vector, wherein such an encoded polypeptide activates the central melanocortin pathway in a mammal that expresses such a vector.

In certain illustrative embodiments, the invention provides adeno-associated viral vectors that comprise at least a first polynucleotide that comprises a nucleic acid segment that encodes a mammalian pro-opiomelanocortin polypeptide that comprises, consists essentially of, or consists of the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

The invention also provides recombinant adeno-associated virus virions, viral particles, and pluralities or populations of such virions and/or viral particles that comprise, or substantially all of which comprise one or more of the rAAV vectors described herein. Likewise, the invention also discloses and claims compositions that comprise one or more of the polynucleotides, polypeptides, vectors, virus particles, and/or virions described herein. Such compositions may further optionally comprise at least one or more physiologically-acceptable buffers, diluents, or pharmaceutically-acceptable excipients, carriers, or vehicles, as described herein. Example of these include, but are not limited to, compositions that further comprise one or more buffers, liposomes, lipids, lipid complexes, microspheres, microparticles, nanospheres, nanocapsules, or nanoparticles. Such compositions may be formulated for administration to one or more tissues, organs, cells, or systems of a mammal, with formulations for administration to veterinary animals, and primates, including humans, being particularly preferred.

In embodiments where administration of pharmaceutical formulations of the invention to a mammal is contemplated, such compositions may be formulated for systemic, intramuscular, intracerebroventricular, intranasal, intraocular, oral, sublingual, intradermal, transdermal, intramuscular, intraperitoneal, or by direct injection or administration to one or more cells, tissues, or organs of the mammal using one or more of the conventional methodologies for AAV administration, as known and routinely used by one of skill in these arts. In methods employing such compositions, they may be formulated by any conventional method, such that the vectors, virions, or viral particles comprised within such compositions are introduced into suitable cell(s), organ(s), or tissue(s) by suitable means, including for example, by infection, transfection, or by direct injection.

The invention also provides diagnostic and therapeutic kits for diagnosing, preventing, treating, and/or ameliorating the symptoms of one or more diseases, disorders, and/or dysfunctions caused by a lack of functional pro-opiomelanocortin polypeptide activity in a mammal, or by a deficiency in the level of POMC polypeptide, or a deficiency in the activity of native POMC protein in a mammal. Such kits typically will comprise: (i) one or more of the rAAV-POMC vectors, virions viral particles, or compositions as disclosed herein; and (ii) instructions for using the kit.

The invention also provides uses of the disclosed compositions in therapy, and particularly in therapeutic methods for treating obesity, adiposity, polyphagia, hyperinsulinemia, or controlling food intake, or managing the body weight gain in a mammal. These methods generally comprise at least the step of introducing into a cell or tissue of the mammal, a therapeutically-effective amount of one or more of the disclosed rAAV-POMC vectors (as well as virions, pluralities of virus particles, or compositions comprising them), for a time effective to treat the intended condition, or to ameliorate the symptoms of the disease or disorder. Preferably, the mammal is a human under the care of a physician or suitable health care professional, or in the case of non-human animals, the mammal is preferably under the care of a veterinarian.

Also provided is a method for treating or ameliorating the symptoms of a pro-opiomelanocortin polypeptide deficiency condition in a mammal. The method generally involves at least the step of administering to such a one or more of the rAAV-POMC vectors, virions viral particles, or compositions as disclosed herein, in an amount and for a time sufficient to treat or ameliorate the symptoms of such a deficiency in the mammal.

For example, the method may be used to treat or ameliorate the symptoms of eating disorders, including for example, those selected from the group consisting of obesity, overeating, and bingeing. Alternatively, the method may be used to provide the animal with an amount of the disclosed POMC compositions required over a sufficient course of treatment to decrease the body weight of the mammal, or to decrease the rate of body weight gain in the mammal.

Likewise, the invention provides a method for providing to a mammal in need thereof, a therapeutically-effective amount of a pro-opiomelanocortin peptide, polypeptide, protein, antibody, antisense molecule or ribozyme. Such method generally involves introducing into suitable cell(s), organ(s), and/or tissue(s) of the mammal, a biologically-effective amount of a pro-opiomelanocortin peptide, polypeptide, protein, antibody, antisense molecule or ribozyme; for a time effective to provide the mammal with a therapeutically-effective amount of the pro-opiomelanocortin polypeptide. Such methodologies may be involved directly using an in vivo or in situ treatment regimen, where the rAAV-POMC vectors or POMC compositions are directly introduced into the living organism itself, or alternatively, the process may involve treating one or more cell(s), tissue(s), and/or organ(s) of the animal in an ex vivo, or in vitro, fashion, and then reintroducing such treated cells, tissues, or organ back into the body of the animal in need of the treatment.

### 2.1 RAAV-PRO-OPIOMELANOCORTIN VECTOR COMPOSITIONS

In a first embodiment, the invention provides an rAAV vector comprising a polypeptide that comprises at least a first nucleic acid segment that encodes a mammalian pro-opiomelanocortin peptide or polypeptide, and in particular, a biologically-active pro-opiomelanocortin (POMC) polypeptide, or biologically-active fragment thereof, operably linked to at least a first promoter capable of expressing the nucleic acid segment in a suitable host cell transformed with such a vector. In preferred embodiments, the nucleic acid segment encodes a mammalian, and in particular, a human POMC polypeptide, such as for example, biologically-active contiguous amino acid sequences that comprise, consist essentially of, or consist of, one or more of the polypeptide sequences as disclosed in any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

In addition to therapeutic polynucleotides and polypeptides of human origin, the invention also encompasses treatment modalities involving the use of one or more other mammalian pro-opiomelanocortin-encoding nucleotides or pro-opiomelanocortin peptides, proteins, or polypeptides, as may be desirable in the treatment of humans, or other mammals (such as for example, in veterinary medicine therapies), and as such, the rAAV vectors may comprise pro-opiomelanocortin-encoding sequences that are derived in whole or in part of native, mutated, or synthetically-modified sequences, including those of primate, murine, porcine, feline, canine, bovine, ovine, equine, epine, caprine, or lupine origin. In an example presented herein as an illustrative embodiment of the practice of the invention, the rAAV- pro-opiomelanocortin constructs comprise at least a first genetic sequence that encodes a human pro-opiomelanocortin peptide, polypeptide, or protein, to provide therapeutic levels of the selected protein, e.g., POMC, to the transfected cells.

Alternatively, the therapeutic constructs of the invention may encompass nucleic acid segments that encode modified POMC polypeptides obtained from any mammalian origin, and engineered by the hand of man to produce more desirable properties or characteristics. For example, nucleic acids, peptides, proteins, and polypeptides of murine, primate, ovine, porcine, bovine, equine, epine, caprine, canine, feline, avian, amphibian, and/or lupine origin, may be used in their native or unmodified form, but also may be modified or site-specifically mutagenized, and/or otherwise genetically modified to be expressed in human cells such that their pro-opiomelanocortin biological activity is retained, increased, or prolonged.

Preferred rAAV vector backbones for the practice of the present invention include, but are not limited to, rAAV serotype 1 (rAAV1), rAAV serotype 2 (rAAV2), rAAV serotype 3 (rAAV3), rAAV serotype 4 (rAAV4) and rAAV serotype 5 (rAAV5), or rAAV serotype 6 (rAAV6) vectors, or derivates thereof.

The polynucleotides comprised in the vectors and viral particles of the present invention preferably comprise at least a first constitutive or inducible promoter operably linked to the nucleic acid segments disclosed herein. Such promoters may be homologous or heterologous promoters, and may be operatively positioned upstream of the nucleic acid segment encoding the therapeutic polypeptide of interest, such that the expression of the segment is under the control of the promoter. The construct may comprise a single promoter, or alternatively, two or more promoters may be used to facilitate expression of the therapeutic gene sequence. Exemplary promoters useful in the practice of the invention include, but are in no way limited to, those promoter sequences that are operable in mammalian, and in particular, human host cells, tissues, and organs, such as for example, a CMV promoter, a β-actin promoter, a hybrid CMV promoter, a hybrid β-actin promoter, an EF1 promoter, a U1a promoter, a U1b promoter, a Tet-inducible promoter and a VP16-LexA promoter being particularly useful in the practice of the invention. In illustrative embodiments, a polynucleotide encoding a therapeutic polypeptide was placed under the control of the chicken β-actin (CBA) promoter and used to produce therapeutically effective levels of the encoded POMC polypeptide when suitable host cells were transformed with the genetic construct.

The polynucleotides comprised in the vectors and viral particles of the present invention may also further optionally comprise one or more native, synthetic, homologous, heterologous, or hybrid enhancer or 5′ regulatory elements, for example, a CMV enhancer, a synthetic enhancer, or an organ- or tissue-specific enhancer operably linked to the therapeutic POMC polypeptide-encoding nucleic acid segments disclosed herein.

The polynucleotides and nucleic acid segments comprised within the vectors and viral particles of the present invention may also further optionally comprise one or more intron sequences. Such sequences may be from native POMC genes, or from other non-related genes.

The POMC-encoding polynucleotides comprised in the vectors and viral particles of the present invention may also further optionally comprise one or more native, synthetic, homologous, heterologous, or hybrid post-transcriptional or 3′ regulatory elements operably positioned relative to the therapeutic polypeptide-encoding nucleic acid segments disclosed herein to provide greater expression, stability, or translation of the encoded polypeptides. One such example is the woodchuck hepatitis virus post-transcriptional regulatory element (WPRE), operably positioned downstream of the therapeutic gene(s) of interest.

In illustrative embodiments, the invention concerns administration of one or more biologically-active pro-opiomelanocortin peptides or polypeptides that comprises, consists essentially of, or consists of, an at least 20, at least 40, at least 60, at least 80, at least 100, at least 120, at least 140, at least 160, at least 180, at least 200, at least 220, at least 240, at least 260, at least 280, at least 300, or more contiguous amino acid sequence from one or more of the polypeptide sequences disclosed hereinbelow and particularly those polypeptides as recited in any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40.

Likewise, in additional illustrative embodiments, the invention concerns administration of one or more biologically-active pro-opiomelanocortin polypeptides that are encoded by a nucleic acid segment that comprises, consists essentially of, or consists of, at least 30, at least 60, at least 90, at least 120, at least 150, at least 180, at least 210, at least 240, at least 270, at least 300, at least 330, at least 360, at least 390, at least 420, at least 450, at least 480, at least 510, at least 540, at least 570, or at least 600, 700, 800, or 900, or more contiguous nucleic acid residues, up to and including substantially full-length, and full-length sequences from the DNA sequences disclosed hereinbelow and particularly nucleic acid segments that comprise, consist essentially of, or consist of one or more of the contiguous nucleotide sequences as recited in any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29.

Exemplary adeno-associated viral vector constructs and polynucleotides of the present invention include those that comprise, consist essentially of, or consist of at least a first nucleic acid segment that encodes a POMC peptide or polypeptide that is at least about 75%, at least about 80%, at least about 85%, at least about 90%, at least about 95%, at least about 96%, at least about 97%, at least about 98%, or at least about 99% identical to the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, wherein the peptide or polypeptide has POMC activity when administered to, and expressed in, a suitable mammalian cell, tissue, or organ.

Exemplary polynucleotides of the present invention also include those sequences that comprise, consist essentially of, or consist of at least a first nucleic acid segment that encodes a polypeptide that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, or SEQ ID NO:40, wherein the peptide or polypeptide encoded by the nucleic acid segment has POMC activity when expressed in a suitable mammalian cell, tissue or organ.

Particularly preferred adeno-associated viral vector constructs and polynucleotides of the present invention include those that comprise, consist essentially of, or consist of at least a first nucleic acid segment that is at least about 75%, at least about 80%, at least about 85%, at least about 90%, at least about 95%, at least about 96%, at least about 97%, at least about 98%, or at least about 99% identical to the sequence of any one of SEQ ID NO: 1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein the segment encodes a peptide or polypeptide that has POMC activity when administered to, and expressed in, a suitable mammalian cell.

Exemplary polynucleotides of the present invention also include those sequences that comprise, consist essentially of, or consist of at least a first nucleic acid segment that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of any one of SEQ ID NO: 1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, or SEQ ID NO:29, wherein the peptide or polypeptide encoded by the nucleic acid segment has POMC activity when expressed in a suitable mammalian cell, tissue, or organ.

### 2.2 RAAV VIRAL PARTICLES AND VIRIONS, AND HOST CELLS COMPRISING THEM

Other aspects of the invention concern rAAV particles and virions that comprise the vectors of the present invention, pluralities of such particles and virions, as well as pharmaceutical compositions and host cells that comprise one or more of the rAAV vectors disclosed herein, such as for example pharmaceutical formulations of the rAAV vectors or virions intended for administration to a mammal through suitable means, such as, by intramuscular, intravenous, or direct injection to selected cells, tissues, or organs of the mammal, for example, to the muscle tissue, the circulatory system, or directly to one or more organs of the selected mammal, such as for example, by direct administration to the liver, or to liver cells. Typically, such compositions will be formulated with pharmaceutically-acceptable excipients, buffers, diluents, adjuvants, or carriers, as described hereinbelow, and may further comprise one or more liposomes, lipids, lipid complexes, microspheres, microparticles, nanospheres, or nanoparticle formulations to facilitate administration to the selected organs, tissues, and cells for which therapy is desired.

Further aspects of the invention include mammalian host cells, and pluralities thereof that comprise one or more of the adeno-associated viral vectors, virions, or viral particles as disclosed herein. Particularly preferred cells are human host cells, and in particular, human bone, blood, liver, pancreatic, kidney, muscle, heart, lung, epithelial, endothelial, or vascular cells.

### 2.3 THERAPEUTIC KITS AND PHARMACEUTICAL COMPOSITIONS

The compositions of the invention also will optionally further comprising at least a first pharmaceutical vehicle, and particularly those formulations that are acceptable for administration to a human through one or more conventional routes of administration, such as for example, oral, nasal, inhalation, transdermal, intravenous, subcutaneous, or intramuscular administration. The compositions of the invention may also further comprise one or more liposomes, lipids, proteins, peptides, polypeptides, nucleic acids, polysaccharides, antibodies, antigens, antigen binding fragments, enzymes, lipid complexes, or at least a first detectable label, marker, or tag. The rAAV particles may be conjugated or otherwise associated with one or more surfaces of a micro- or nanoparticle, such as for example, the nanospheres and microspheres described herein.

Therapeutic kits for treating or ameliorating the symptoms of one or more diseases, dysfunctions, or disorders, or other medical condition resulting from defect, deficiency, or dysfunction of the native POMC polypeptide in a mammal are also part of the present invention. Such kits typically comprise one or more of the disclosed AAV-POMC vector constructs, virion or virus particles, host cells, or therapeutic AAV compositions described herein, and instructions for using the kit.

Another important aspect of the present invention concerns methods of use of the disclosed vectors, virions, compositions, and host cells described herein in the preparation of medicaments for treating or ameliorating the symptoms of one or more diseases, dysfunctions, or disorders, or other medical conditions resulting from defect, deficiency, or dysfunction of the native POMC polypeptide in a mammal. Such methods generally involve administration to a mammal, or human in need thereof, one or more of the disclosed vectors, virions, host cells, or compositions, in an amount and for a time sufficient to treat or ameliorate the symptoms of such a defect, dysfunction, or deficiency in the affected mammal. The methods may also encompass prophylactic treatment of animals suspected of having such conditions, or administration of such compositions to those animals at risk for developing such conditions either following diagnosis, or prior to the onset of symptoms.

Another aspect of the invention concerns compositions that comprise one or more of the disclosed adeno-associated viral vectors, virions, viral particles, and host cells as described herein. Pharmaceutical compositions comprising such are particularly contemplated to be useful in therapy, and particularly in the preparation of medicaments for treating POMC deficiency, dysfunction, or defect in affected mammals, and humans in particular.

The present invention also provides for a host cell that comprises the rAAV-POMC compositions disclosed herein. Preferably, such host cells are mammalian cells, with human host cells being particular preferred. For example, the host cell may be a human pancreas, kidney, muscle, epithelial, endothelial, perivascular, liver, heart, lung, brain, blood, bone, or nerve cell.

### 2.4 THERAPEUTIC METHODS

The invention also provides methods for delivering therapeutically-effective amounts of a biologically-active POMC polypeptide to a mammal in need thereof. Such methods generally comprise at least the step of providing or administering to such a mammal, one or more of the AAV-POMC compositions disclosed herein. For example, the method may involve providing to such a mammal, one or more of the rAAV vectors, virions, viral particles, host cells, or pharmaceutical compositions as described herein. Preferably such providing or such administration will be in an amount and for a time effective to provide a therapeutically-effective amount of one or more of the POMC peptides or polypeptides disclosed herein to selected cells, tissues, or organs of the mammal, and in particular, therapeutically-effective levels to the cells, tissues, or organs of the mammal. Such methods may include systemic injection(s) of the therapeuticum, or may even involve direct or indirect administration, injection, or introduction of the therapeutic compositions to particular cells, tissues, or organs of the mammal, such as for example, by direct injection into muscle or liver tissues.

The invention also provides methods of treating, ameliorating the symptoms, and reducing the severity of POMC deficiency in an animal. These methods generally involve at least the step of providing to an animal in need thereof, one or more of the rAAV vector compositions disclosed herein in an amount and for a time effective to treat the POMC deficiency or other related dysfunction in the animal. As described above, such methods may involve systemic injection(s) of the therapeuticum, or may even involve direct or indirect administration, injection, or introduction of the therapeutic compositions to particular cells, tissues, or organs of the animal.

In one embodiment, the invention provides a method for treating, preventing, or ameliorating the symptoms of a POMC protein, peptide, or polypeptide deficiency or dysfinction in a mammal. The method generally involves administering to a mammal in need thereof, one or more of the disclosed rAAV-POMC vector compositions disclosed herein, in an amount and for a time sufficient to treat, prevent, or ameliorate the symptoms of the POMC deficiency in the mammal. In preferred embodiments, the mammal is a human that is has, is at risk for developing, or has been diagnosed with one or more diseases, disorders, or dysfinctions that result from the deficiency or lack of one or more POMC peptides, polypeptides, or proteins normally present in a healthy subject.

In such cases, the compositions of the invention may be administered to the patient in an amount and for a time sufficient to treat or prevent the symptoms of the POMC deficiency or dysfunction through a single dose, or by administration of a plurality of doses given over a relatively short, or even relatively long period of therapy. The patient may require only one or two administrations of the disclosed rAAV constructs to achieve relatively short-term, relatively medium-term, or even relatively long-term treatment. For example, one or two administrations of the disclosed compositions may provide sufficient therapeutic levels of the POMC composition for a period of several days, several weeks, or several months. Alternatively, three or four administrations of the disclosed compositions either over a relatively short, or relatively long administration period, may provide sufficient therapeutic levels of the POMC composition for a period of several weeks, several months, several years, or even tens of years, up to and including the natural lifetime of the treated mammal.

When relatively short-term therapy is warranted, the therapeutic effectiveness of a single administration or of multiple administrations of the disclosed compositions may persist for a period of about 1, 2, 3, 4, 5, 6, 7, 8, 9, or 10 days or more, and even up to an including a period of about 11, 12, 13, 14, 15, 16, 17, 18, 19, or 20 days or more. When relatively medium-term therapy is warranted, the therapeutic effectiveness of a single administration or of multiple administrations of the disclosed compositions may persist for a period of about 1, 2, 3, 4, 5, 6, 7, 8, 9, or 10 weeks or more, and even up to an including a period of about 11, 12, 13, 14, 15, 16, 17, 18, 19, or 20 weeks or more, such as for example, about 21, about 22, about 23, about 24, about 25, about 26, about 27, about 28, about 29, or about 30 weeks or more, and even up to an including a period of about 31, 32, 33, 34, 35, 36, 37, 38, 39, or 40 weeks or more. Likewise, when relatively long-term therapy is warranted, the therapeutic effectiveness of a single administration or of multiple administrations of the disclosed compositions may persist for a period of about 41, 42, 43, 44, 45, 46, 47, 48, 49, or 50 weeks or more, and even up to an including a period of about 51, 52, 53, 54, 55, 56, 57, 58, 59, or 60, 70, 80, 90, 100, or even 200 or 300 weeks or more. As such, the inventors contemplate that particular therapeutic regimens involving one or more of the compositions disclosed herein will provide a biologically- effective amount of the POMC peptide, polypeptide, or protein, to the individual to which such compositions have been administered, for periods of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, and up to and including periods of therapy that persist in the treated individual for periods of at least about 1 year, at least about 2 years, at least about 3 years, at least about 4 years, year, at least about 6 years, at least about 7 years, at least about 8 years, at least about 9 years, or even at least about 10 or more years, up to and including the natural lifetime of the treated individual.

The rAAV-POMC compositions disclosed herein may be administered by any of the conventional drug delivery methods, such as for example, orally, intranasally, transdermally, intramuscularly, intravenously, subcutaneously, intrathecally, intraperitoneally, or by absorption, inhalation or direct injection into at least a first organ or at least a first tissue of the patient as may be required. Exemplary organs and tissues which may find particular benefit through administration of one or more of the compositions disclosed herein include, but are not limited to, the vascular or circulatory system, the pancreas, liver, heart, lung, brain, kidney, joint, bone, neural, and muscles.

In yet another embodiment, the invention provides kits for treating, preventing, or ameliorating the symptoms of a POMC protein, peptide, or polypeptide-related deficiency or disorder in a mammal, comprising (i) one or more rAAV-POMC composition disclosed herein; and (ii) instructions for using the kit in diagnostic, therapeutic, or prophylactic treatment regimens.

### 3.0 BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1A and FIG. 1B show POMC expression in HEK 293 cells 24 hr after pTR-CBA-POMC or pTR-control transfection. FIG. 1A: Representative image of relative quantitative RT-PCR analysis of POMC MRNA expression with 18S rRNA as an internal standard. FIG. 1B: Quantification of POMC MRNA normalized to 18S rRNA. Data represent mean ±SE from three experiments. *P<0.001 vs. control by unpaired t test.

FIG. 2A and FIG. 2B show hypothalamic POMC expression 38 days after rAAV-POMC or control vector delivery in obese Zucker rats. FIG. 2A: Representative image of relative quantitative RT-PCR analysis of hypothalamic POMC MRNA with 18S rRNA as an internal standard. FIG. 2B: Quantification of POMC mRNA normalized to 18S rRNA. Data represent mean ±SE from six rats per group. *P<0.02 vs. control by unpaired t test.

FIG. 3A and FIG. 3B show total CREB and phosphorylated of CREB (P-CREB) in the hypothalamus of obese Zucker rats 38 days post rAAV-POMC or rAAV-control delivery. FIG. 3A: Representative Western immunoblots using antibodies specific for CREB or P-CREB. FIG. 3B: Quantification of average CREB and P-CREB in six control and six rAAV-POMC rats. Levels in rAAV-control rats are set to 100 and SE adjusted proportionally. *P <0.05 vs. control by unpaired t test.

FIG. 4A shows body weight gain, FIG. 4B shows daily food consumption, and FIG. 4C shows percentage of initial food intake after rAAV-POMC or rAAV-control administration in obese Zucker rats. The vectors were injected at day 0. Data represent mean +SE of six rats per group. Initial food intake is the average daily food consumption one week before vector delivery. P<0.0001 for difference in weight gain (FIG. 4A), food intake (FIG. 4B) or percentage of initial food intake (FIG. 4C) with treatment by repeated measures ANOVA. P<0.05 for difference in food intake between rAAV-POMC and control rats at day 7 through day 38 except day 35.

FIG. 5A shows visceral adiposity and FIG. 5B shows fasting serum leptin 38 days after rAAV-POMC or rAAV-control administration in obese Zucker rats. Data represent mean ±SE of six rats per group. *P<0.05 vs. control by unpaired t test.

FIG. 6A shows fasting serum insulin, FIG. 6B shows glucose, FIG. 6C shows cholesterol and FIG. 6D shows FFA levels 38 days after rAAV-POMC or rAAV-control delivery in obese Zucker rats. Data represent mean +SE of six rats per group. * P<0.05 and †P<0.01 vs. control by unpaired t test.

FIG. 7 shows a diagram of an illustrative rAAV vector plasmid. TR is AAV2 terminal repeat sequence; CBA promoter includes the CMV intermediate early enhancer sequence, the chicken β-actin promoter, non-coding sequence (Exoni) and intron from rabbit β-globin gene; the murine POMC; WPRE is the woodchuck hepatitis virus post-transcription regulatory sequence; bGH poly(A) is the bovine growth hormone polyadenylation sequence.

FIG. 8A and FIG. 8B show immunohistochemical localization of GFP in rats given unilateral injection of rAAV-control. FIG. 8A shows numerous GFP-positive cells are observed in a representative coronal hypothalamic section following rAAV-control delivery. The highest density of GFP immunoreactivity is found in the arcuate nucleus (ARC) (4×). FIG. 8B shows the same hypothalamic section at a higher magnification (10×). 3v=third ventricle.

FIG. 9A and FIG. 9B show hypothalamic POMC expression 42 days after rAAV-POMC or control vector delivery in aged-obese rats. FIG. 9A is a representative image of relative quantitative RT-PCR™ analysis of hypothalamic POMC mRNA with 18S rRNA as an internal standard. FIG. 9B shows quantification of POMC mRNA normalized to 18S rRNA. Data represent mean ±SE from 6 rats per group. *P<0.001 vs. control by unpaired t test.

FIG. 10A and FIG. 10B are graphs showing food consumption (FIG. 10A) and body weight change (FIG. 10B) following rAAV-POMC or rAAV-control administration in aged obese rats. The vectors were injected at day 0. Data represent mean ±SE of 6 rats per group. P<0.001 for difference in food intake (FIG. 10A) and weight change (FIG. 10B) with treatment by repeated measures ANOVA. P<0.05 for difference in food intake (FIG. 10A) and weight change (FIG. 10B) between rAAV-POMC and control rats at day 10 through day 27 and at day 10 through day 42, respectively.

FIG. 11A and FIG. 11B are graphs showing visceral adiposity (FIG. 1lA) and fasting serum leptin (FIG. 11B) 42 days following rAAV-POMC or rAAV-control administration in aged obese rats. Data represent mean ±SE of 6 rats per group. *P<0.05 vs. control by unpaired t test.

FIG. 12A and FIG. 12B are graphs showing blood glucose (FIG. 12A) and plasma insulin (FIG. 12B) levels during intraperitoneal glucose tolerance test in aged obese rats. Data represent mean ±SE of 6 rats per group. P<0.05 for difference in blood glucose (FIG. 12A) and plasma insulin (FIG. 12B) with treatment by repeated measures ANOVA. *P<0.05 and tP<0.01 vs. control by unpaired t test.

FIG. 13A, FIG. 13B and FIG. 13C are graphs showing serum FFA (FIG. 13A), triglyceride (FIG. 13B), and cholesterol (FIG. 13C) levels 42 days following rAAV-POMC or rAAV-control delivery in aged obese rats. Data represent mean ±SE of 6 rats per group. *P<0.05 vs. control by unpaired t test.

### 4.0 DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### 4.1 OBESITY

Obesity is a complex disorder and often leads to hyperinsulinemia, hyperglycemia and insulin resistance. Obesity is also a major risk factor for hypertension and cardiovascular disease. There are multiple pathways controlling the complex balance of energy intake and expenditure. The major afferent factor in a negative feedback loop regulating daily food intake and body weight is hormone leptin synthesized in adipocytes. Although leptin administration has been shown to reduce food intake and body weight in rodents its effectiveness is only transient and requires repeated injections. In humans, plasma levels of leptin increase in direct correlation with increase in body weight and adiposity; this tolerance to leptin (leptin resistance) is believed to be an underlying factor in the loss of leptin control on energy balance. In addition, leptin-resistance due to environmental and genetic factors contributes substantially to human obesity. The cellular and molecule mechanisms of leptin resistance are not known. It is believed that leptin resistance is heterogeneous and multiple factors, including defective transport to brain and defective influence on the activity of the neural circuits that regulate body weight, are major players. Consequently, there is a clear need to develop innovative approaches to control appetite and body weight and to correct and manage obesity.

### 4.2 ADENO-ASSOCIATED VIRUS AND EXPRESSION VECTORS

Adeno-associated virus (AAV) is a single-stranded DNA parvovirus with a 4.7 kb genome and a particle diameter of approximately 20 nm. The AAV genome is flanked by two identical inverted terminal repeat (ITR) sequences (Lusby et al., 1980). These ITRs provide all the cis-acting sequence required for replication, packaging and integration (Samulski et al., 1989). There are two large open reading frames (Srivastava et al., 1983). The open reading frame in the right half of the genome (cap) encodes 3 overlapping coat proteins (VP1, VP2 and VP3). The open reading frame in the left half (rep gene) encodes 4 regulatory proteins with overlapping sequences which are known as Rep proteins (Rep78, Rep68, Rep52 and Rep40), because frame shift mutations at most locations within the open reading frame inhibit viral DNA replication (Hermonat et al., 1984). The Rep proteins are multi-finctional DNA binding proteins. The functions of the Rep proteins in viral DNA replication include helicase activity and a site-specific, strand-specific endonuclease (nicking) activity (Ni et al., 1994).

AAV infects a broad spectrum of vertebrates from birds to humans, although in nature specific types are species specific (B ems, 1996). In humans AAV can infect a large variety of cells derived from different tissues. The infection of AAV is ubiquitous within the population with about 90% of adults being seropositive (Cukor et al., 1983). In spite of its omnipresence, AAV has never been associated with any human disease. In this sense, rAAV is the safest of the currently used gene therapy vectors.

Because of its propensity to establish latency and because it has not been implicated as a pathogen, AAV has been of considerable interest as a potential vector for human gene therapy (Flotte and Ferkol, 1997; Flotte and Carter, 1995). In general, rAAV vectors are produced by deleting the viral coding sequences and substituting the transgene of interest under control of a non-AAV promoter between the two AAV inverted terminal repeats (ITRs). When the rep and cap proteins are expressed in trans in Ad-infected cells, rAAV genomes can be efficiently packaged. Considerations in the development of AAV as a vector have included difficulties in attaining high vector titers and the limited insertional capacity (>5 kb). Although these issues can still be improved, recently developed packaging techniques for high titer and Ad-contamination free vectors, and strategies to overcome the packaging limitation, have dramatically impacted the applications of rAAV (Zolotukhin et al., 1999; Duan et al., 2000; Yan et al., 2000). Unlike adenovirus vectors, rAAV vectors are remarkably nonimmunogenic with little host response (Jooss et al., 1998; Song et al., 1998). In addition to the above unique features, rAAV have mediated long-term transgene expression in a wide variety of tissues, including muscle (Song et al., 1998; Kessler et al., 1996; Xiao et al., 1996; Clark et al., 1997; Snyder et al., 1997a), lung (Flotte et al., 1993), liver (Snyder et al., 1997b; Xiao et aL, 1998; Song et al., 2001a; Xu et al., 2001), brain (Kaplitt et al., 1994) and eye (Flannery et al., 1997). Thus rAAV vectors appear to have significant advantages over other commonly used viral vectors.

Six serotypes of AAV have been cloned and sequenced. Of the six AAV serotypes, serotype 2 (AAV2) is the best-characterized and has been predominantly used in gene transfer studies. Membrane-associated heparan sulfate proteoglycan is the primary receptor for AAV type 2 (Summerford and Samulski, 1998). Human fibroblast growth factor receptor 1 and αᵥβ₅ integrin are co-receptors for AAV2 (Qing et al., 1999; Summerford et al., 1999). Serotypes 1 and 6 share >99% amino acid homology in their capsid proteins. Sequence analysis supports a recombination event between seroType I and 2. Comparison of the serotype capsid amino acid sequences suggests that serotypes, 1, 2, and 3 share homology across the three capsids in accord with heparan sulfate binding (Summerford and Samulski, 1998). In contrast, AAV type 4 and 5 are the most divergent of the six AAV serotypes, exhibiting only 60% homology to AAV2 or to each other. AAV4 and AAV5 require different sialic acid-containing glycoproteins for binding and transduction of target cells. The different tropisms of AAV serotypes provide opportunities to optimize the transduction efficiency in different target cells. Data showed that of the serotypes, AAV1 mediated the highest transgene expression in skeletal muscle and murine islets (Chao et al., 2000).

The AAV life cycle is quite unusual (Bems and Linden, 1995). AAV binds to cells via a heparan sulfate proteoglycan receptor (Summerford and Samulski, 1998). Once attached, AAV entry is dependent upon the presence of a co-receptor, which may consist of either the fibroblast growth factor receptor (FGF-R) (Qing et al., 1999) or the αᵥ-β₅ integrin molecule (Summerford et al., 1999). Cells infected with AAV and a helper virus (or another adjunctive agent, such as UV irradiation or treatment with genotoxic agents) will undergo productive replication of AAV prior to cell lysis, which is induced by the helper rather than by AAV. Human cells infected with AAV alone, however, will become persistently infected (Berns et al., 1975). This latency pathway often results in colinear integration of AAV sequences within the host cell genome (Cheung et al., 1980), often within a specific site on human chromosome 19, the AAVS1 site (Kotin et al., 1990; Kotin et al., 1991; Kotin et aL, 1992; Samulski et al., 1991; Samulski, 1993). While this site is not strictly homologous to AAV, it contains consensus elements required for binding and nicking by the AAV Rep protein, a non-structural protein that is also involved in productive replication and in transcriptional regulation of the virus (Weitzman et al., 1994; Giraud et al., 1994; Giraud et al., 1995; Linden et al., 1996). Once AAV is integrated, it will remain stable within infected cells for prolonged periods of time, up to 100 passages (Hoggan et al., 1972). Episomal forms of the virus may also be present for extended periods in some circumstances (Afione et al., 1996; Kearns et al., 1996; Flotte et al., 1994). If latently infected cells are subsequently infected with a helper virus, the genome will be excised and productive AAV replication and packaging will ensue (Senapathy et aL, 1984; Afione et al., 1996).

The AAV genome consists of two 145-nucleotide inverted terminal repeat (ITR) sequences, each an identical palindrome at either terminus of the virus, flanking the two AAV genes, rep and cap (Tratschin et al., 1984a). The rep gene is transcribed from two promoters, the p5 promoter (at map position 5) and the p19 promoter (map position 19), which is embedded within the coding sequence of the longer Rep proteins. In each case, both the spliced and unspliced transcripts are processed and translated. This allows for the production of 4 Rep proteins, Rep78, Rep68, Rep52, and Rep4O. Rep78 and Rep68 are multifunctional DNA binding proteins which possess helicase activity and site-specific, strand-specific nickase activity, both of which are required for terminal resolution of replicating AAV genomes (Im and Muzyczka, 1990). The long Rep proteins are also capable of binding to the chromosomal target sequence for AAV integration, the AAVS1 site, and these proteins are required for normal integration into this site. Finally, Rep78/68 are potent bi-functional transcription regulators that generally activate transcription from AAV promoters during productive infection and repress their transcription during latent infection (Pereira and Muzyczka, 1997; Pereira et al., 1997). The shorter Rep proteins, Rep52 and Rep40 act as modifier proteins for long Rep transcriptional activities, and are required for sequestration of single-stranded AAV genomes into capsids during productive infection.

The AAV cap gene is transcribed from the p40 promoter. The 5′ end of the mRNA transcript from p40 contains an intron which can utilize either of two downstream splice acceptor sites. The longer of the two processed messages contains an ATG codon which is used in the translation of VP1, the longest of the three AAV capsid proteins. The shorter MRNA can initiate translation using either a non-canonical ACG start codon, which represents the start of VP2, or an ATG codon further downstream, which comprises the N-terminal Met of VP3 (Trempe and Carter, 1988). VP3 is the shortest and most abundant of the AAV capsid proteins, but all three are required for the production of infectious virions.

### 4.3 RECOMBINANT AAV VECTORS

Recombinant AAV (rAAV) vectors have been developed by replacement of the viral coding sequences with transgene of interest (Tratschin et al., 1984a; Hermonat and Muzyczka, 1984). The ITR sequences must be retained in rAAV since these serve as origins for viral DNA replication and contain the packaging signals. The maximum packaging capacity of rAAV is approximately 5 kb, including the ITRs, the transgene, its promoter, and polyadenylation signal (Flotte et al., 1992; Dong et al., 1996). The full exploitation of rAAV for gene transfer has been limited in the past primarily by the packaging and purification process. In particular, contamination of rAAV vector preparations with wild-type AAV has been found to alter the biological behavior of the vector, and limitations on the titers and infectivity of the vectors have limited their widespread use on the past. Recent advances in the packaging and purification technology have resulted in a dramatic improvement in the expression levels that have been achievable in vivo. In particular, the use of adenoviral plasmids and of complementing rep gene expression constructs that express less of the longer Rep proteins (Rep68/78) has resulted in a substantial improvement in the efficiency of vector production on a per cell basis (Xiao et al., 1998; Li et al., 1997). The availability of packaging cell lines has also resulted in a substantial improvement in the scale-ability of the packaging process (Clark et al., 1996; Flotte et al., 1995; Gao et al., 1998). Finally, the availability of several column chromatography methods, including heparin sulfate affinity column chromatography, has allowed for the elimination of CsCl banding, which in turn appears to have enhanced the infectivity of output particles (Zolotukhin et al., 1999).

rAAV vectors are uniquely suitable for in vivo gene therapy for genetic and metabolic disorders, since they are non-toxic (Flotte et al., 1993; Conrad et al., 1996; Flotte and Carter, 1998), highly efficient when used at high titers, relatively non-immunogenic (Jooss et al., 1998; Hernandez et al., 1999; Beck et al., 1999), and very stable in their pattern of expression. The utility of rAAV vectors for in vitro and in vivo gene transfer has now been well established. There appear to be important tissue specific differences in rAAV effects, however. rAAV vectors have been found to be particularly efficient for gene transfer into terminally differentiated cells such as neurons (Kaplitt et al., 1994; McCown et al., 1996; Peel et al., 1997; Mandel et al., 1997), myofibers (Xiao et al., 1996; Kessler et al., 1996; Clark et al., 1997; Fisher et al., 1997; Song et al., 1998), and photoreceptor cells (Flannery et al., 1997; Lewin et al., 1998; Zolotukhin et al., 1996; Rolling et al., 1999). Gene transfer to the bronchial epithelium has also been demonstrated (Flotte et al., 1993; Conrad et aL, 1996; Afione et al., 1996; Flotte et al., 1998; Halbert et al., 1998), although the efficiency of transduction remains relatively low. Likewise, rAAV transduction of hepatocytes has also been studied, and has been found to be efficient enough to provide a potential therapeutic strategy for hemophilia B, by providing persistent and therapeutic concentrations of human Factor IX in mice (Snyder et al., 1997b). However, in that study, in situ hybridization results indicated that only 5% of hepatocytes had been transduced (Miao et al., 1998).

In the case of each of these two cell types, recent evidence has shown that the efficiency can be enhanced by altering the capsid to incorporate ligands for a receptor that is abundantly expressed on the cell surface and by optimizing the promoter usage (Wu et al., 2000; Virella-Lowell et al., 1999). Similar manipulations are also advantageous in pancreatic islet cells. Recent reports of severe dose-related clinical adverse events due to adenovirus, although not directly reflective of rAAV, underscore the necessity of minimizing the dose of vector whenever possible.

### 4.4 RAAV THERAPY FOR HUMAN DISEASES

During recent years, viral vector-based human gene therapy approaches have been developed as potentially effective, alternative treatment modalities for a variety of diseases. In particular, adeno-associated virus 2 (AAV), a defective parvovirus of human origin, has been demonstrated to be one such promising vector. AAV is an ideal vector for viral-based human gene therapy because it has not been associated with any known pathology and post-infection, the viral genome integrates into the human chromosome (Muzyczka, 1992). Recombinant adeno-associated virus (rAAV) vectors have important utility as vehicles for the in vivo delivery of polynucleotides to target host cells (Kessler et al., 1996; Koeberl et al., 1997; Kotin, 1994; Xiao et al., 1996). rAAV vectors are useful vector for efficient and long-term gene transfer in a variety of mammalian tissues, e.g., lung (Flotte et al., 1993), muscle (Kessler et al., 1996; Xiao et aL, 1996; Clark et al., 1997; Fisher et al., 1997), brain (Kaplitt et al., 1994; Klein et al., 1998) retina (Flannery et al., 1997; Lewin et al., 1998), and liver (Snyder et al., 1997s).

It has also been shown that rAAV can evade the immune response of the host by failing to transduce dendritic cells (Jooss et al., 1998). Clinical trials have been initiated for several important mammalian diseases including hemophilia B, muscular dystrophy and cystic fibrosis (Flotte et al., 1996; Wagner et al., 1998; Flotte and Carter, 1995; Kay et al., 2000). As with most gene therapy vectors, obstacles in the efficient use of rAAV vectors for a variety of disease models include sub-therapeutic levels of transduction and the ability to target the site(s) of gene transfer.

### 4.5 PROMOTERS AND ENHANCERS

Recombinant vectors form important aspects of the present invention. The term “expression vector or construct” means any type of genetic construct containing a nucleic acid in which part or all of the nucleic acid encoding sequence is capable of being transcribed. In preferred embodiments, expression only includes transcription of the nucleic acid, for example, to generate a pro-opiomelanocortin polypeptide product from a transcribed gene.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively linked,” “operably linked,” “operatively positioned,” “under the control of” or “under the transcriptional control of” means that the promoter is in the correct location and orientation in relation to the nucleic acid segment that comprises the therapeutic gene to properly facilitate, control, or regulate RNA polymerase initiation and expression of the therapeutic gene to produce the therapeutic polypeptide in the cells that comprise the genetic construct.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a pro-opiomelanocortin-encoding gene in its natural environment. Such promoters may include promoters normally associated with other genes, and/or promoters isolated from any other bacterial, viral, eukaryotic, or mammalian cell.

Naturally, it will be important to employ a promoter that effectively directs the expression of the pro-opiomelanocortin-encoding DNA segment in the cell type, organism, or even animal, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high-level expression of the introduced DNA segment.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter, such as a beta-actin, CMV or HSV promoter. In certain aspects of the invention, inducible promoters, such as tetracycline-controlled promoters, are also contemplated to be useful in certain cell types.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. Tables 1 and 2 below list several elements/promoters that may be employed, in the context of the present invention, to regulate the expression of the pro-opiomelanocortin-encoding constructs. This list is not intended to be exhaustive of all the possible elements involved in the promotion of transgene expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER AND ENHANCER ELEMENTS

| PROMOTER/ENHANCER | REFERENCES |
|---|---|
| Immunoglobulin Heavy Chain | Banerji et al., 1983; Gilles et al., 1983; Grossched1 and |
| | Baltimore, 1985; Atchison and Perry, 1986, 1987; Imler |
| | et al., 1987; Weinberger et al., 1984; Kiledjian et al., |
| | 1988; Porton et al.; 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo |
| | et al.; 1990 |
| HLA DQ a and DQ β | Sullivan and Peterlin, 1987 |
| β-Interferon | Goodbourn et al., 1986; Fujita et al., 1987; Goodbourn |
| | and Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick and Benfield, 1989; Johnson |
| | et al., 1989 |
| Prealbumin (Transthyretin) | Costa et al., 1988 |
| Elastase I | Ornitz et al., 1987 |
| Metallothionein | Karin et al., 1987; Culotta and Hamer, 1989 |
| Collagenase | Pinkert et al., 1987; Angel et al., 1987a |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | Cohen et al., 1987 |
| c-HA-ras | Treisman, 1986; Deschamps et al., 1985 |
| Insulin | Edlund et al., 1985 |
| Neural Cell Adhesion Molecule | Hirsh et al., 1990 |
| (NCAM) | |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and |
| | Lockett, 1985; Firak and Subramanian, 1986; Herr and |
| | Clarke, 1986; Imbra and Karin, 1986; Kadesch and Berg, |
| | 1986; Wang and Calame, 1986; Ondek et al., 1987; Kuhl |
| | et al., 1987; Schaffner et al., 1988 |
| Polyoma | Swartzendruber and Lehman, 1975; Vasseur et al., 1980; |
| | Katinka et al., 1980, 1981; Tyndall et al., 1981; Dandolo |
| | et al., 1983; de Villiers et al., 1984; Hen et al., 1986; |
| | Satake et al., 1988; Campbell and Villarreal, 1988 |
| Retroviruses | Kriegler and Botchan, 1982, 1983; Levinson et al., 1982; |
| | Kriegler et al., 1983, 1984a, b, 1988; Bosze et al., 1986; |
| | Miksicek et al., 1986; Celander and Haseltine, 1987; |
| | Thiesen et al., 1988; Celander et al., 1988; Choi et al., |
| | 1988; Reisman and Rotter, 1989 |
| Papilloma Virus | Campo et al., 1983; Lusky et al., 1983; Spandidos and |
| | Wilkie, 1983; Spalholz et al., 1985; Lusky and Botchan, |
| | 1986; Cripe et al., 1987; Gloss et al., 1987; Hirochika |
| | et al., 1987; Stephens and Hentschel, 1987 |
| Hepatitis B Virus | Bulla and Siddiqui, 1986; Jameel and Siddiqui, 1986; |
| | Shaul and Ben-Levy, 1987; Spandau and Lee, 1988; |
| | Vannice and Levinson, 1988 |
| Human Immunodeficiency Virus | Muesing et al., 1987; Hauber and Cullen, 1988; |
| | Jakobovits et al., 1988; Feng and Holland, 1988; Takebe |
| | et al., 1988; Rosen et al., 1988; Berkhout et al., 1989; |
| | Laspia et al., 1989; Sharp and Marciniak, 1989; Braddock |
| | et al., 1989 |
| Cytomegalovirus | Weber et al., 1984; Boshart et al., 1985; Foecking and |
| | Hofstetter, 1986 |
| Gibbon Ape Leukemia Virus | Holbrook et al., 1987; Quinn et al., 1989 |

TABLE 2
INDUCIBLE ELEMENTS

| ELEMENT | INDUCER | REFERENCES |
|---|---|---|
| MT II | Phorbol Ester (TFA) | Palmiter et al., 1982; Haslinger |
| | Heavy metals | and Karin, 1985; Searle et al., |
| | | 1985; Stuart et al., 1985; |
| | | Imagawa et al., 1987, Karin et al., |
| | | 1987; Angel et al., 1987b; |
| | | McNeall et al., 1989 |
| MMTV (mouse mammary | Glucocorticoids | Huang et al., 1981; Lee et al., |
| tumor virus) | | 1981; Majors and Varmus, 1983; |
| | | Chandler et al., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2 κb | Interferon | Blanar et al., 1989 |
| HSP70 | Ela, SV40 Large T Antigen | Taylor et al., 1989; Taylor and |
| | | Kingston, 1990a, b |
| Proliferin | Phorbol Ester-TPA | Mordacq and Linzer, 1989 |
| Tumor Necrosis Factor | FMA | Hensel et al., 1989 |
| Thyroid Stimulating | Thyroid Hormone | Chatterjee et al., 1989 |
| Hormone a Gene | | |

As used herein, the terms “engineered” and “recombinant” cells are intended to refer to a cell into which an exogenous DNA segment, such as DNA segment that leads to the transcription of a pro-opiomelanocortin polypeptide, has been introduced. Therefore, engineered cells are distinguishable from naturally occurring cells, which do not contain a recombinantly introduced exogenous DNA segment. Engineered cells are thus cells having DNA segment introduced through the hand of man.

To express a pro-opiomelanocortin gene in accordance with the present invention one would prepare an rAAV expression vector that comprises a pro-opiomelanocortin-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constructs are those that comprise an rAAV vector. Such vectors are described in detail herein.

### 4.6 PHARMACEUTICAL COMPOSITIONS

In certain embodiments, the present invention concerns formulation of one or more of the rAAV compositions disclosed herein in pharmaceutically acceptable solutions for administration to a cell or an animal, either alone, or in combination with one or more other modalities of therapy.

It will also be understood that, if desired, the nucleic acid segment, RNA, DNA or PNA compositions that express a therapeutic gene product as disclosed herein may be administered in combination with other agents as well, such as, e.g., proteins or polypeptides or various pharmaceutically-active agents, including one or more systemic or topical administrations of pro-opiomelanocortin polypeptides. In fact, there is virtually no limit to other components that may also be included, given that the additional agents do not cause a significant adverse effect upon contact with the target cells or host tissues. The rAAV-pro-opiomelanocortin compositions may thus be delivered along with various other agents as required in the particular instance. Such compositions may be purified from host cells or other biological sources, or alternatively may be chemically synthesized as described herein. Likewise, such compositions may further comprise substituted or derivatized RNA, DNA, or PNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Solutions of the active compounds as freebase or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

### 4.7 LIPOSOME-, NANOCAPSULE-, AND MICROPARTICLE-MEDIATED DELIVERY

In certain embodiments, the inventors contemplate the use of liposomes, nanocapsules, microparticles, microspheres, lipid particles, vesicles, and the like, for the introduction of the compositions of the present invention into suitable host cells. In particular, the rAAV vector compositions of the present invention may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV-pro-opiomelanocortin constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e., in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; Couvreur, 1988; zur Muhlen et al., 1998; Zambaux et al. 1998; Pinto-Alphandry et al., 1995 and U.S. Pat. No. 5,145,684, specifically incorporated herein by reference in its entirety).

### 4.8 ADDITIONAL MODES OF DELIVERY

In addition to the methods of delivery described above, the following techniques are also contemplated as alternative methods of delivering the disclosed rAAV vector-based polynucleotide compositions to target cells or selected tissues and organs of an animal, and in particular, to cells, organs, or tissues of a vertebrate mammal, and more particularly, to a primate, such as a human being. Sonophoresis (i.e., ultrasound) has been used and described in U.S. Pat. No. 5,656,016 (specifically incorporated herein by reference in its entirety) as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. No. 5,770,219 and U.S. Pat. No. 5,783,208) and feedback-controlled delivery (U.S. Pat. No. 5,697,899), each specifically incorporated herein by reference in its entirety.

### 4.9 THERAPEUTIC AND DIAGNOSTIC KITS

The invention also encompasses one or more compositions together with one or more pharmaceutically-acceptable excipients, carriers, diluents, adjuvants, and/or other components, as may be employed in the formulation of particular rAAV-polynucleotide delivery formulations, and in the preparation of therapeutic agents for administration to a mammal, and in particularly, to a human, for one or more of the pro-opiomelanocortin-deficient conditions described herein. In particular, such kits may comprise one or more rAAV-pro-opiomelanocortin composition in combination with instructions for using the viral vector in the treatment of such disorders in a mammal, and may typically further include containers prepared for convenient commercial packaging.

As such, preferred animals for administration of the pharmaceutical compositions disclosed herein include mammals, and particularly humans. Other preferred animals include murines, bovines, equines, porcines, canines, and felines. The composition may include partially or significantly purified rAAV-pro-opiomelanocortin compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the compositions disclosed herein and instructions for using the composition as a therapeutic agent. The container means for such kits may typically comprise at least one vial, test tube, flask, bottle, syringe or other container means, into which the disclosed rAAV composition(s) may be placed, and preferably suitably aliquoted. Where a second pro-opiomelanocortin composition is also provided, the kit may also contain a second distinct container means into which this second composition may be placed. Alternatively, the plurality of pro-opiomelanocortin compositions may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

### 4.10 METHODS OF NUCLEIC ACID DELIVERY AND DNA TRANSFECTION

In certain embodiments, it is contemplated that one or more of the rAAV-delivered pro-opiomelanocortin-encoding RNA, DNA, PNAs and/or substituted polynucleotide compositions disclosed herein will be used to transfect an appropriate host cell. Technology for introduction of rAAVs comprising one or more PNAs, RNAs, and DNAs into target host cells is well known to those of skill in the art.

Several non-viral methods for the transfer of expression constructs into cultured mammalian cells also are contemplated by the present invention for use in certain in vitro embodiments, and under conditions where the use of rAAV-mediated delivery is less desirable. These include calcium phosphate precipitation (Graham and van der Eb, 1973; Chen and Okayama, 1987; Rippe et al., 1990) DEAE-dextran (Gopal, 1985), electroporation (Wong and Neumann, 1982; Fromm et al., 1985; Tur-Kaspa et al., 1986; Potter et al., 1984; Suzuki et al., 1998; Vanbever et al., 1998), direct microinjection (Capecchi, 1980; Harland and Weintraub, 1985), DNA-loaded liposomes (Nicolau and Sene, 1982; Fraley et al., 1979; Takakura, 1998) and lipofectamine-DNA complexes, cell sonication (Fechheimer et al., 1987), gene bombardment using high velocity microprojectiles (Yang et al., 1990; Klein et al., 1992), and receptor-mediated transfection (Curiel et al., 1991; Wagner et al., 1992; Wu and Wu, 1987; Wu and Wu, 1988). Some of these techniques may be successfully adapted for in vivo or ex vivo use.

### 4.11 EXPRESSION IN ANIMAL CELLS

The inventors contemplate that a polynucleotide comprising a contiguous nucleic acid sequence that encodes a therapeutic pro-opiomelanocortin polypeptide of the present invention may be utilized to treat one or more cellular defects in a host cell that comprises the vector. Such cells are preferably animal cells, including mammalian cells such as those obtained from a human or other primates, murine, canine, feline, bovine, equine, epine, or porcine species. In particular, the use of such constructs for the treatment and/or amelioration of eating disorders or neurological dysfunction in a human subject suspected of suffering from such a disorder is contemplated. The cells may be transformed with one or more rAAV vectors comprising one or more therapeutic pro-opiomelanocortin genes of interest, such that the genetic construct introduced into and expressed in the host cells of the animal is sufficient to alter, reduce, ameliorate or prevent the deleterious or disease conditions either in vitro and/or in vivo.

### 4.12 TRANSGENIC ANIMALS

It is contemplated that in some instances the genome of a transgenic non-human animal will have been altered through the stable introduction of one or more of the rAAV-delivered polynucleotide compositions described herein, either native, synthetically modified, or mutated. As used herein, the term “transgenic animal” is intended to refer to an animal that has incorporated exogenous DNA sequences into its genome. In designing a heterologous gene for expression in animals, sequences which interfere with the efficacy of gene expression, such as polyadenylation signals, polymerase II termination sequences, hairpins, consensus splice sites and the like are eliminated. Current advances in transgenic approaches and techniques have permitted the manipulation of a variety of animal genomes via gene addition, gene deletion, or gene modifications (Franz et al., 1997). For example, mosquitoes (Fallon, 1996), trout (Ono et al., 1997), zebrafish (Caldovic and Hackett, 1995), pigs (Van Cott et al., 1997) and cows (Haskell and Bowen, 1995), are just a few of the many animals being studied by transgenics. The creation of transgenic animals that express human proteins such as α-1-antitrypsin, in sheep (Carver et al., 1993); decay accelerating factor, in pigs (Cozzi et al., 1997), and plasminogen activator, in goats (Ebert et al., 1991) has previously been demonstrated. The transgenic synthesis of human hemoglobin (U.S. Pat. No. 5,602,306) and fibrinogen (U.S. Pat. No. 5,639,940) in non-human animals have also been disclosed, each specifically incorporated herein by reference in its entirety. Further, transgenic mice and rat models have recently been described as new directions to study and treat cardiovascular diseases such as hypertension in humans (Franz et al., 1997; Pinto-Siestma and Paul, 1997). The construction of a transgenic mouse model has recently been used to assay potential treatments for Alzheimer's disease (U.S. Pat. No. 5,720,936, specifically incorporated herein by reference in its entirety). It is contemplated in the present invention that transgenic animals contribute valuable information as models for studying the effects of pro-opiomelanocortin compositions on correcting genetic defects and treating a variety of disorders in an animal.

### 4.13 SELECTION AND CHARACTERIZATION OF PRO-OPIOMELANOCORTIN GENETIC CONSTRUCTS

The enzyme luciferase is useful as a screenable marker in the context of the present invention (Kang et al., 1998). In the presence of the substrate luciferin, cells expressing luciferase emit light that can be detected on photographic or x-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells that are expressing luciferase and manipulate those in real time. The above techniques also could be utilized if the screenable marker is a protein such as green fluorescent protein (gfp).

To confirm the presence of the exogenous DNA or “transgene(s)” in the transformed cells, and in particular, a transgene delivered by an rAAV vector composition, a variety of assays may be performed. Such assays include, for example, “molecular biological” assays, such as Southern and Northern blotting, RT-PCRTM and PCRTM; “biochemical” assays, such as detecting the presence of a protein product, e.g., by immunological means (ELISAs and Western blots) or by enzymatic function assay.

While Southern blotting and PCRTM may be used to detect the transgene(s) in question, they do not provide information as to whether the gene is being expressed. Expression may be evaluated by RT-PCRTM for mRNA and/or specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Transgenic animals are described that synthesize epitope tagged prion proteins as a method of detecting the expressed protein(s) (U.S. Pat. No. 5,789,655, specifically incorporated herein by reference in its entirety). Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including but not limited to analyzing changes in the chemical composition, morphology, or physiological properties of the cells of the animal or human.

### 4.14 DNA INTEGRATION, RNA EXPRESSION AND INHERITANCE

Genomic DNA may be isolated from animal cell lines or any animal parts to determine the presence of the exogenously introduced pro-opiomelanocortin-encoding genetic construct through the use of one or more readily-available techniques that are well known to those skilled in the art. The presence of DNA elements introduced through the methods of this invention may be determined by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. In addition, it is not possible using PCR™ techniques to determine whether transformants have exogenous genes introduced into different sites in the genome, i.e., whether transformants are of independent origin. It is contemplated that using PCRTM techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™ e.g., the presence of a gene, but also demonstrates integration into the genome and characterizes each individual transformant.

It is contemplated that using the techniques of dot or slot blot hybridization which are modifications of Southern hybridization techniques one could obtain the same information that is derived from PCR™, e.g., the presence of a gene.

Whereas DNA analysis techniques may be conducted using DNA isolated from any part of an animal, RNA will only be expressed in particular cells or tissue types and hence it will be necessary to prepare RNA for analysis from these tissues. PCR™ techniques may also be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridization. These techniques are modifications of Northern blotting and will only demonstrate the presence or absence of an RNA species.

### 4.15 SELECTABLE MARKERS

In certain embodiments of the invention, the delivery of a nucleic acid in a cell, and in particular, an rAAV construct that expresses one or more therapeutic pro-opiomelanocortin compositions may be identified in vitro or in vivo by including a marker in the expression construct. The marker would result in an identifiable change to the transfected cell permitting ready identification of expression. Usually the inclusion of a drug selection marker aids in cloning and in the selection of transformants, for example, neomycin, puromycin, hygromycin, DHFR, GPT, zeocin and histidinol. Alternatively, enzymes such as herpes simplex virus thymidine kinase (tk) (eukaryotic) or chloramphenicol acetyltransferase (CAT) (prokaryotic) may be employed, as well as markers such as green fluorescent protein, luciferase, and the like. Immunologic markers also can be employed. The selectable marker employed is not believed to be important, as long as it is capable of being expressed simultaneously with the nucleic acid encoding a gene product. Further examples of selectable markers are well known to one of skill in the art.

### 4.16 SITE-SPECIFIC MUTAGENESIS

Site-specific mutagenesis is a technique useful in the preparation of individual peptides, or biologically functional equivalent polypeptides, through specific mutagenesis of the underlying polynucleotides that encode them. The technique, well-known to those of skill in the art, further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or change the properties of the polynucleotide itself, and/or alter the properties, activity, composition, stability, or primary sequence of the encoded polypeptide.

In certain embodiments of the present invention, the inventors contemplate the mutagenesis of the disclosed pro-opiomelanocortin-encoding polynucleotide sequences to alter the activity or effectiveness of such constructs in increasing or altering their therapeutic activity in a transformed host cell. Likewise in certain embodiments, the inventors contemplate the mutagenesis of such genes themselves, or of the rAAV delivery vehicle to facilitate improved regulation of the particular pro-opiomelanocortin polypeptide's activity, solubility, stability, expression, or efficacy in vitro and/or in vivo.

The techniques of site-specific mutagenesis are well known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and. vector-mediated propagation that result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing. Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCRTM amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Qβ Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[a-thio]triphosphates in one strand of a restriction site (Walker et al., 1992), may also be useful in the amplification of nucleic acids in the present invention.

Strand Displacement Amplification (SDA) is another method of carrying out isothermal amplification of nucleic acids that involves multiple rounds of strand displacement and synthesis, i.e., nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods (including, but not limited to, those described in Great Britain Pat. Appl. No. 2 202 328, and PCT Intl. Pat. Appl. Publ. No. PCT/US89/01025; each of which is incorporated herein by reference in its entirety), may also be used in the practice of the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e., new templates are not produced from the resultant RNA transcripts. Other amplification methods include “RACE” (Frohman et al., 1990), and “one-sided PCR” (Ohara et al., 1989) which are well-known to those of skill in the art.

Methods based on ligation of two (or more) oligonucleotides in the presence of nucleic acid having the sequence of the resulting “di-oligonucleotide”, thereby amplifying the di-oligonucleotide (Wu and Dean, 1996, incorporated herein by reference in its entirety), may also be used in the amplification of DNA sequences of the present invention.

### 4.17 RIBOZYMES AND CATALYTIC RNA MOLECULES

In certain aspects of the invention, it may be desirable to employ one or more catalytic RNA molecules (ribozymes) to effect a reduction or elimination of expression of one or more native DNA or mRNA molecules, so as to prevent or reduce the amount of the translation product of such mRNAs. The use of ribozymes and their introduction into mammalian cells utilizing rAAV vectors have been widely described in both the scientific and patent literature. Ribozymes are biological catalysts consisting of only RNA. They promote a variety of reactions involving RNA and DNA molecules including site-specific cleavage, ligation, polymerization, and phosphoryl exchange (Cech, 1989; Cech, 1990). Ribozymes fall into three broad classes: (1) RNAse P, (2) self-splicing introns, and (3) self-cleaving viral agents. Self-cleaving agents include hepatitis delta virus and components of plant virus satellite RNAs that sever the RNA genome as part of a rolling-circle mode of replication. Because of their small size and great specificity, ribozymes have the greatest potential for biotechnical applications. The ability of ribozymes to cleave other RNA molecules at specific sites in a catalytic manner has brought them into consideration as inhibitors of viral replication or of cell proliferation and gives them potential advantage over antisense RNA. Indeed, ribozymes have already been used to cleave viral targets and oncogene products in living cells (Koizumi et al., 1992; Kashani-Sabet et al., 1992; Taylor and Rossi, 1991; von-Weizsacker et al., 1992; Ojwang et al., 1992; Stephenson and Gibson, 1991; Yu et al., 1993; Xing and Whitton, 1993; Little and Lee, 1995).

Two kinds of ribozymes have been employed widely, hairpins and hammerheads. Both catalyze sequence-specific cleavage resulting in products with a 5N hydroxyl and a 2N,3N-cyclic phosphate. Hammerhead ribozymes have been used more commonly, because they impose few restrictions on the target site. Hairpin ribozymes are more stable and, consequently, function better than hammerheads at physiologic temperature and magnesium concentrations.

A number of patents have issued describing various ribozymes and methods for designing ribozymes. See, for example, U.S. Pat. Nos. 5,646,031; 5,646,020; 5,639,655; 5,093,246; 4,987,071; 5,116,742; and 5,037,746, each specifically incorporated herein by reference in its entirety. However, the ability of ribozymes to provide therapeutic benefit in vivo has not yet been demonstrated.

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 (specifically incorporated herein by reference) reports that certain ribozymes can act as endonucleases with a sequence-specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Six basic varieties of naturally occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., 1992). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al. (1990) and U.S. Pat. No. 5,631,359 (specifically incorporated herein by reference). An example of the hepatitis δ virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in U.S. Pat. No. 4,987,071 (specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

In the practice of the invention, it may be important to produce enzymatic cleaving agents that exhibit a high degree of specificity for the RNA of a specific intracellular target, such as, for example, a sequence that encodes a protein or other biological molecule that could alter, reduce, or inactivate the pro-opiomelanocortin constructs described herein. In such cases, it may be desirable to decrease, or prevent the expression of one or more biologically-active molecules in the cell which would interfere with the activity or finction of the AAV-vectored pro-opiomelanocortin constructs. Alternatively, it may be desirable to provide cells with a ribozyme construct that would inactive an aberrant native pro-opiomelanocortin gene, or another gene in the pathway(s) involved in pro-opiomelanocortin activity. Inactivation of such mutant genes could readily be achieved such that the exogenous supplied biologically-active pro-opiomelanocortin construct is not cleaved or inactivated by the ribozyme, but the native, or mutated pro-opiomelanocortin gene is cleaved, and thereby inactivated.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Scanlon et al., 1991; Kashani-Sabet et aL, 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and Int. Pat. Appl. Publ. No. WO 94/02595 (each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure, as described herein. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozymes of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically >98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-allyl, 2′-flouro, 2′-o-methyl, 2′-H (for a review see e.g., Usman and Cedergren, 1992). Ribozymes may be purified by gel electrophoresis using general methods or by high-pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al, 1990; Pieken et aL, 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

A preferred means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells (Elroy-Stein and Moss, 1990; Gao and Huang, 1993; Lieber et al., 1993; Zhou et al., 1990). Ribozymes expressed from such promoters can function in mammalian cells (Kashani-Sabet et al., 1992; Ojwang et aL, 1992; Chen et al., 1992; Yu et al., 1993; L'Huillier et al., 1992; Lisziewicz et aL, 1993). Although incorporation of the present ribozyme constructs into adeno-associated viral vectors is preferred, such transcription units can be incorporated into a variety of vectors for introduction into mammalian cells, including but not restricted to, plasmid DNA vectors, other viral DNA vectors (such as adenovirus vectors), or viral RNA vectors (such as retroviral, semliki forest virus, sindbis virus vectors).

Sullivan et al. (Int. Pat. Appl. Publ. No. WO 94/02595) describes general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraocular, retinal, subretinal, intraperitoneal, intracerebroventricular, intrathecal delivery, and/or direct injection to one or more tissues of the brain. More detailed descriptions of ribozyme and rAAV vector delivery and administration are provided in Int. Pat. Appl. Publ. No. WO 94/02595 and Int. Pat. Appl. Publ. No. WO 93/23569, each specifically incorporated herein by reference.

Ribozymes and the AAV vectored-constructs of the present invention may be used to inhibit gene expression and define the role (essentially) of specified gene products in the progression of one or more neural diseases, dysfunctions, cancers,, and/or disorders. In this manner, other genetic targets may be defined as important mediators of the disease. These studies lead to better treatment of the disease progression by affording the possibility of combination therapies (e.g., multiple ribozymes targeted to different genes, ribozymes coupled with known small molecule inhibitors, or intermittent treatment with combinations of ribozymes and/or other chemical or biological molecules).

### 4.18 ANTISENSE POLYNUCLEOTIDES AND OLIGONUCLEOTIDES

In certain embodiments, the AAV constructs of the invention will find utility in the delivery of antisense oligonucleotides and polynucleotides for inhibiting the expression of a selected mammalian mRNA in suitable host cells.

In the art the letters, A, G, C, T, and U respectively indicate nucleotides in which the nucleoside is Adenosine (Ade), Guanosine (Gua), Cytidine (Cyt), Thymidine (Thy), and Uridine (Ura). As used in the specification and claims, compounds that are “antisense” to a particular PNA, DNA or mRNA “sense” strand are nucleotide compounds that have a nucleoside sequence that is complementary to the sense strand. It will be understood by those skilled in the art that the present invention broadly includes polynucleotides and smaller oligonucleotide compounds that are capable of binding to the selected DNA or mRNA sense strand. It will also be understood that mRNA includes not only the ribonucleotide sequences encoding a protein, but also regions including the 5′-untranslated region, the 3′-untranslated region, the 5′-cap region and the intron/exon junction regions.

The invention includes compounds which are not strictly antisense; the compounds of the invention also include those polynucleotides and oligonucleotides that may have some bases that are not complementary to bases in the sense strand provided such compounds have sufficient binding affinity for the particular DNA or mRNA for which an inhibition of expression is desired. In addition, base modifications or the use of universal bases such as inosine in the oligonucleotides of the invention are contemplated within the scope of the subject invention.

The antisense compounds may have some or all of the phosphates in the nucleotides replaced by phosphorothioates (X=S) or methylphosphonates (X=CH₃) or other C₁₋₄ alkylphosphonates. The antisense compounds optionally may be further differentiated from native DNA by replacing one or both of the free hydroxy groups of the antisense molecule with C₁₋₄ alkoxy groups (R=C₁₋₄ alkoxy). As used herein, C₁₋₄ alkyl means a branched or unbranched hydrocarbon having 1 to 4 carbon-atoms.

The disclosed antisense compounds also may be substituted at the 3′ and/or 5′ ends by a substituted acridine derivative. As used herein, “substituted acridine,” means any acridine derivative capable of intercalating nucleotide strands such as DNA. Preferred substituted acridines are 2-methoxy-6-chloro-9-pentylaminoacridine, N-(6-chloro-2-methoxyacridinyl)-O-methoxydiisopropylaminophosphinyl-3-aminopropanol, and N-(6-chloro2-methoxyacridinyl)-O-methoxydiisopropylaminophosphinyl-5-aminopentanol. Other suitable acridine derivatives are readily apparent to persons skilled in the art. Additionally, as used herein “P(O)(O)-substituted acridine” means a phosphate covalently linked to a substitute acridine.

As used herein, the term “nucleotides” includes nucleotides in which the phosphate moiety is replaced by phosphorothioate or alkylphosphonate and the nucleotides may be substituted by substituted acridines.

In one embodiment, the antisense compounds of the invention differ from native DNA by the modification of the phosphodiester backbone to extend the life of the antisense molecule. For example, the phosphates can be replaced by phosphorothioates. The ends of the molecule may also be optimally substituted by an acridine derivative that intercalates nucleotide strands of DNA. Intl. Pat. Appl. Publ. No. WO 98/13526 and U.S. Pat. No. 5,849,902 (each specifically incorporated herein by reference in its entirety) describe a method of preparing three component chimeric antisense compositions, and discuss many of the currently available methodologies for synthesis of substituted oligonucleotides having improved antisense characteristics and/or half-life.

The reaction scheme involves ¹H-tetrazole-catalyzed coupling of phosphoramidites to give phosphate intermediates that are subsequently reacted with sulfur in 2,6-lutidine to generate phosphate compounds. Oligonucleotide compounds are prepared by treating the phosphate compounds with thiophenoxide (1:2:2 thiophenol/triethylamine/tetrahydrofuran, room temperature, 1 hr). The reaction sequence is repeated until an oligonucleotide compound of the desired length has been prepared. The compounds are cleaved from the support by treating with ammonium hydroxide at room temperature for 1 hr and then are further deprotected by heating at about 50° C. overnight to yield preferred antisense compounds.

Selection of antisense compositions specific for a given gene sequence is based upon analysis of the chosen target sequence and determination of secondary structure, Tₘ, binding energy, relative stability, and antisense compositions were selected based upon their relative inability to form dimers, hairpins, or other secondary structures that would reduce or prohibit specific binding to the target mRNA in a host cell. Highly preferred target regions of the mRNA, are those that are at or near the AUG translation initiation codon, and those sequences that were substantially complementary to 5′ regions of the mRNA. These secondary structure analyses and target site selection considerations were performed using v.4 of the OLIGO primer analysis software (Rychlik, 1997) and the BLASTN 2.0.5 algorithm software (Altschul et al., 1997).

### 4.19 BIOLOGICAL FUNCTIONAL EQUIVALENTS

Modification and changes may be made in the structure of the rAAV vector-delivered pro-opiomelanocortin compositions, or the polynucleotides and/or encoded polypeptides of the present invention and still obtain a functional molecule that encodes a pro-opiomelanocortin polypeptide with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 3.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 3

| | |
|---|---|
| AMINO ACIDS | CODONS |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

### 4.20 EXEMPLARY DEFINITIONS

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription. Exemplary regulatory elements include, but are not limited to, enhancers, post-transcriptional elements, transcriptional control sequences, and such like.

Structural gene: A polynucleotide, such as a gene, that is expressed to produce an encoded peptide, polypeptide, protein, ribozyme, catalytic RNA molecule, or antisense molecule.

Transformation: A process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), MRNA stability controlling sequence(s), etc.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, 2, 3, 4, 5, 6, 7, 8, 9, or 10 base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the MRNA and will therefore be highly efficient in reducing, and/or even inhibiting the translation of the target mRNA sequence into polypeptide product.

Substantially complementary oligonucleotide sequences will be greater than about 80 percent complementary (or ‘% exact-match’) to the corresponding mRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding MRNA target sequence to which the oligonucleotide specifically binds, and may in certain embodiments be greater than about 95 percent complementary to the corresponding MRNA target sequence to which the oligonucleotide specifically binds, and even up to and including 96%, 97%, 98%, 99%, and even 100% exact match complementary to all or a portion of the target mRNA to which the designed oligonucleotide specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

### 5.0 EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

### Central Pro-Opiomelanocortin Gene Delivery Results in Hypophagia, Reduced Visceral Adiposity and Improved Insulin Sensitivity in Genetically Obese Zucker Rats

An rAAV-based plasmid encoding POMC was constructed and packaged into rAAV-POMC. 11-week-old male obese Zucker rats were administered either the rAAV-POMC or control vector (n=6 for each group) by bilateral injections (1.3E9 particle/injection in 3 ml) into the hypothalamic arcuate nucleus. Daily food intake and body weight were monitored for 38 days. Hypothalamic POMC and AgRP expression levels were evaluated by relative quantitative RT-PCR using QuantumRNA 18S Internal Standards kit (Ambion). Melanocortin signaling was assessed by phosphorylation of CREB (P-CREB) in the hypothalamus. Fasting serum leptin and insulin were measured by RIA, and total cholesterol and glucose levels were determined by enzymatic colorimetric kits. Induction of UCP1 protein in brown adipose tissue (BAT) was assessed by Western analysis as one measure of thermogenesis.

Table 4 shows uncoupling protein 1 immunoreactivity and BAT parameters at day 38 post rAAV-POMC or rAAV-Control delivery.

TABLE 4

| | Treatment | |
|---|---|---|
| | rAAV-Control | rAAV-POMC |
| BAT weight (mg) | 2193 ± 275 | 1366 ± 189* |
| BAT protein (mg/g BAT) | 23.4 ± 1.4 | 27.2 ± 1.8 |
| BAT protein (mg/total BAT) | 50.2 ± 5.2 | 38.3 ± 7.2 |
| UCP1 protein (arbitrary units/g BAT) | 100 ± 17 | 662 ± 114** |
| UCP1 protein (arbitrary units/ | 100 ± 33 | 412 ± 104* |
| total BAT) | | |

For UCP1 protein, the levels in rAAV-Control rats are set to 100 and SE adjusted proportionally.
*P < 0.05 and **P < 0.01 vs. control.

Following rAAV-mediated POMC gene delivery: (1) There was 4-fold increase in POMC expression in the hypothalamus of obese Zucker rats at day 38 suggesting sustained POMC overexpression; (2) Hypothalamic melanocortin signaling indicated by P-CREB increased by 62%; (3) Hypothalamic AgRP MRNA levels were unchanged; (4) The anorexic response was sustained throughout the 38-day experimental period. (5) Body weight gain and visceral adiposity were reduced. (6) Fasting serum leptin, insulin and cholesterol levels were significantly reduced; (7) BAT UCP1 synthesis was enhanced by over 4-fold.

In conclusion, central POMC gene delivery circumvents inherent leptin resistance, reducing weight gain and visceral adiposity in obese Zucker rats. POMC gene delivery appears to improve glucose and cholesterol metabolism and insulin sensitivity. The sustained hypophagia and augmentation of BAT thermogenesis are the likely mechanisms underlying these improvements. Prolonged suppression in food consumption by POMC gene delivery, as compared to the transient response following MC agonists, may present a promising strategy for long-term weight maintenance.

### 5.2 Example 2

### Activation of Central Melanocortin Pathway Bypass Deflective Leptin Signaling

Zucker (fa/fa) rats with defective leptin receptors are obese, hyperphagic and hyperinsulinemic. For testing whether chronic activation of the central melanocortin pathway can bypass the defective leptin signaling and normalize altered energy homeostasis in these rats, recombinant adeno-associated virus encoding pro-opiomelanocortin (rAAV-POMC) or control vector was delivered bilaterally into the basal hypothalamus with coordinates targeting the arcuate nucleus. Thirty-eight days after POMC gene delivery, hypothalamic POMC expression increased 4-fold and melanocortin signaling (indicated by phosphorylation of CREB) increased by 62% with respect to controls. There was a sustained reduction in food intake, a moderate but significant attenuation of weight gain and a 24% decrease in visceral adiposity in rAAV-POMC rats. POMC gene delivery enhanced uncoupling protein 1 in brown adipose tissue (BAT) by more than 4-fold. Fasting serum leptin, insulin and cholesterol levels were also significantly reduced by rAAV-POMC treatment. This study demonstrates that targeted POMC gene delivery in the hypothalamus suppresses food intake and weight gain and reduces visceral adiposity and hyperinsulinemia in leptin-resistant obese Zucker rats. The mechanisms involve the sustained hypophagia and the augmentation of thermogenesis in BAT.

Using a leptin- and insulin-resistant rodent model, the inventors examined whether overproduction of POMC in the hypothalamus would reduce body mass and adiposity and improve glucose metabolism in obese Zucker rats. In addition, some evidence suggests that tachyphylaxis to the MC-mediated reduction in food intake develops after chronic pharmacological treatment of MC agonists in rodents (McMinn et al., 2000; Pierroz et al., 2002). Thus, the second aspect of this example is a study of the consequences of long-term targeted overexpression of POMC on the MC-mediated anorexic response.

Recent successes in using recombinant adeno-associated virus (rAAV) to obtain long-term expression of transgenes provide an opportunity to test these hypotheses (Monahan and Samulski, 2000). There are many advantages of using rAAV, including nonpathogenicity, nonimmunogenicity, high viability of the virion, and most important, long-term expression of the delivered transgene. The rAAV type 2 vector has been uniquely successful as gene transfer vector into the CNS (Xu et al., 2001).

In the present example, an rAAV type 2 vector encoding murine POMC (rAAV-POMC) was used to assess the long-term consequences of POMC gene delivery on energy balance, BAT thermogenesis and hypothalamic MC signal transduction in obese Zucker rats. To this end, rAAV-POMC or control vectors were administered bilaterally into the arcuate nucleus of the hypothalamus of obese Zucker rats for 38 days, and food intake, body weight, adiposity, serum hormone and metabolite levels, BAT UCP1 protein, hypothalamic POMC, AgRP mRNA levels, and hypothalamic phosphorylation of the cAMP response element binding protein (CREB) were assessed (Montminy et al., 1990).

### 5.2.1 RESEARCH AND METHODS

### 5.2.1.1 Construction of RAAV Vector Plasmids

pTR-CBA-POMC encodes murine POMC cDNA (a gift from Dr. James Roberts) (Uhler and Herbert, 1983) under the control of the hybrid cytomegalovirus (CMV) immediate early enhancer/chicken β-actin (CBA) promoter (Daly et al., 2001). The woodchuck hepatitis virus post-transcriptional regulatory element (WPRE) is placed downstream of the POMC transgene to enhance its expression (Loeb et al., 1999). The control plasmid, termed pTR-control, is similar to pTR-CBA-POMC except for the incorporation of the cDNA encoding an enhanced form of green fluorescent protein instead of POMC cDNA. The control vector was described previously (Li et al., 2002).

### 5.2.1.2 In Vitro Analysis of PTR-CBA-POMC Plasmid

The pTR-CBA-POMC construct was tested for in vitro expression of POMC by transfecting HEK 293 cells using FuGENE 6 transfection reagent (Roche Diagnostics, Indianapolis, Ind.) according to the protocol provided by the manufacturer. One day after the transfection, cells were harvested and total RNA was isolated. POMC mRNA levels were analyzed by RT-PCR.

### 5.2.1.3 Packaging of RAAV Vectors

Vectors were packaged, purified, concentrated, and titered as previously described (Zolotukhin et al., 1999). The titers for both rAAV-POMC and rAAV-control vectors used in this study were 4.26×10¹¹ physical particles/ml, and the ratios of physical-to-infectious particles for both vectors were less than 30.

### 5.2.1.4 Animals

Nine-week-old male obese Zucker (fa/fa) rats were obtained from Charles River (Wilmington, Mass.). Upon arrival, rats were examined and remained in quarantine for two weeks. Animals were cared for in accordance with the principles of the NIH Guide to the Care and Use of Experimental Animals. Rats were housed individually with a 12:12 hr light/dark cycle (0700 to 1900 hr). Standard Purina 5001 rodent diet and water were provided ad libitum.

### 5.2.1.5 RAAV Vector Administration

Under 6 mg/kg xylazine (Phoenix Pharmaceutical, St. Joseph, Mo.) and 60 mg/kg ketamine (Monarch Pharmaceuticals, Bristol, Tenn.) anesthesia, rats were administered bilaterally (1.28 x 109 particles/injection in 3 μl) rAAV-POMC (n=6) or rAAV-control (n=6) into the basal hypothalamus with coordinates targeting the arcuate nucleus. The coordinates for injection into the hypothalamus were 3.14 mm posterior to bregma, ±0.4 mm lateral to the midsaggital suture, and 10 mm ventral from the skull surface. On each side, a small hole was drilled through the skull and a 23-gauge stainless steel cannula was inserted followed by an injection cannula. With the use of a 10-μl Hamilton syringe, a 3-μl volume of virus stocks was delivered over 5 min to each site. The needles remained in place at the injection site for an additional 5 min. At the time of surgery, rats were injected with the analgesic Buprenex (0.05 mg/kg; Reckitt and Colman, Richmond, VA).

### 5.2.1.6 Tissue Harvesting and Preparation

Rats were fasted overnight and sacrificed by cervical dislocation under 85 mg/kg pentobarbital anesthesia. Blood samples were collected by heart puncture, and serum was harvested by a 15-min centrifugation in serum separator tubes. The circulatory system was perfused with 30 ml of cold saline, BAT, perirenal white adipose tissue (PWAT), and retroperitoneal white adipose tissues (RTWAT) were excised. The hypothalamus was removed by making an incision medial to piriform lobes, caudal to the optic chiasm and anterior to the cerebral crus to a depth of 2-3 mm. Tissues were stored at −80° C. until analysis. For Western analysis, hypothalamus and BAT were homogenized in 0.3 ml of 10 mmol/l Tris-HCl (pH 6.8), 2% SDS, and 0.08 μg/ml okadaic acid. Protease inhibitors, 1 mmol/l phenylmethylsulfonyl fluoride (PMSF), 0.1 mmol/l benzamidine, and 2 μmol/l leupeptin were also present. Homogenates were immediately boiled for 2 min, cooled on ice, and stored frozen at −80° C. Protein was determined using the DC protein assay kit (Bio-Rad, Hercules, Calif.). BAT samples were filtered through a 0.45-μm syringe filter (Whatman, Clifton, N.J.) to remove lipid particles before protein measurements.

### 5.2.1.7 Serum Leptin, Insulin, Glucose, Free Fatty Acids, and Cholecterol

Serum leptin and insulin were measured using rat radioimmunoassay kits (Linco Research, St. Charles, Mo.). Serum free fatty acids (FFAs) and total cholesterol were determined by enzymatic colorimetric kits from WAKO Chemicals (Neuss, Germany). Serum glucose was via a colorimetric reaction with Trinder, the Sigma Diagnostics Glucose reagent (St. Louis, Mo.).

### 5.2.1.8 Reverse Transcriptase-Polymerase Chain Reaction (RT-PCR™)

POMC and AgRP expression in the hypothalamus were identified by relative quantitative RT-PCR™ using QuantumRNA 18S Internal Standards kit (Ambion, Austin, Tex.). Total cellular RNA was extracted as described (Li et al., 2002) and treated with RNase-free DNase (Ambion). First-strand cDNA synthesis was generated from 2 μg of RNA in a 20-μl volume using random primers (Life Technologies, Rockville, Md.) containing 200 units of M-MLV reverse transcriptase (Life Technologies). Relative quantitative PCR was performed by multiplexing corresponding primers (POMC sense 5′-GCTTGCAAACTCGACCTCTC-3′, (SEQ ID NO:41) antisense 5′-CTTGATGATGGCGTTCTTGA-3′ (SEQ ID NO:42); AgRP sense 5′-AGGGCATCAGAAGGCCTGACCA-3′ (SEQ ID NO:43), antisense 5′-CTTGAAGAAGCGGCAGTAGCAC-3′) (SEQ ID NO:44), 18S primers, and competimers and co-amplifying. Linearity for the POMC and AgRP amplicons was determined to be between 20 and 29 cycles. The optimum ratio of 18S primer to competimer was 1:4 for POMC and 1:9 for AgRP. PCR was performed at 94° C. denaturation for 60 sec, 59° C. annealing temperature for 45 sec, and 72° C. elongation temperature for 60 sec for 26 (POMC) or 24 (AgRP) cycles. The PCR product was electrophoresed on acrylamide gel and stained with SYBR green (Molecular Probes, Eugene, Oreg.). Gels were scanned using a STORM fluorescent scanner, and data were analyzed using InageQuant (Molecular Dynamics). The relative values of POMC and AgRP mRNA were derived from dividing the signal obtained for corresponding amplicon by that for 18S amplicon.

### 5.2.1.9 Creb and Phosphorlylated CREB (P-CREB) Assay

Immunoreactive CREB and P-CREB were determined with a PhosphoPlus CREB (Ser 133) antibody kit (New England Biolabs, Beverly, Mass.). Hypothalamic samples (40 μg) prepared as described above were separated on an SDS-PAGE gel and electrotransferred to nitrocellulose membrane. Immunoreactivity was assessed on separate membranes with antibodies specific to CREB (phosphorylated and unphosphorylated) and antibodies specific to Serinel33-phosphorylated CREB. Immunoreactivity was visualized by enhanced chemiluminescent detection (Amersham Pharmacia Biotech) and quantified by video densitometry (Bio-Rad).

### 5.2.1.10 UCP1 Protein

Immunoreactive UCP1 in BAT homogenates (20 μg) was determined as described for CREB, except that an antibody specific to rat UCP1 (Linco Research) was used.

### 5.2.1.11 Statistical Analysis

Results are presented as mean ±SE. Repeated-measure ANOVA was used for analyses of body weight and food intake. When the main effect was significant, a post-hoc t-test was applied to determine individual differences between means. For all other data, unpaired two-tailed Student's t-test was used. A value of P<0.05 was considered significant.

### 5.2.2 RESULTS

### 5.2.2.1 Transient Expression of POMC IN HEK 293 Cells

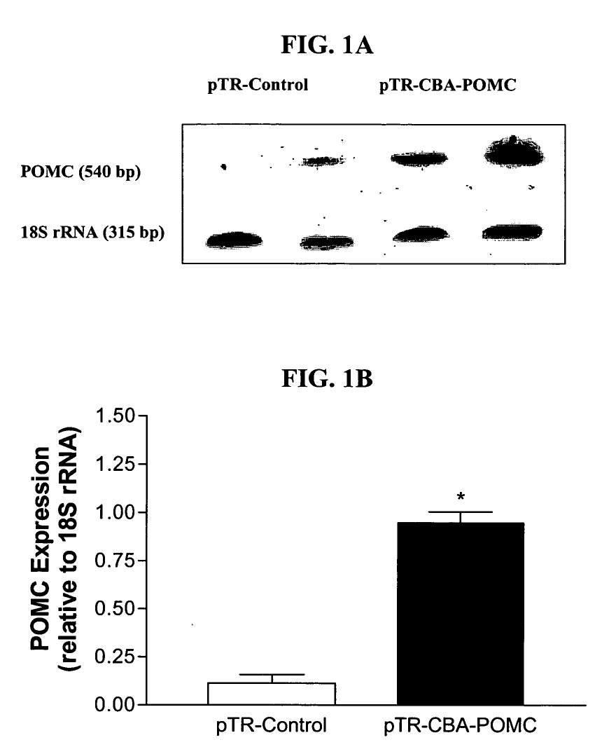

HEK 293 cells were transfected with pTR-CBA-POMC or pTR-control plasmids (FIG. 1A and FIG. 1B). Twenty-four hours after the transfection, total POMC MRNA expression was measured by relative quantitative RT-PCR™. The primers used do not discriminate among mouse, rat and human POMC; thus, the measured POMC mRNA represents both transgene mouse POMC and endogenous HEK 293 human POMC mRNA. The pTR-CBA-POMC transfection increased POMC expression in HEK 293 cells by more than eight-fold as compared to pTR-control (P<0.001).

### 5.2.2.2 POMC Expression in the Hypothalamus of Obese Zucker Rats

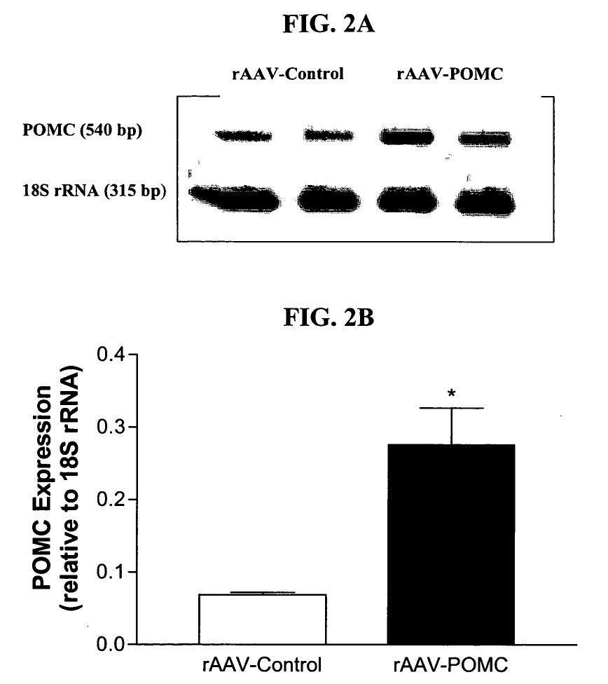

To verify the overexpression of the POMC transgene after central viral delivery, POMC mRNA was measured in the hypothalamus by RT-PCRTM (FIG. 2A and FIG. 2B). Thirty eight days after vector delivery, hypothalamic POMC mRNA levels were elevated by four-fold in obese Zucker rats that were given rAAV-POMC as compared with those that were given rAAV-control (P<0.02). The ratio of hypothalamic POMC to 18S rRNA expression ranged from 0.061 to 0.073 for control animals and form 0.186 to 0.435 for rAAV-POMC-treated rats. Therefore, the POMC mRNA levels were at least two-fold higher in all of the rAAV-POMC rats as compared with the mean levels in the control animals.

### 5.2.2.3 Melanocortin Signal Transduction and AGRP MRNA Levels

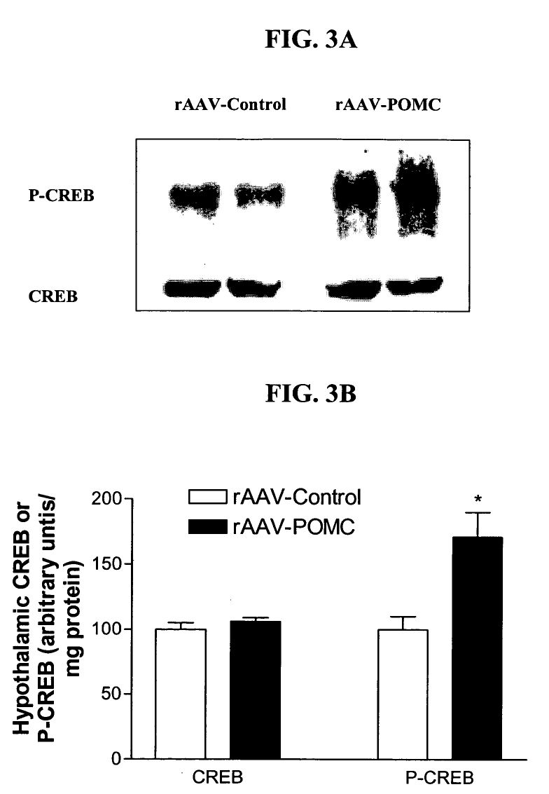

α-MSH, one of the cleavage products of POMC, is reported to exert its anorexic and thermogenic effects via activation of hypothalamic melanocortin receptors and subsequent phosphorylation of the transcription factor CREB (P-CREB) (Sarkar et al., 2002). Hypothalamic CREB and P-CREB immunoreactivities were assessed 38 days after POMC vector delivery. Phosphorylation of CREB was elevated by 62% in the hypothalami of rats with rAAV-POMC treatment, whereas total CREB levels were unchanged (FIG. 3A and FIG. 3B).

The expression of AgRP, an endogenous antagonist of MC receptors, is presumably regulated by leptin in normal animals (Schwartz et al., 1996; Schwartz et al., 1997; Cheung et al., 1997; Baskin et al., 1999; Elias et al., 2000). RT-PCR™ revealed that hypothalamic AgRP expression levels were unchanged in rats that were given rAVV-POMC as compared with control rats at sacrifice (0.927±0.092 vs. 0.995±0.053 relative to 18S rRNA; P>0.5).

### 5.2.2.4 Body Weight and Food Intake

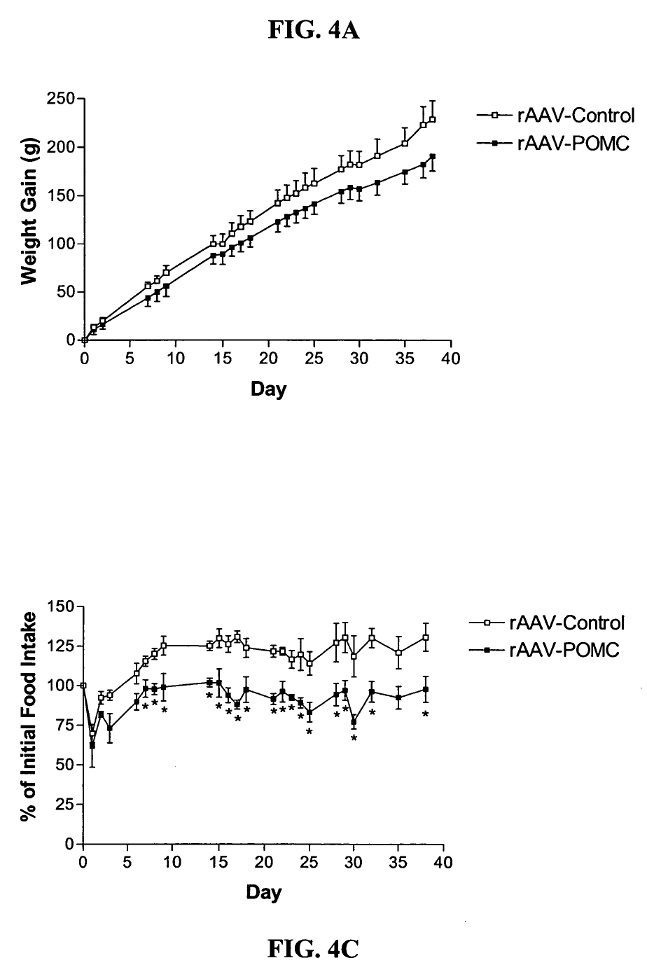

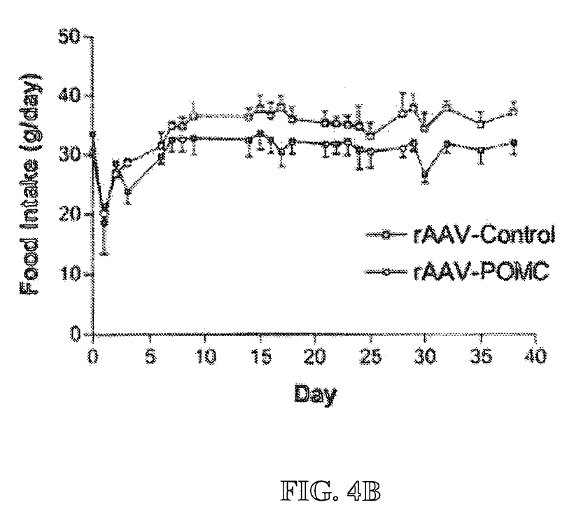

Bilateral delivery of rAAV-POMC into the basal hypothalamus reduced weight gain and food intake in obese Zucker rats (FIG. 4A, FIG. 4B and FIG. 4C). Before and on the day of vector delivery, average body weight of rAAV-POMC-treated rats was comparable to that of control rats (428±18 vs. 403±23 g at day 0). After vector delivery, the rats that were given rAAV-POMC consistently gained less weight, and the difference in body mass between the two groups gradually increased over the thirty-eight days (FIG. 4A). Because daily food consumption of obese Zucker rats varied noticeably between individual rats (FIG. 4B), daily food intake after vector delivery was also expressed as percentage of individual baseline levels, represented by the average daily food intake one week before vector administration (FIG. 4C). Hypothalamic delivery of rAAV-POMC induced a sustained anorexic response in these obese rats. The inhibition of food intake became statistically significant starting at day 7 after POMC vector delivery and lasted for the duration of the experiment (FIG. 4B and FIG. 4C).

### 5.2.2.5 Visceral Adiposity and Serum Leptin Levels

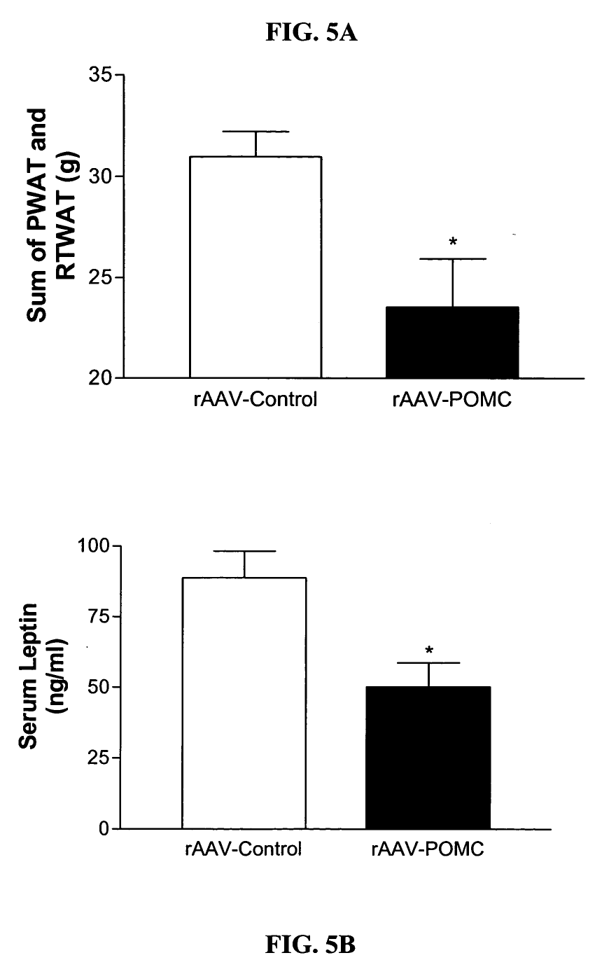

Because POMC gene therapy reduced the weight gain of the obese Zucker rats, body adiposity levels were assessed. Thirty-eight days after central POMC gene delivery, there were significant reductions in the visceral adiposity, as reflected by a 24% reduction in the sum of the PWAT and RTWAT (P<0.05) in rAAV-POMC-treated compared with control rats (FIG. 5A). Fasting serum leptin levels, known to be highly correlated with body fat mass (Frederich et al., 1995), were 43.5% lower in the rAAV-POMC group compared with the control group (FIG. 5B).

### 5.2.2.6 Fasting Serum Insulin, Glucoes FFAS, and Cholesterol

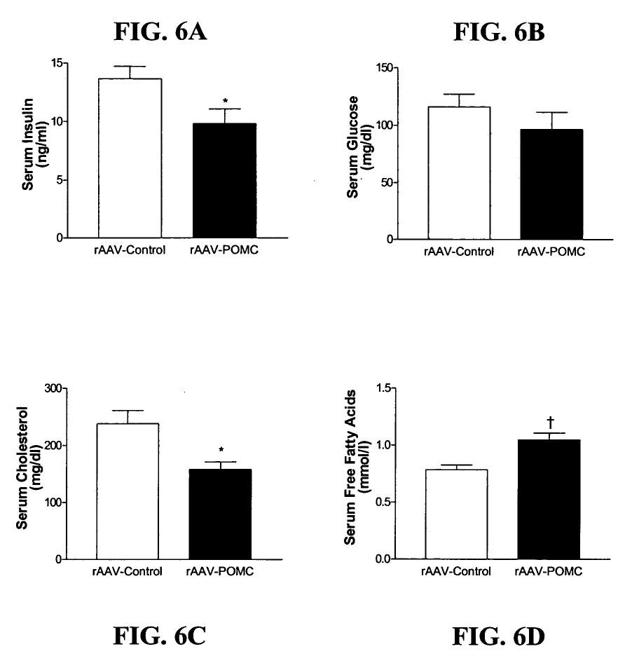

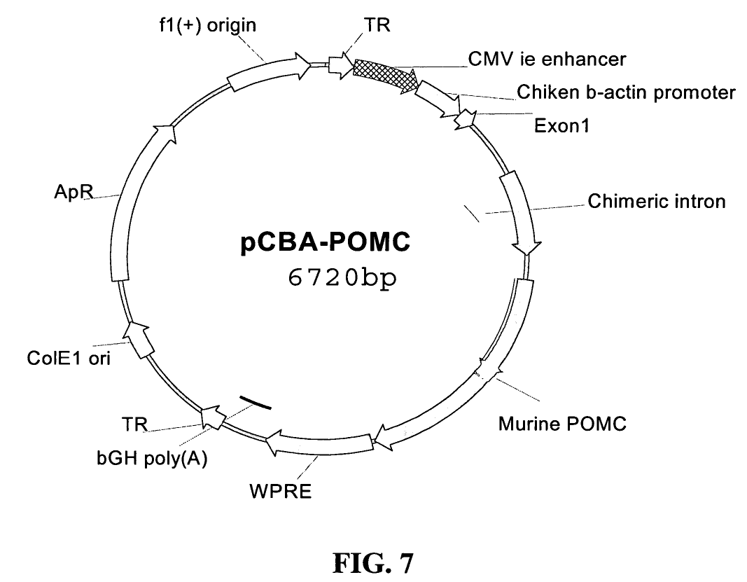

At sacrifice, serum insulin was significantly reduced by rAAV-POMC as compared with rAAV-control, and serum glucose tended toward a decrease (FIG. 6A and FIG. 6B). The rAAV-POMC delivery also reduced serum total cholesterol levels by 34.5% and increased FFA levels by 33% compared with rAAV-control (FIG. 6C and FIG. 6D).

### 5.2.2.7 Brown Adipose Tisseu (Bat)

Induction of UCP 1 in BAT is an important marker for enhanced thermogenesis and, thus, energy expenditure in rodents (Scarpace et al., 1997). In the present study, the UCP1 protein levels were examined 38 days after POMC gene delivery. Total BAT weight markedly declined with rAAV-POMC treatment, whereas the protein concentration (per unit of BAT) increased slightly, suggesting that the reduction in BAT mass was due to the lipolysis associated with the activation of BAT. This was further supported by a 4-fold increase in BAT UCP1 protein levels in the rAAV-POMC-treated compared with control rats (Table 4).

### 5.2.3 Discussion

The present example describes the long-term consequences of central rAAV-POMC gene therapy in obese Zucker rats with inherent defective leptin receptors and deficient endogenous POMC expression. These findings are in agreement with several previous pharmacological studies indicating that direct activation of the central MC system is effective in partially reversing the hyperphagia and obesity in obese Zucker rats (Hwa et al., 2001; Cettour-Rose and Rohner-Jeanrenaud, 2002). The present gene therapy approach, in particular, resulted in long-term overexpression of POMC in the basal hypothalamus, and these obese Zucker rats responded with significant reductions in both food intake and weight gain. The level of hypothalamic P-CREB, the putative active form of the transcriptional factor mediating MC3/MC4 receptor signaling (Montminy et al., 1990; Sarkar et al., 2002), was significantly elevated in the obese Zucker rats, whereas hypothalamic AgRP mRNA was unaffected at day 38 after POMC vector delivery. Thus, it seems that long-term POMC gene therapy attenuates weight gain primarily through activation of the central MC system characterized by increased hypothalamic POMC MRNA levels, CREB phosphorylation, and unchanged AgRP expression. Because POMC vector delivery was aimed at the arcuate nucleus, where POMC-expressing neurons are located, it is most likely that POMC overexpression in these neurons is mainly responsible for the observed physiologic consequences. However, given the proximity of the third ventricle to the arcuate nucleus, the possibility exists that some of the viral vectors entered the third ventricle. The vectors could also diffuse out of the arcuate nucleus, resulting in the transduction of neurons outside the arcuate nucleus. The potential ectopic expression of POMC in the brain might account for some of the responses observed. In such cases, the responses would be expected to be more pharmacologic rather than physiologic in nature.

The present example provides several distinct sets of salient findings. First, these data suggest that an increase in energy expenditure contributes to the reduced weight gain and visceral adiposity after POMC gene delivery. It is known that pharmacologic activation of the MC system augments energy expenditure in rodents. For example, normal animals treated with the a-MSH analog MTII have elevated levels of BAT UCP1 expression compared with pair-fed controls (Cettour-Rose and Rohner-Jeanrenaud, 2002). The unique UCPl-mediated nonshivering thermogenesis in BAT represents an essential element in adaptive energy expenditure in rodents. An increase in UCP1 protein is indicative of increased BAT-facilitated energy expenditure. Obese Zucker rats, however, have impaired BAT thermogenesis because of the genetically defective leptin receptors (Levin et al., 1984). Despite this inherent problem, a four-fold increase in BAT UCP1 was observed at 38 days after POMC vector delivery in the obese Zucker rats, indicating markedly stimulated BAT thermogenesis. Although lacking direct measurement of whole body energy expenditure, it is speculated that, in addition to the hypophagia, an increase in energy expenditure plays a part in mediating the fat- and weight-trimming effects of central POMC gene therapy.

Second, rAAV-mediated POMC gene delivery produces an impressive reduction in adiposity. For example, by the end of the 38-day POMC gene therapy, PWAT and RTWAT combined were decreased by 24% when compared with controls. In addition, a key indicator of whole body mass (Frederich et al., 1995), fasting serum leptin levels, was also reduced by 44%. Central chronic infusion of α-MSH has been shown by one study to preferentially reduce visceral fat mass as opposed to lean body mass in lean rats (Obici et al., 2001). If such a preference in action is proven to exist, strategies based on MC activation to treat obesity will offer an apparent advantage over some other weight control remedies that indiscriminately reduce both lean and fat body mass through suppression in food consumption.

Third, rAAV-POMC gene delivery seems to improve glucose metabolism. The obese Zucker rats are insulin-resistant as indicated by remarkably high levels of serum insulin. Central POMC gene therapy decreased fasting serum insulin, and it also generated a downward trend in serum glucose levels. These data suggest improved glucose metabolism and insulin sensitivity by POMC gene delivery. This is consistent with previous findings that central MC receptor activation reduces insulin release from the pancreas and enhances glucose metabolism (Fan et al., 1997; Obici et al., 2001). However, without a pair-fed group to control for the effect of food intake, it is not certain whether the improvement in glucose metabolism is directly related to the increased central POMC expression or a consequence of the decreased food consumption and body weight. In addition to its impact on insulin and glucose, POMC gene delivery lowered total serum cholesterol levels in obese Zucker rats. Thus, central activation of the MC system has a cholesterol-reducing effect in these obese animals that possess both leptin and insulin resistance. The mechanism underlying the reduction in cholesterol is currently unknown, and one explanation could be that insulin-mediated stimulation of cholesterol synthesis is impeded after a fall in circulating insulin levels (Horton et al., 2002).

The most surprising finding from this example is that the anorexic response to rAAV-POMC was sustained throughout the 38-day experimental period. This is in sharp contrast to all previous studies in which there was a rapid attenuation of the anorexic response after pharmacologic infusions of MCs in both normal rats and rats with diet-induced obesity and in mice (McMinn et al., 2000; Pierroz et al., 2002). The suppression in food intake lasted no longer than 4 days in any of these studies. In the case of obese Zucker rats, one study noted a 3-day anorexic response to chronic pharmacologic administration of MTII (Cettour-Rose and Rohner-Jeanrenaud, 2002). Therefore, the sustained anorexic response observed over 38 days may be unique to central POMC gene delivery. With this procedure, the presumed overproduction of α-MSH is derived from POMC with assistance from a variety of endogenous enzymes such as prehormone convertases, carboxypeptidase E, and peptidyl α-amidating mono-oxygenase (Pritchard et al., 2002). This endogenously regulated production of α-MSH may help to prevent the rapid desensitization witnessed in previous pharmacological studies. In addition, obese Zucker rats, in comparison with their lean counterparts, are associated with reduced POMC expression in the arcuate nucleus, lower amount of α-MSH peptide in the paraventricular nucleus, and higher MC4 receptor densities in several hypothalamic regions pivotal to energy regulation (Kim et al., 2000; Harrold et al., 1999). These factors may also contribute to the prolonged responsiveness to POMC gene delivery in obese Zucker rats. Central MC signaling has been implicated in the development of cachexia (Marks et al., 2003), and the possibility that the sustained anorexic response by central POMC gene delivery might be in part a mimic of inflammatory-like activities of POMC products cannot be ruled out, although in the present study, POMC gene delivery seemed to reduce visceral adiposity to a greater extent as compared with the decrease in whole body mass, an observation inconsistent with cachexigenic action.

In summary, the present study demonstrates that targeted POMC gene delivery to the hypothalamus suppresses food intake and weight gain and reduces visceral adiposity in genetically obese Zucker rats. This treatment also appears to improve glucose and cholesterol metabolism and insulin sensitivity. The sustained hypophagia and augmentation of thermogenesis in BAT are the likely mechanisms underlying these improvements.

### 5.3 Example 3

### Hypothalamic Pro-Opiomelanocortin Gene Delivery Ameliorates Obesity and Glucose intolerance in Aged Rats

Melanocortins (MCs) are bioactive peptides derived from a common pre-hormone, pro-opiomelanocortin (POMC), and the central melanocortin system plays a critical role in the regulation of energy balance and glucose metabolism (Cone, 1999; Fan et al., 1997; Huszar et al., 1997; Mizuno and Mobbs, 1999; Butler et al., 2000; Obici et al., 2001). Reduced expression of hypothalamic POMC is associated with obesity syndromes caused by mutations in any of several genes, including leptin receptor (Mizuno et al., 1998; Kim et al., 2000), tubby (Guan et al., 1998), or Nh1h2 (Good et al., 1997); by hypothalamic damage (Bergen et al., 1998); and, perhaps most commonly, by aging (Mobbs et al., 2001). Reduced hypothalamic POMC mRNA may be one contributor to the obese phenotypes in these models because mutations in the POMC gene cause obesity in mice (Yaswen et al., 1999) and humans (Krude et al., 1998). However, it is still unclear if normalization of central POMC tone can reverse obese phenotypes.

A common form of obesity, age-related obesity, is characterized by a progressive increase in body weight and visceral adiposity. This increase in body weight with age is a major risk factor for insulin resistance, diabetes and atherosclerotic cardiovascular disease (Gabriely et al., 2002). Aged mice and rats are associated with reduced hypothalamic POMC expression (Mobbs et al., 2001), and the induction of POMC by exogenous leptin is impaired with aging (Scarpace et al., 2002a). The male F1 hybrid of Fischer 344-Brown Norway rats (F344/BN) is a useful model for age-related obesity because these rats have a relatively long life span and gain a substantial amount of body weight and fat mass with aging (Li et al., 1997). F344/BN rats also display age-associated impairments in glucose metabolism and insulin responsiveness (Larkin et al., 2001). Although chronic pharmacological treatment of MC agonists in rodents, including aged rats, reduces food intake and increases energy expenditure, its effectiveness is limited by the rapid tachyphylaxis of the MC responses (Zhang et al., 2004; McMinn et al., 2000; Pierroz et al., 2002).

The advantages of using rAAV to obtain long-term transgene expression include site-specific integration within a defined region of human chromosome 19, the ability to efficiently transduce post-mitotic tissues, and a lack of pathogenicity and immunogenicity (Monahan and Samulski, 2000). Example 5.2 shows that a serotype 2 rAAV vector encoding POMC reduces visceral adiposity and improves insulin sensitivity in genetically obese Zucker rats (Li et al., 2003). Recently, a new serotype rAAV, rAAV type 5 has been developed and shown to be more efficient at transducing select tissues in vivo (Hildinger et al., 2001; Zabner et al., 2000). Therefore, in this example, a serotype 5 rAAV vector encoding murine POMC (rAAV-POMC) was used to assess the long-term consequences of POMC gene delivery on energy balance, glucose metabolism, BAT thermogenesis, and mRNA levels of hypothalamic neuropeptides and MC receptors in aged obese F344/BN rats. The results demonstrated that targeted POMC gene delivery in the hypothalamus reduces body weight and visceral adiposity, and improves glucose and fat metabolism in aged obese rats. The anorexia and stimulated BAT thermogenesis may, in part, underscore these effects.

### 5.3.1 MATERIALS AND METHODS

### 5.3.1.1 Construction of RAAV Vector Plasmids

pTR-POMC encodes murine POMC cDNA (Uhler and Herbert, 1983) under the control of the hybrid cytomegalovirus immediate early enhancer/chicken β-actin (CBA) promoter (Daly et al., 2001). The woodchuck hepatitis virus post-transcriptional regulatory element (WPRE) is placed downstream of the POMC transgene to enhance its expression (Loeb et al., 1999). The control plasmid, termed pTR-Control, is similar to pTR-POMC except for the incorporation of the cDNA encoding an enhanced form of green fluorescent protein (GFP) instead of POMC cDNA. The control vector was described previously (Li et al., 2002).

### 5.3.1.2 Packaging of RAAV Vectors

The plasmids pTR-POMC and pTR-Control were packaged in serotype 5 AAV capsids. Serotype 5 was produced by the process known as “pseudotyping” by using the helper plasmid pXYZ5 (Zolotukhin et al., 2002), containing the AAV5 capsid genes. Vectors were packaged, purified, concentrated, and titered as described (Zolotukhin et al., 2002). The titers for both rAAV-POMC and rAAV-Control vectors used in this example were 2.51×10¹³ physical particles/ml.

### 5.3.1.3 Animals

Twenty-two-month old male F344/BN rats were obtained from Harlan Sprague-Dawley (Indianapolis, Ind.) under contract with the National Institute on Aging. Upon arrival, rats were examined and remained in quarantine for one week. Animals were cared for in accordance with the principles of the NIH Guide to the Care and Use of Experimental Animals. Rats were housed individually with a 12:12 hr light:dark cycle (0700 to 1900 hr). Standard Purina 5001 rodent diet and water were provided ad libitum.

### 5.3.1.4 RAAV Vector Administration

Under 6 mg/kg xylazine (Phoenix Pharmaceutical, St. Joseph, Mo.) and 60 mg/kg ketamine (Monarch Pharmaceuticals, Bristol, TN) anesthesia, rats were administered bilaterally (2.51×10¹⁰ particles/injection in 1 μl) of rAAV-POMC (n=6) or rAAV-Control (n=6) into the basomedial hypothalamus with coordinates targeting the arcuate nucleus. The coordinates for injection into the hypothalamus were 3.14 mm posterior to bregma, ±0.4 mm lateral to the midsagittal suture and 10 mm ventral from the skull surface. On each side a small hole was drilled through the skull. Using an UltraMicropump II system (World Precision Instruments, Sarasota, Fla.), a 1-μl volume of virus stocks was delivered over 5 min to each site. The needles remained in place at the injection site for 5 additional min. At the time of surgery, rats were injected with the analgesic Buprenex (0.05 mg/kg; Reckitt and Colman, Richmond, Va.).

### 5.3.1.5 Intraperitoneal Glucose Tolerance Test (IPGTT)

IGPTT was performed at day 37 after vector administration. Rats were fasted overnight by removing their food immediately before lights out (1900 hr) and injecting them intraperitoneally with glucose (2 g/kg body weight) at 1000 hr. Blood was taken from the tail vein immediately before glucose injection, and again at 15, 30, 60, and 120 min post-injection. Blood glucose was measured by the use of One Touch SureStrep™ glucose meter (LifeScan, Milpitas, Calif.). Plasma insulin concentrations during IPGTT were also measured by a rat Insulin ELISA kit (Linco Research, St. Charles, Mo.).

### 5.3.1.6 Tissue Harvesting and Prepataion

Rats were sacrificed by cervical dislocation under 85 mg/kg pentobarbital anesthesia. Blood samples were collected by heart puncture and serum was harvested by a 15-min centrifugation in serum separator tubes. The circulatory system was perfused with 30 ml of cold phosphate-buffered saline (PBS), BAT, perirenal, retroperitoneal and epididymal white adipose tissues (PWAT, RTWAT and EWAT) were excised. The hypothalamus was removed by making an incision medial to piriforn lobes, caudal to the optic chiasm and anterior to the cerebral crus to a depth of 2-3 mm. Tissues were stored at −80° C. until analysis. For Western analysis, hypothalamus and BAT were homogenized in 0.3 ml 10 mM Tris-HCl, pH 6.8, 2% SDS, and 0.08 μg/ml okadaic acid. Protease inhibitors, 1 mM phenylmethylsulfonyl fluoride (PMSF), 0.1 mM benzamidine, and 2 μM leupeptin were also present. Homogenates were immediately boiled for 2 min, cooled on ice, and stored frozen at −80° C. Protein was determined using the DC protein assay kit (Bio-Rad, Hercules, Calif.). BAT samples were filtered through a 0.45 μm syringe filter (Whatman, Clifton, N.J.) to remove lipid particles prior to protein measurements.

### 5.3.1.7 Immunohistochemistry for GFP

Four additional rats were injected with rAAV-Control unilaterally for immunolabeling of brain sections for GFP. Anesthetized animals were perfused with 100 ml of cold PBS, followed by 400 ml of cold 4% paraformaldehyde in PBS. Thereafter, the brain was removed and equilibrated in a cryoprotectant solution of 30% sucrose/PBS and stored at 4° C. Coronal sections (50 μm) were processed free-floating for immunofluorescence. After blocking in PBS with 10% rabbit serum for 30 min, sections were incubated in an Alex Fluo 488-conjugated antibody against GFP (Molecular Probes, Eugene, Oreg., 1:750 dilution) for one hour at room temperature. The tissues then were washed in PBS/tween (0.1%) for four times. After the final wash, brain slices were mounted onto glass slides, dried, and covered with fluorescent mounting medium (Vector Laboratories, Burlingame, Calif.) for viewing with a fluorescence microscope.

### 5.3.1.8 Serum Leptin, Insulin, Glucose, Free Fatty Acids, and Cholesterol

Serum leptin was measured using a rat radioimmunoassay kit (Linco Research). Serum free fatty acids (FFAs), triglyceride and total cholesterol levels were determined by enzymatic colorimetric kits from WAKO Chemicals USA (Richmond, Va.).

### 5.3.1.9 Reverse Transcriptase-Polymerase Chain reaction (RT-PCR™)

POMC, neuropeptide Y (NPY), agouti-related protein (AgRP), MC3 receptor (MC3R), MC4 receptor (MC4R) expression levels in the hypothalamus were identified by relative quantitative RT-PCR™ using QuantumRNA 18S Internal Standards kit (Ambion, Austin, Tex.). Total cellular RNA was extracted as described (Li et al., 2002) and treated with RNase-free DNase (Ambion). First-strand cDNA synthesis was generated from 2 μg RNA in a 20-μl volume using random primers (Life Technologies, Rockville, Md.) containing 200 units of M-MLV reverse transcriptase (Life Technologies). Relative quantitative PCR was performed by multiplexing corresponding primers:

| | |
|---|---|
| POMC | |
| sense | |
| 5′-GCTTGCAAACTCGACCTCTC-3′, | (SEQ ID NO. 45) |
| antisense | |
| 5′-CTTGATGATGGCGT TCTTGA-3′; | (SEQ ID NO: 46) |
| NPY | |
| sense | |
| 5′-ATGGGGCTGTGTGGACTGACC-3′, | (SEQ ID NO: 47) |
| antisense | |
| 5′-GTCAGGAGAGCAAGTTTCATTT-3′, | (SEQ ID NO: 48) |
| AgRP | |
| sense | |
| 5′-AGGGCATCAGAAGGCCTGACCA-3′, | (SEQ ID NO: 49) |
| antisense | |
| 5′-CTTGAAGAAGCGGCAGTAGCAC-3′; | (SEQ ID NO: 50) |
| MC3R | |
| sense | |
| 5′-AGCAACCGGAGTGGCAGT-3′; | (SEQ ID NO: 51) |
| antisense | |
| 5′-GGCCACGATCAA GGAGAG-3′; | (SEQ ID NO: 52) |
| MC4R | |
| sense | |
| 5′-AGTCTCTGGGGAAGGGGCA-3′; | (SEQ ID NO: 53) |
| antisense | |
| 5′-CAACTGATGATGATCCCGAC-3′, | (SEQ ID NO 54) |

18S primers, and competimers and coamplifying. Linearity for all amplicons was determined to be between 20 and 29 cycles. The optimum ratio of 18S primer to competimer was 1:5 for POMC, 1:7 for NPY, 1:4 for AgRP, 1:6 for MC3R, and 1:9 for MC4R. PCR was performed at 94° C. denaturation for 60 sec, 59° C. annealing temperature for 50 sec, and 72° C. elongation temperature for 50 sec for 26 (POMC), 22 (NPY), 25 (AgRP), 23 (MC3R) or 27(MC4R) cycles. The PCR product was electrophoresed on acrylamide gel and stained with SYBR green (Molecular Probes, Eugene, Oreg.). Gels were scanned using a STORM fluorescent scanner and data analyzed using ImageQuant (Molecular Dynamics). The relative value of each mRNA was derived from dividing the signal obtained for corresponding amplicon by that for 18S amplicon.

### 5.3.1.10 UCP1 Protein

Immunoreactive UCP1 was determined with an antibody specific to rat UCP1 (Linco Research). BAT homogenates (20 μg) prepared as described above were separated on a SDS-PAGE gel and electrotransferred to nitrocellulose membrane. Immunoreactivity was assessed with an antibody specific to UCP1. Immunoreactivity was visualized by enhanced chemiluminescent detection (Amersham Pharmacia Biotech) and quantified by video densitometry (Bio-Rad).

### 5.3.1.11 Statistical Analysis

Results are presented as mean ±SE. Repeated-measure ANOVA was used for analyses of body weight and food intake. When the main effect was significant, a post-hoc test was applied to determine individual differences between means. For all other data, unpaired two-tailed Student's t-test was employed. A value of P<0.05 was considered significant.

### 5.3.2 RESULTS

### 5.3.2.1 Immunohistochemical Localization of GFP

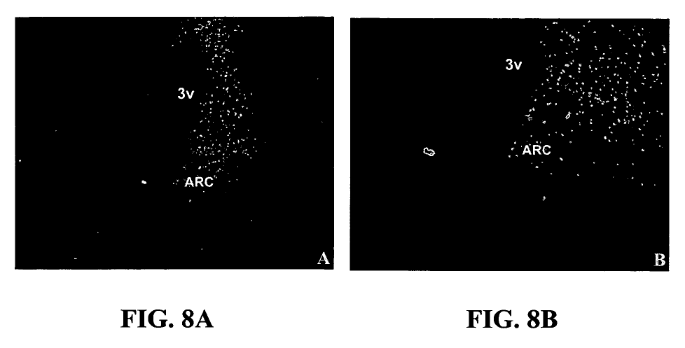

A GFP reporter gene was incorporated into the control vector for easy identification of cells transfected with serotype 5 rAAV. The coordinates used in this study target the arcuate nucleus of the hypothalamus, and the anatomic localization of hypothalamic cells transfected with rAAV was investigated by immunolabeling of GFP in brain sections of rats given unilateral injection of rAAV-Control. GFP positive cells were distributed around the tract of the injection needle, but mainly at the tip of injection needle, that is, the basomedial posterior hypothalamus, especially the arcuate nucleus. In contrast, no GFP immunoreactive cells were observed in the contralateral side of the hypothalamus (FIG. 8A and FIG. 8B).

### 5.3.2.2 POMC Expression in the Hypothalamus of Obese Aged Rats

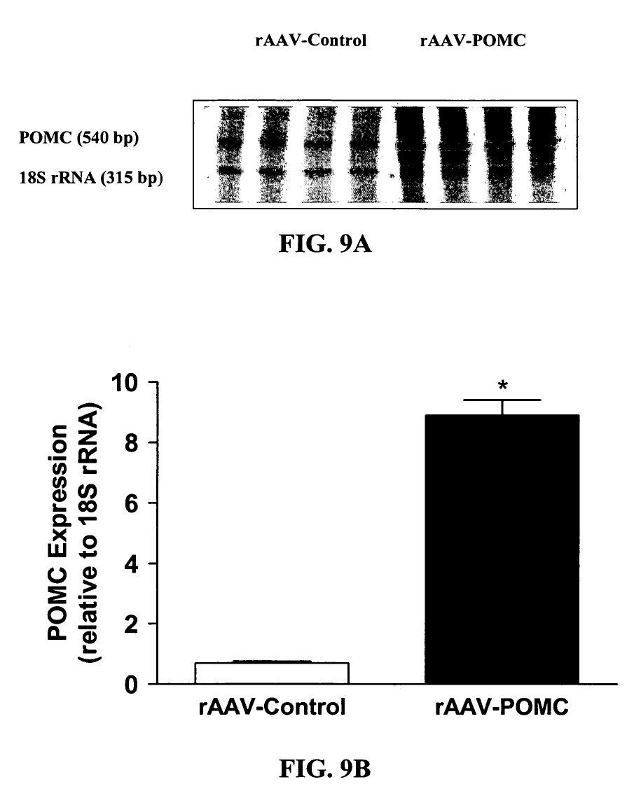

To verify overexpression of the POMC transgene following central bilateral viral delivery, POMC mRNA was measured in the hypothalamus by RT-PCR (FIG. 9A and FIG. 9B). Forty two days after vector delivery, hypothalamic POMC mRNA levels were elevated by nine-fold in obese aged F344/BN rats given rAAV-POMC as compared to those given rAAV-Control (p<0.001).

### 5.3.2.3 Food Intake and Body Weight

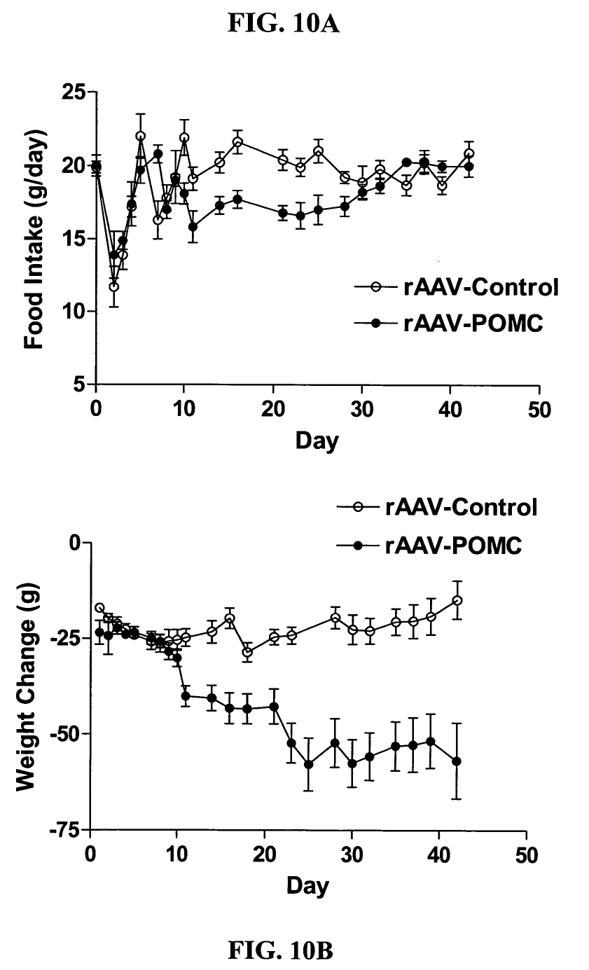

Bilateral delivery of rAAV-POMC into the basomedial hypothalamus resulted in a sustained reduction in body weight and a transient (19 days) suppression of food intake in aged obese F344/BN rats (FIG. 10A and FIG. 10B). Both rAAV-POMC and rAAV-Control-treated rats had transient anorexia after surgical administration of vectors but completely recovered their food intake from day 5 after vector injection. POMC gene delivery significantly reduced food consumption from day 10 after vector injection, and these rats ate 2-4 g less per day compared to control rats. However, this anorexia attenuated by day 28 (FIG. 10A). In contrast, there was a steady decrease in body weight with no apparent attenuation following the POMC gene delivery (FIG. 10B). Before and on the day of vector delivery, average body weight of rAAV-POMC treated rats was comparable to that of control rats (591±23 vs. 569±18 g at day 0, P=0.5). After vector delivery, both rAAV-POMC and rAAV-Control-treated rats lost about 20 to 25 g of body weight due to surgical intervention. While body weight of control rats remained steady throughout the whole experimental period, rats given rAAV-POMC lost more weight starting from day 10, and the difference in body mass between the two groups gradually increased over the forty-two days (FIG. 10B). At the end of the experiment (day 42), rAAV-POMC rats lost an average of 9.5% of their initial body weight as compared to only 2.7% in control rats (−56.8±9.9 vs. −14.8±5.0 g of weight change, P<0.01).

### 5.3.2.4 Viscera; Adiposity and Serum Leptin Levels

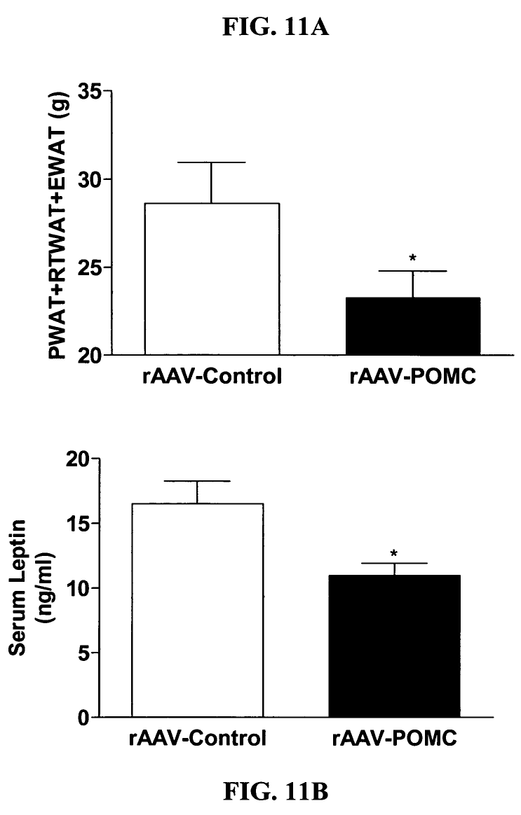

Because POMC gene therapy reduced the body weight of the aged obese rats, body adiposity levels were assessed. Forty-two days after central POMC gene delivery, there were significant reductions in the visceral adiposity, as reflected by a 19% reduction in the sum of the PWAT, RTWAT and EWAT (P<0.05) in rAAV-POMC-treated compared to control rats (FIG. 11A). Serum leptin levels, one indicator of body fat mass (Frederich et al., 1995), were 33% lower in the rAAV-POMC group compared to the control group (FIG. 11B).

### 5.3.2.5 Intraperitoneal Glucose Tolerance Test

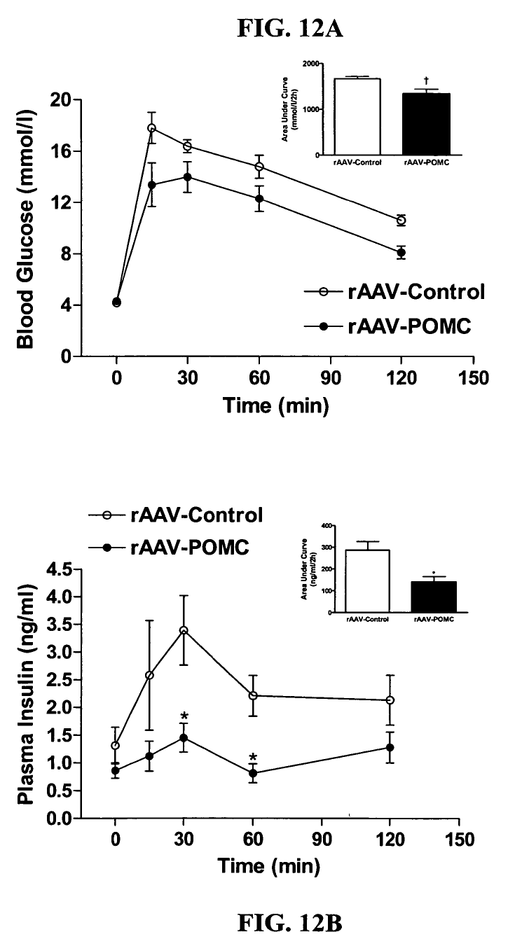

Mounting evidence supports a role for the central MC system in the regulation of glucose metabolism, thus, we performed an IPGTT at day 37 after vector injection. As shown in FIG. 12A, although the fasting blood glucose levels of rAAV-Control and rAAV-POMC-treated rats were similar, POMC gene delivery significantly accelerated glucose clearance after glucose administration (P<0.05 by repeated ANOVA). The capacity to metabolize glucose in these aged obese rats, as indicated by the area under the curve in the IPGTT, was improved by 19% following rAAV-POMC treatment. Consistent with these findings, plasma insulin levels at each time point were reduced in rAAV-POMC-treated rats compared with rAAV-Control rats following the glucose challenge (FIG. 12B).

### 5.3.2.6 Serum FFA, Triglyceride, and Cholesterol

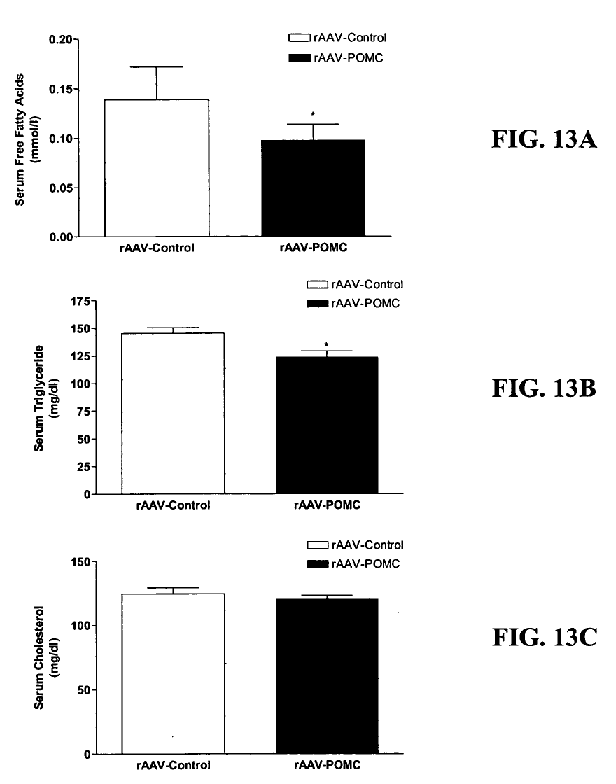

Forty-two days of POMC gene delivery reduced serum free fatty acids and triglyceride by 30% and 15%, respectively (FIG. 13A and FIG. 13B). Meanwhile, serum cholesterol levels were comparable between rAAV-POMC and control groups (FIG. 13C).

### 5.3.2.7 Brown Adipose Tissue (BAT)

Induction of UCP1 in BAT is an important marker for enhanced thermogenesis and thus, energy expenditure in rodents (Scarpace et al., 1997; Cannon and Nedergaard, 2004). UCP1 protein levels were examined 42 days after POMC gene delivery. Total BAT weight markedly declined with rAAV-POMC treatment whereas the protein concentration (per unit BAT) increased dramatically, suggesting the reduction in BAT mass was due to the lipolysis associated with the activation of BAT. This was further supported by a 71% increase in BAT UCP1 protein levels in the rAAV-POMC-treated compared with control rats (Table 5).

TABLE 5
UNCOUPLING PROTEIN 1 AND BAT PARAMETERS
42 DAYS FOLLOWING RAAV-POMC OR RAAV-
CONTROL DELIVERY

| | Treatment | |
|---|---|---|
| | rAAV-Control | rAAV-POMC |
| BAT weight (mg) | 689 ± 28 | 488 ± 39** |
| BAT protein (mg/g BAT) | 67.3 ± 3.5 | 92.5 ± 4.8** |
| BAT protein (mg/total BAT) | 46.2 ± 2.8 | 44.5 ± 2.7 |
| UCP1 protein (arbitrary units/g BAT) | 100 ± 6 | 171 ± 22* |
| UCP1 protein (arbitrary units/ | 100 ± 11 | 155 ± 16* |
| total BAT) | | |

For UCP1 protein, the levels in rAAV-Control rats are set to 100 and SE adjusted proportionally. Data represent the mean ± SE of 6 rats per group.
*P < 0.05 and **P < 0.01 vs. control by unpaired t-test.

### 5.3.2.8 Hypothalamic NPY, AGRP, MC3R, and MC4R mRNA Levels

To assess the effects of POMC gene delivery on the expression of hypothalamic neuropeptides, NPY and AgRP MRNA levels were measured 42 days after POMC vector delivery (Table 6). RT-PCRTM revealed that the expression of AgRP, the endogenous antagonist of MC receptors, was unchanged in rats given rAAV-POMC as compared with control rats, whereas the mRNA levels of another potent orexigenic neuropeptide, NPY tended toward a decrease (P=0.14).

MC3 and MC4 receptors are the predominant MC receptors in the hypothalamus and mediate the effects of POMC-derived α-melanocyte stimulating hormone (α-MSH) on the homeostatic regulation of body weight. The delivery of rAAV-POMC reduced the expression levels of hypothalamic MC3R and MC4R by 25% (P <0.05) and 17% (P=0.09), respectively, compared with rAAV-Control (Table 6).

TABLE 6
HYPOTHALAMIC NEUROPEPTIDES, MELANOCORTIN 3
(MC3R) AND 4 RECEPTORS 42 DAYS FOLLOWING
RAAV-POMC OR RAAV-CONTROL DELIVERY

Treatment

| | rAAV-Control | rAAV-POMC |
|---|---|---|
| NPY | 1.6380 ± 0.1189 | 1.3731 ± 0.1136 |
| AgRP | 0.5242 ± 0.0461 | 0.5308 ± 0.0370 |
| MC3R | 0.5985 ± 0.470 | 0.4964 ± 0.0285 |
| MC4R | 0.7738 ± 0.572 | 0.5785 ± 0.0353* |

All mRNA levels were measured by relative quantitative RT-PCR ™ with 18S rRNA as an internal standard. The relative value of each mRNA was derived from dividing the signal obtained for corresponding amplicon by that for 18S amplicon. Data represent the mean ± SE of 6 rats per group.
*P < 0.05 vs. control by unpaired t-test.

### 5.3.3 DISCUSSION

The present study examined the long-term consequences of central rAAV-mediated POMC gene therapy in aged obese F344/BN rats. The findings are in agreement with earlier short-term pharmacological study indicating that direct activation of the central MC system by the α-MSH analogue MTII is effective in partially reversing the obese phenotype in aged rats (Zhang et al., 2004). The present approach, in particular, resulted in long-term overexpression of POMC in the basal hypothalamus using serotype 5 rAAV, and these aged rats responded with significant reductions in both food intake and body weight. This is the first study reporting the effectiveness of gene delivery into the hypothalamus using serotype 5 AAV. To date, eight serotypes of primate AAV have been identified (Gao et al, 2002). Of these, type 2 AAV is best characterized and most employed in gene therapy studies. Type 5 AAV has a distinct advantage over type 2 due to a higher level of transduction efficiency in certain tissues (Hildinger et al., 2001; Zabner et al., 2000). Observation of a robust transgene POMC expression in the hypothalamus following type 5 rAAV-POMC delivery suggests that type 5 rAAV is a viable option for long-term gene delivery in the hypothalamus.

The present study provides several distinct sets of salient findings. First, in aged obese rats, rAAV-mediated POMC gene delivery results in a sustained reduction in body weight despite a short-term suppression in food consumption. The decrease in food intake in rAAV-POMC-treated rats commenced at day 10 after vector injection and abated on day 29 and afterward. Meanwhile, starting from day 10 after vector administration, rats given rAAV-POMC consistently lost more body weight when compared with rats given rAAV-Control. For the most part the greatest decrease in body weight occurred during the period when food intake was diminished. Soon after the anorexia attenuated, the average body weight of POMC vector-treated rats reached a nadir and then stabilized. The absence of a rebound in body weight in rAAV-POMC-treated animals after the attenuation of anorexia suggests that another component of body weight homeostasis, energy expenditure might be persistently elevated in response to central POMC gene therapy. It has been previously demonstrated that an increase in energy expenditure is sufficient to prevent the regain in body weight following anorexia (Scarpace et al., 2002b). In this example, the inventors have shown that UCP1 protein levels were significantly elevated 42 days after POMC vector delivery, and 2 weeks after the attenuation of the POMC-mediated inhibition in food intake. UCP1-mediated nonshivering thermogenesis in BAT represents an essential element in adaptive energy expenditure in rodents. An increase in UCP1 protein is indicative of increased BAT-facilitated energy expenditure (Scarpace et al., 1997; Cannon and Nedergaard, 2004). It is known that pharmacological activation of MC system augments energy expenditure in rodents. For example, normal animals treated with the a-MSH analog, MTII, have elevated levels of BAT UCP1 expression compared with pair-fed controls (Cettour-Rose and Rohner-Jeanrenaud, 2002). In Example 5.2, a serotype 2 rAAV-POMC vector also markedly stimulated BAT thermogenesis in obese Zucker rats at 38 days after vector delivery (Li et al., 2003). Although lacking direct evidence of whole body energy expenditure, we speculate based on the increase in UCP1 protein that, in addition to the hypophagia, an increase in energy expenditure contributed to the amelioration of body weight and fat in aged-obese rats following central POMC gene therapy, and in particular, was instrumental in maintaining the lost weight after the anorexia attenuated.

Second, although the 19-day anorexic response to POMC gene delivery attenuated, the onset of this tachyphylaxis to central POMC gene delivery was markedly delayed as compared with attenuation of the anorexic response following pharmacological administration of α-MSH or MTII in either normal or dietary obese mice and rats (McMinn et al., 2000; Pierroz et al., 2002). The suppression of food consumption lasted no longer than four days in any of these latter studies. Additionally, tachyphylaxis to MTII occurred within 6 days following central MTII infusion in aged obese F344/BN rats (Zhang et al., 2004). The mechanism for the rapid attenuation to melanocortin treatment in pharmacological studies is not clear, but may involve agonist-mediated receptor internalization (Shinyama et al., 2003). In contrast to these pharmacological studies, AAV mediated POMC gene therapy suppressed food intake for up to 38 days in obese Zucker rats (Li et al., 2003), and in the present example, for 19 days in aged-obese rats. Therefore, the prolonged anorexic response observed in aged and obese Zucker rats may be unique to central POMC gene delivery. Considering that POMC is only expressed at two locations in the brain: the arcuate nucleus of the hypothalamus and the nucleus of the tractus solitarius of the brainstem, POMC vector was delivered into the basal medial hypothalamus aiming at the arcuate nucleus where POMC-expressing neurons are located. With this procedure, the overproduction of α-MSH derived from transgene POMC expression is presumably assisted by a variety of endogenous enzymes such as prehormone convertases, carboxypeptidase E and peptidyl a-amidating mono-oxygenase (Pritchard et al., 2002). This endogenously regulated production of α-MSH may help prevent the rapid desensitization witnessed in previous pharmacological studies. The prolonged anorexic response could also be attributed to potential overproduction of other peptides in addition to α-MSH, such as β-MSH and β-endorphin, both of which are normally derived from the POMC precursor. It has been suggested that these peptides also participate in the regulation of energy balance (Appleyard et al., 2003; Harrold et al, 2003).

Central POMC gene therapy also appears to impact the expressions of the two important central melanocortin receptors. When assessed at sacrifice, the hypothalamic MC4R expression was significantly reduced while MC3R MRNA levels tended to decrease in the rAAV-POMC rats. Interestingly, the expressions of the two orexigenic neuropeptides, NPY and AgRP did not change after POMC gene delivery. These data suggest that prolonged exposure to POMC gene over-expression in the aged animals may down-regulate hypothalamic melanocortin receptors. This may be one desensitization mechanism for the attenuation of POMC gene therapy-evoked anorexia.

Third, rAAV-POMC gene delivery improves glucose metabolism and insulin sensitivity in aged obese rats. Aged F344/BN rats are associated with insulin resistance and glucose intolerance as shown in several previous reports (Gabriely et al., 2002; Larkin et al., 2001; Gupta et al., 2000). In this example, the impaired glucose tolerance was observed as indicated by elevated blood glucose levels after glucose loading in the aged obese F344/BN rats. Central POMC gene therapy partially normalized glucose levels during IPGTT, and also reduced serum insulin levels markedly at all time points following glucose loading. These data are indicative of improved glucose metabolism and insulin sensitivity by POMC gene delivery and in agreement with previous findings that central MC receptor activation suppresses insulin release from pancreas and enhances glucose metabolism (Fan et al., 1997; Obici et al., 2001; Li et al., 2003). Since POMC gene therapy also significantly reduced visceral adiposity in these aged obese rats, this loss in visceral adiposity could also contribute to the improved glucose metabolism. In addition to its impact on insulin and glucose, POMC gene delivery also reduced serum triglyceride and FFA levels in the obese aged rats. Such effects could be due to the reduced lipogenesis and/or increased lipolysis in white fat tissues along with the enhanced non-shivering thermogensis in BAT following POMC gene therapy.

In conclusion, targeted POMC gene delivery to the hypothalamus suppressed food intake, diminished body weight and reduced visceral adiposity in aged obese rats. This treatment also improved glucose and fat metabolism and insulin sensitivity. The induced hypophagia and stimulated BAT thermogensis are the likely mechanisms underlying these improvements, and these data suggest that long-term POMC over-expression is a viable strategy to combat age-related obesity.

### 5.4 Example 4

### Pro-Opiomelanocortin Gene Delivery in the Hypothalamus Reduces Body Weight and Improve Glucos Metabolim in Aged Obese Rats

In this example, the effect of POMC gene therapy on age-related obesity has been demonstrated. rAAV-POMC or control vectors (5.02×10¹⁰ particles/rat) were delivered bilaterally into the basal hypothalamus of 22-month old male F344/BN rats with coordinates targeting the arcuate nucleus. Assessed at day 42 after POMC gene delivery, hypothalamic POMC expression increased by 10-fold while AgRP and NPY MRNA levels remained unchanged. POMC gene delivery reduced food consumption from day 10 post vector injection, but this anorexic effect attenuated by day 28. In contrast, there was a steady decrease in body weight with no apparent attenuation. Visceral adiposity decreased by 19% (P<0.02), and as indicated by intra-peritoneal glucose tolerance test, glucose metabolism and insulin sensitivity improved by 19% (P=0.02) in rAAV-POMC compared with controls. Serum free fatty acid and triglycerol levels were also significantly reduced by rAAV-POMC treatment. POMC gene delivery enhanced uncoupling protein 1 in brown adipose tissue (BAT) by more than 70%. Hypothalamic MC4 and MC3 receptor expression decreased by 25% (P>0.02) and 17% (P=0.1) in rAAV-POMC rats, respectively.

These data demonstrate that targeted POMC gene delivery in the hypothalamus reduces body weight and visceral adiposity, and improves glucose and fat metabolism in aged obese rats. The transient anorexia and stimulated BAT thermogenesis may contribute to these effects. The down-regulation of hypothalamic MC receptor expression following POMC gene delivery may be the consequence of prolonged exposure to POMC over-expression and could be one underlying mechanism for the attenuation of the anorexic response to POMC gene delivery in aged obese animals.

### 5.5 Example 5

### POMC Polypeptides and POMC-Encoding Nucleic Acid Segments

The inventors contemplate the use of one or more of the pro-opiomelanocortin polypeptide and DNA sequences illustrated in Table 7, and disclosed in SEQ ID NO: 1 through SEQ ID NO:40 in the preparation of rAAV vector-based constructs in the practice of the present invention. In illustrative embodiments, the inventors contemplate the use of mammalian pro-opiomelanocortin-encoding polynucleotides, and in particular, the use of human pro-opiomelanocortin-encoding polynucleotides in creation of rAAV vector constructs for use in the preparation of medicaments, and in prophylactic and/or therapeutic regimens as described herein.

TABLE 7
EXEMPLARY PRO-OPIOMELANOCORTIN POLYPEPTIDE
SEQUENCES AND POLYNUCLEOTIDE SEQUENCES
ENCODING POMC POLYPEPTIDES USEFUL IN THE
PRACTICE OF THE PRESENT INVENTION

| | GENBANK/SWISSPROT | |
|---|---|---|
| MOLECULE | ACCESSION NUMBERS | SEQ ID NOS: |
| Human POMC | NM_000939 | SEQ ID NO: 1 |
| | NM_000939.1 | SEQ ID NO: 2 |
| Gorilla POMC | AY091992 | SEQ ID NO: 3 |
| | AAM76609 | SEQ ID NO: 4 |
| Pig-Tailed Macaque | M19658 | SEQ ID NO: 5 |
| POMC | P01201 | SEQ ID NO: 6 |
| Orangutan POMC | AY091993 | SEQ ID NO: 7 |
| | AAM76610 | SEQ ID NO: 8 |
| Porcine POMC | X00135; X03561 | SEQ ID NO: 9 |
| | AAB32312 | SEQ ID NO: 10 |
| Bovine POMC | NM_174151 | SEQ ID NO: 11 |
| | | SEQ ID NO: 12 |
| Canine POMC | AY024339 | SEQ ID NO: 13 |
| | AAK08973 | SEQ ID NO: 14 |
| Guinea Pig POMC | S78260 | SEQ ID NO: 15 |
| | A54322 | SEQ ID NO: 16 |
| Rat POMC | AH002232 | SEQ ID NO: 17 |
| | NP_647542 | SEQ ID NO: 18 |
| Murine POMC | AH005319 | SEQ ID NO: 19 |
| | | SEQ ID NO: 20 |
| Chicken POMC | A54322 | SEQ ID NO: 21 |
| | BAA34366 | SEQ ID NO: 22 |
| Giant Toad | AAF06345 | SEQ ID NO: 23 |
| | | SEQ ID NO: 24 |
| Carp POMC | Y14618 | SEQ ID NO: 25 |
| | | SEQ ID NO: 26 |
| Danio rerio POMC | AY125332 | SEQ ID NO: 27 |
| | AY135148 | SEQ ID NO: 28 |
| Bullfrog POMC | X15510 | SEQ ID NO: 29 |
| | | SEQ ID NO: 30 |
| Opossum POMC | AAL13338 | SEQ ID NO: 31 |
| Sheep POMC | CAD45184 | SEQ ID NO: 32 |
| Sheep Partial | P01191 | SEQ ID NO: 33 |
| POMC Var. 1 | | |
| Giant Bullfrog POMC | P11885 | SEQ ID NO: 34 |
| Mexican Spadefoot | AAD21040 | SEQ ID NO: 35 |
| Toad POMC | | |
| African Clawed | P06298 | SEQ ID NO: 36 |
| Frog POMC | | |
| Mudpuppy POMC | AAN46359 | SEQ ID NO: 37 |
| Amphiuma POMC | AAN46358 | SEQ ID NO: 38 |
| Softshell Turtle POMC | AAM34798 | SEQ ID NO: 39 |
| Chimpanzee POMC | AAM76608 | SEQ ID NO: 40 |

| | |
|---|---|
| 5.5.1 HUMAN (HOMO SAPIENS) | |
| POMC DNA (FROM GENBANK NM_000939) | |
| ATGCCGAGATCGTGCTGCAGCCGCTCGGGGGCCC | (SEQ ID NO: 1) |
| TGTTGCTGGCCTTGCTGCTTCAGGCCTCCATGGA | |
| AGTGCGTGGCTGGTGCCTGGAGAGCAGCCAGTGT | |
| CAGGACCTCACCACGGAAAGCAACCTGCTGGAGT | |
| GCATCCGGGCCTGCAAGCCCGACCTCTCGGCCGA | |
| GACTCCCATGTTCCCGGGAAATGGCGACGAGCAG | |
| CCTCTGACCGAGAACCCCCGGAAGTACGTCATGG | |
| GCCACTTCCGCTGGGACCGATTCGGCCGCCGCAA | |
| CAGCAGCAGCAGCGGCAGCAGCGGCGCAGGGCAG | |
| AAGCGCGAGGACGTCTCAGCGGGCGAAGACTGCG | |
| GCCCGCTGCCTGAGGGCGGCCCCGAGCCCCGCAG | |
| CGATGGTGCCAAGCCGGGCCCGCGCGAGGGCAAG | |
| CGCTCCTACTCCATGGAGCACTTCCGCTGGGGCA | |
| AGCCGGTGGGCAAGAAGCGGCGCCCAGTGAAGGT | |
| GTACCCTAACGGCGCCGAGGACGAGTCGGCCGAG | |
| GCCTTCCCCCTGGAGTTCAAGAGGGAGCTGACTG | |
| GCCAGCGACTCCGGGAGGGAGATGGCCCCGACGG | |
| CCCTGCCGATGACGGCGCAGGGGCCCAGGCCGAC | |
| CTGGAGCACAGCCTGCTGGTGGCGGCCGAGAAGA | |
| AGGACGAGGGCCCCTACAGGATGGAGCACTTCCG | |
| CTGGGGCAGCCCGCCCAAGGACAAGCGCTACGGC | |
| GGTTTCATGACCTCCGAGAAGAGCCAGACGCCCC | |
| TGGTGACGCTGTTCAAAAACGCCATCATCAAGAA | |
| CGCCTACAAGAAGGGCGAGTGA | |
| 5.5.2 HUMAN | |
| (HOMO SAPIENS) POMC PROTEIN | |
| MPRSCCSRSGALLLALLLQASMEVRGWCLESSQC | (SEQ ID NO: 2) |
| QDLTTESNLLECIRACKPDLSAETPMFPGNGDEQ | |
| PLTENPRKYVMGHFRWDRFGRRNSSSSGSSGAGQ | |
| KREDVSAGEDCGPLPEGGPEPRSDGAKPGPREGK | |
| RSYSMEHFRWGKPVGKKRRPVKVYPNGAEDESAE | |
| AFPLEFKRELTGQRLREGDGPDGPADDGAGAQAD | |
| LEHSLLVAAEKKDEGPYRMEHFRWGSPPKDKRYG | |
| GFMTSEKSQTPLVTLFKNAIIKNAYKKGE | |
| 5.5.3 GORILLA | |
| (GORILLA GORILLA) POMC PARTIAL | |
| DNA (FROM GENBANK AY091992) | |
| CTCGGCCGAGACTCCCATGTTCCCGGGCAATGGC | (SEQ ID NO: 3) |
| GACGAGCAGCCTCTGACCGAGAACCCCCGGAAGT | |
| ACGTCATGGGCCACTTCCGCTGGGACCGATTCGG | |
| CCGCCGCAACAGCAGCAGCAGCAGCGGCAGCGGC | |
| GCAGGGCAGAAGCGCGAGGATGTCTCAGCGGGCG | |
| AAGACCGCGGCCCGCTGCCTGAGGGCGGCCCCGA | |
| GCCCCGCAGTGATGGTGCCAAGCCGGGCCCGCGC | |
| GAGGGCAAGCGCTCCTACTCCATGGAGCACTTCC | |
| GCTGGGGCAAGCCGGTGGGCAAGAAGCGGCGCCC | |
| GGTGAAGGTGTACCCTAACGGCGCCGAGGACGAG | |
| TCGGCCGAGGCCTTCCCCCTGGAGTTCAAGAGGG | |
| AGCTGACTGGCCAGCGACCCCGGGACGGAGATGG | |
| CCCCGACGGCCCTGCCGATGACGGCGCCGGGGCC | |
| CAGGCCGACCTGGAGCATAGCCTGCTGGTGGCGG | |
| CCGAGAAGAAGGACGAGGGCCCCTACGGGATGGA | |
| GCACTTCCGCTGGGGCAGCCCGCCCAAGGACAAG | |
| CGCTACGGCGGTTTC | |
| 5.5.4 GORILLA | |
| (GORILLA GORILLA) POMC PARTIAL | |
| PROTEIN (FROM GENBANK AAM76609; | |
| O'HUIGIN ET AL.) 183 AMINO ACIDS | |
| SAETPMFPGNGDEQPLTENPRKYVMGHFRWDRFG | (SEQ ID NO: 4) |
| RRNSSSSSGSGAGQKREDVSAGEDRGPLPEGGPE | |
| PRSDGAKPGPREGKRSYSMEHFRWGKPVGKKRRP | |
| VKVYPNGAEDESAEAFPLEFKRELTGQRPREGDG | |
| PDGPADDGAGAQADLEHSLLVAAEKKDEGPYGME | |
| HFRWGSPPKDKRYGGF | |
| 5.5.5 PIG-TAILED MACAQUE | |
| (MACACA NEMESTRINA) POMC DNA (FROM | |
| GENBANK M19658; O'HUIGIN ET AL.) | |
| ATGCCGAGATCGTGCTGCAGCCGCTCGGGGGCCC | (SEQ ID NO: 5) |
| TGTTGCTGGCCTTGCTGCTTCAGGCCTCCATGGA | |
| AGTGCGTGGCTGGTGCCTGGAGAGCAGCCAGTGT | |
| CAGGACCTCACCACGGAAAGCAACCTGCTGGAGT | |
| GCATCCGGGCCTGCAAGCCCGACCTTTCGGCCGA | |
| GACTCCGGTGTTTCCGGGCAATGGCGACGAGCAG | |
| CCTCTGACCGAGAACCCCCGGAAGTACGTCATGG | |
| GCCACTTCCGCTGGGACCGATTCGGCGGCCGCAA | |
| CAGTAGCAGCGGCAGCGCGCACCAGAAGCGCGAG | |
| GACGTCGCGGCTGGCGAAGACCGCGGCCTGCTAC | |
| CTGAGGGTGGCCCCGAGGCCCGTGGCGATGGCGC | |
| CGGGCCGGGCCCGCGCGAGGGCAAGCGCTCCTAC | |
| TCCATGGAGCACTTCCGCTGGGGCAAGCCGGTGG | |
| GCAAGAAGCGGCGCCCGGTGAAGGTGTACCCCAA | |
| TGGCGCCGAGGACGAGTCGGCCGAGGCCTTCCCC | |
| CTGGAGTTCAAGAGGGAGCTGACCGGCCAGCGGC | |
| CCCGGGCGGGGGATGGCCCCGATGGCCCTGCCGA | |
| CGACGGCGCGGGGCCCCGGGCCGACCTGGAGCAC | |
| AGCCTGCTGGTGGCGGCCGAGAAGAAGGATGAGG | |
| GCCCCTACAGGATGGAGCACTTCCGCTGGGGCAG | |
| CCCGCCCAAGGACAAGCGGTACGGCGGCTTGATG | |
| ACCTCCGAGAAGAGCCAGACTCCCCTGGTGACAC | |
| TGTTCAAAAACGCCATCATCAAGAACGCGTACAA | |
| GAAGGGCCAGTGA | |
| 5.5.6 PIG-TAILED MACAQUE (MACACA | |
| NEMESTRINA) POMC PROTEIN (FROM | |
| SWISSPROT P01201; O'HUIGIN ET AL.) | |
| MPRSCCSRSGALLLALLLQASMEVRGWCLESSQC | (SEQ ID NO: 6) |
| QDLTTESNLLECIRACKPDLSAETPVFPGNGDEQ | |
| PLTENPRKYVMGHFRWDRFGRRNSSSGSAHQKRE | |
| DVAAGEDRGLLPEGGPEPRGDGAGPGPREGKRSY | |
| SMEHFRWGKPVGKKRRPVKVYPNGAEDESAEAFP | |
| LEFKRELTGQRPRAGDGPDGPADDGAGPRADLEH | |
| SLLVAAEKKDEGPYRMEHFRWGSPPKDKRYGGFM | |
| TSEKSQTPLVTLFKNAIIKNAYKKGQ | |
| 5.5.7 ORANGUTAN (PONGO PYGMAEUS) | |
| POMC DNA (FROM GENBANK AY091993) | |
| GAAGTACGTCATGGGCCACTTCCGCTGGGACCGA | (SEQ ID NO: 7) |
| TTTGGCCGCCGCAACAGCAGCAGCGGCAGCGGTA | |
| GCGGCGCAGGGCAGAAGCGCGAGGACGTCGCAGC | |
| GGGCGAAGACCGCGGCCCACTGCCTGAGGGCGGC | |
| CCCGAGCCCCGCAGCGATGGCGCCGAGCCGGGCC | |
| CGCGCGAGGGCAAGCGCTCCTACTCCATGGAGCA | |
| CTTCCGCTGGGGCAAGCCGGTGGGCAAGAAGCGG | |
| CGCCCGGTGAAGGTGTACCCCAACGGCGCCGAGG | |
| ACGAGTCGGCCGAGGCCTTCCCCCTGGAGTTCAA | |
| GAGGGAGCCGACCGGCCAGCGGCTCCGGGAGGGA | |
| GATGGCCCCGACGGCCCTGCCGATGACGGCGCCG | |
| GGGCCCGGGCCGACCTGGAGCACAACCTGCTGGT | |
| GGCGGCCGAGAAGAAGGACGAGGGCCCCTACAGG | |
| ATGGAGCACTTCCGCTGGGGCAGCCCGCCCAAGG | |
| ACAAGCGCTACGGCGGTTTC | |
| 5.5.8 ORANGUTAN | |
| (PONGO PYGMAEUS) PARTIAL POMC | |
| PROTEIN (FROM GENBANK AAM76610; | |
| O'HUIGIN ET AL.) 165 AMINO ACIDS | |
| KYVMGHFRWDRFGRRNSSSGSGSGAGQKREDVAA | (SEQ ID NO: 8) |
| GEDRGPLPEGGPEPRSDGAEPGPREGKRSYSMEH | |
| FRWGKPVGKKRRPVKVYPNGAEDESAEAFPLEFK | |
| REPTGQRLREGDGPDGPADDGAGARADLEHNLLV | |
| AAEKKDEGPYRMEHFRWGSPPKDKRYGGF | |
| 5.5.9 PORCINE (SUS SCROFFA) | |
| POMC DNA (FROM GENBANK X00135) | |
| ATGCCGAGATTGTGCGGCAGTCGCTCGGGGGCCC | (SEQ ID NO: 9) |
| TCCTGCTGACCTTGCTGCTCCAGGCCTCCATGGG | |
| AGTGCGCGGCTGGTGCTTGGAGAGCAGCCAGTGT | |
| CAGGACCTCTCCACGGAAAGTAACTTGTTGGCGT | |
| GCATCCGGGCCTGCAAACCAGATCTCTCTGCGGA | |
| GACGCCCGTGTTTCCCGGCAACGGCGACGCGCAA | |
| CCGCTGACCGAGAACCCCCGGAAGTACGTCATGG | |
| GCCACTTCCGCTGGGACCGCTTCGGCCGCCGGAA | |
| TGGCAGCAGCAGCGGCGGCGGTGGCGGTGGCGGC | |
| GGCGCGGGCCAGAAGCGCGAGGAGGAGGAGGTGG | |
| CGGCGGGCGAAGGCCCCGGGCCCCGCGGAGATGG | |
| CGTCGCGCCGGGCCCGCGCCAGGACAAGCGCTCC | |
| TACTCCATGGAGCACTTCCGCTGGGGCAAGCCCG | |
| TGGGCAAGAAGCGGCGCCCGGTGAAGGTGTATCC | |
| CAACGGCGCCGAGGACGAGTTGGCCGAGGCCTTC | |
| CCCCTCGAGTTCAGGAGGGAGCTGGCCGGGGCGC | |
| CCCCCGAGCCGGCACGGGACCCCGAGGCCCCGGC | |
| CGAGGGCGCGGCCGCCCGGGCCGAGCTGGAGTAC | |
| GGGCTGGTGGCCGAGGCCGAGGCGGCCGAGAAGA | |
| AGGACGAAGGGCCCTATAAGATGGAGCACTTCCG | |
| CTGGGGCAGCCCGCCCAAGGACAAGCGCTACGGC | |
| GGCTTCATGACCTCCGAGAAGAGCCAGACGCCCC | |
| TGGTCACGCTGTTCAAAAACGCCATCGTCAAGAA | |
| CGCCCACAAGAAGGGCCAGTGA | |
| 5.5.10 PORCINE (SUS SCROFFA) | |
| POMC PROTEIN (FROM SWISSPROT | |
| AAB32312) 267 AMINO ACIDS | |
| MPRLCGSRSGALLLTLLLQASMGVRGWCLESSQC | (SEQ ID NO: 10) |
| QDLSTESNLLACIRACKPDLSAETPVFPGNGDAQ | |
| PLTENPRKYVMGHFRWDRFGRRNGSSSGGGGGGG | |
| GAGQKREEEEVAAGEGPGPRGDGVAPGPRQDKRS | |
| YSMEHFRWGKPVGKKRRPVKVYPNGAEDELAEAF | |
| PLEFRRELAGAPPEPARDPEAPAEGAAARAELEY | |
| GLVAEAEAAEKKDEGPYKMEHFRWGSPPKDKRYG | |
| GFMTSEKSQTPLVTLFKNAIVKNAHKKGQ | |
| 5.5.11 BOVINE (BOS TAURUS) | |
| POMC DNA (FROM GENBANK NM_174151) | |
| ATGCCGAGACTGTGCAGCAGTCGTTCGGCCGCCC | (SEQ ID NO: 11) |
| TGCTGCTGGCCTTGCTGCTTCAGGCCTCCATGGA | |
| AGTGCGTGGTTGGTGCCTGGAGAGCAGCCAGTGT | |
| CAGGACCTCACCACGGAAAGTAACCTGCTGGCGT | |
| GCATCCGGGCCTGCAAGCCCGACCTCTCCGCCGA | |
| GACGCCGGTGTTCCCCGGCAACGGCGATGAGCAG | |
| CCGCTGACTGAGAACCCCCGGAAGTACGTCATGG | |
| GCCATTTCCGCTGGGACCGCTTCGGCCGTCGGAA | |
| TGGTAGCAGCAGCAGCGGAGTTGGGGGCGCGCCC | |
| CAGAAGCGCGAGGAGGAAGTGGCGGTGGGCGAAG | |
| GCCCCGGGCCCCGCGGCGATGACGCCGAGACGGG | |
| TCCGCGCGAGGACAAGCGTTCTTACTCCATGGAA | |
| CACTTCCCCTGGGGCAAGCCGGTGGGCAAGAAGC | |
| GGCGCCCGGTGAAGGTGTACCCCAACGGCGCCGA | |
| GGACGAGTCGGCCCAGGCCTTTCCCCTCGAATTC | |
| AAGAGGGAGCTGACCGGGGAGAGGCTCGAGCAGG | |
| CGCGCGGCCCCGAGGCCCAGGCTGAGAGTGCGGC | |
| CGCCCGGCCTGAGCTGGAGTATGGCCTGGTGGCG | |
| GAGGCGGAGGCTGAGGCGGCCGAGAAGAAGGACT | |
| CGGGGCCCTATAAGATGGAACACTTCCGCTGGGG | |
| CAGCCCGCCCAAGGACAAGCGCTACGGCGGGTTC | |
| ATGACCTCCGAGAAGAGCCAAACGCCCCTTGTCA | |
| CGCTGTTCAAAAACGCCATCATCAAGAACGCCCA | |
| CAAGAAGGGCCAGTGA | |
| 5.5.12 BOVINE | |
| (BOS TAURUS) POMC PROTEIN | |
| MPRLCSSRSAALLLALLLQASMEVRGWCLESSQC | (SEQ ID NO: 12) |
| QDLTTESNLLACIRACKPDLSAETPVFPGNGDEQ | |
| PLTENPRKYVMGHFRWDRFGRRNGSSSSGVGGAA | |
| QKREEEVAVGEGPGPRGDDAETGPREDKRSYSME | |
| HFPWGKPVGKKRRPVKVYPNGAEDESAQAFPLEF | |
| KRELTGERLEQARGPEAQAESAAARPELEYGLVA | |
| EAEAEAAEKKDSGPYKMEHFRWGSPPKDKRYGGF | |
| MTSEKSQTPLIVTLFKNAIIKNAHKKGQ | |
| 5.5.13 CANINE (CANIS FAMILIARIS) | |
| POMC DNA (FROM GENBANK AY024339) | |
| GACCTCACCACGGAAAGTAACCTGCTGGCGTGCA | (SEQ ID NO: 13) |
| TCCGGGCCTGCAAGCCCGACCTCTCCGCCGAGAC | |
| GCCCGTGCTCCCCGGCAACGGCGACGAGCAGCCG | |
| CTGGCTGAGAACCCCCGGAAGTACGTCATGGGCC | |
| ACTTCCGCTGGGACCGGTTTGGCCGCCGCAATGG | |
| CAGCGCGGGCCAGAAGCGCGAGGAAGAAGAGGTG | |
| GCGGCGGGCGGAGGCCGCGCCCCGCTGCCCGCGG | |
| GCGGCCCGGGGCCCCGCGGCGACGGTGGCGAGCT | |
| CGGCCTGCAAGAGGGCAAGCGCTCCTACTCCATG | |
| GAGCACTTCCGCTGGGGCAAGCCGGTGGGCAAGA | |
| AGCGGCGCCCGGTGAAGGTGTACCCCAACGGCGC | |
| TGAGGACGAGTCGGCCGAGGCCTTCCCCGTCGAG | |
| TTCAAGAGGGAGCTGGCCGGGCAGCGGCTGGAGC | |
| CGGCGCTCGGCCCCGAGGGCCCGGCCGCGGGCGT | |
| GGCGGCGCTGGCCGACCTGGAGTACGGCCTGGTG | |
| GCGGAGGCCGGGGCGGCCGAGAAGAAGGACGACG | |
| GGCCCTACAAGATGGAGCACTTCCGCTGGGGCAG | |
| CCCGCCCAAGGACAAGCGCTACGTCGGCTTCATG | |
| AGCTCGGAGAGGAGCCAGACGCCCCTGGTGACGC | |
| TGTTCAAAAACGCCATC | |
| 5.5.14 CANINE | |
| (CANIS FAMILIARIS) POMC | |
| PROTEIN (FROM GENBANK AAK08973; | |
| MOL ET AL., 1991) 221 AMINO ACIDS | |
| DLTTESNLIACIRACKPDLSAETPVLPGNGDEQP | (SEQ ID NO: 14) |
| LAENPRKYVMGHFRWDRFGRRNGSAGQKREEEEV | |
| AAGGGPAPLPAGGPGPRGDGGELGLQEGKRSYSM | |
| EHFRWGKPVGKKRRPVKVYPNGAEDESAEAFPVE | |
| FKRELAGQRLEPALGPEGPAAGVAALADLEYGLV | |
| AEAGAAEKKDDGPYKMEHFRWGSPPKDKRYVGFM | |
| SSERSQTPLVTLFKNAI | |
| 5.5.15 | |
| GUINEA PIG (CAVIA PORCELLUS) | |
| POMC DNA (FROM GENBANK S78260) | |
| ATGCCGAGATCGTGCTACAGCCGCTCGGGGACCC | (SEQ ID NO: 15) |
| TGCTGCTGGCCTTGCTGCTTCAGATCTCCATGGA | |
| AGTGCGGGGCTGGTGCCTGGAGAGCAGCCAGTGT | |
| CAGGACCTCACCACGGAGAGACACCTGCTGGAGT | |
| GCCTCCGGGCCTGCAAACCGGACCTCTCGGCCGA | |
| GACGCCAGTGTTTCCGGGGGGCGCCGACGAGCAG | |
| ACGCCGACCGAGAGCCCCCGGAAGTACGTCACGG | |
| GCCACTTCCGCTGGGGCCGCTTCGGCCGCGGTAA | |
| CAGCAGCGGCGCGAGCCAGAAGCGTGAGGAGGAG | |
| GCGGCGGCGGCCGACCCCGGCTTCCACGGCGATG | |
| GCGTCGAGCCGGGCCTGCGCGAGGACAAGCGCTC | |
| CTACTCCATGGAGCACTTCCGCTGGGGCAAGCCG | |
| GTGGGCAAGAAGCGGCGCCCGGTGAAGGTGTACG | |
| CGAACGGCGCGGAGGAGGAGTCGGCCGAGGCCTT | |
| TCCGCTTGAGTTCAAGCGGGAGCTGACCGGGGAG | |
| CGGCCCGCGGCGGCGCCCGGCCCCGACGGCCTGG | |
| GGTTCGGCCTGGTGGCTGAGGCCGAGGCCGAGGC | |
| GGCAGCGGCCGAGAAGAAGGACGCGGCCGAGAAG | |
| AAGGACGACGGGTCCTATCGCATGGAGCACTTCC | |
| GCTGGGGCACCCCGCGCAAGGGCAAGCGCTACGG | |
| CGGCTTCATGACCTCGGAGAAGAGCCAGACGCCG | |
| CTGGTGACGCTGTTCAAGAACGCCATCGTCAAGA | |
| ACGCCCACAAGAAGGGCCAGTGA | |
| 5.5.16 | |
| GUINEA PIG (CAVIA PORCELLUS) | |
| POMC PROTEIN (FROM GENBANK | |
| A54322; SMITH ET AL., 1987) | |
| MPRSCYSRSGTLLLALLLQISMEVRGWCLESSQC | (SEQ ID NO: 16) |
| QDLTTERHLLECLRACKPDLSAETPVFPGGADEQ | |
| TPTESPRKYVTGHFRWGRFGRGNSSGASQKREEE | |
| AAAADPGFHGDGVEPGLREDKRSYSMEHFRWGKP | |
| VGKKRRPVKVYANGAEEESAEAFPLEFKRELTGE | |
| RPAAAPGPDGLGFGLVAEAEAEAAAAEKKDAAEK | |
| KDDGSYRMEHFRWGTPRKGKRYGGFMTSEKSQTP | |
| LVTLFKNAIVKNAHKKGQ | |
| 5.5.17 RAT (RATTUS NORVEGICUS) | |
| POMC DNA (FROM GENBANK AH002232) | |
| ATGCCGAGATTCTGCTACAGTCGCTCAGGGGCCC | (SEQ ID NO: 17) |
| TGCTGCTGGCCCTCCTGCTTCAGACCTCCATAGA | |
| CGTGTGGAGCTGGTGCCTGGAGAGCAGCCAGTGC | |
| CAGGACCTCACCACGGAAAGCAACCTGCTGGCTT | |
| GCATCCGGGCCTGCAGACTCGACCTCTCGGCGGA | |
| GACGCCCGTGTTTCCAGGCAACGGAGATGAACAG | |
| CCCTTGACTGAAAATCCCCGGAAGTACGTCATGG | |
| GTCACTTCCGCTGGGACCGCTTCGGCCCGAGAAA | |
| CAGCAGCAGTGCTGGCGGCTCAGCGCAGAGGCGT | |
| GCGGAGGAAGAGACGGCGGGGGGAGATGGCCGTC | |
| CGGAGCCAAGTCCACGGGAGGGCAAGCGCTCCTA | |
| CTCCATGGAGCACTTCCGCTGGGGCAAGCCGGTG | |
| GGCAAGAAGCGGCGCCCTGTGAAGGTGTACCCCA | |
| ATGTCGCCGAGAACGAGTCGGCCGAGGCCTTTCC | |
| CCTAGAGTTCAAGAGGGAGCTGGAAGGCGAGCAG | |
| CCTGATGGCTTGGAGCACGTCCTGGAGCCGGATA | |
| CCGAGAAGGCCGACGGGCCCTATCGGGTGGAGCA | |
| CTTCCGCTGGGGCAACCCGCCCAAGGACAAGCGC | |
| TACGGCGGCTTCATGACCTCCGAGAAGAGCCAGA | |
| CGCCCCTGGTGACGCTCTTCAAGAACGCCATCAT | |
| CAAGAACGCGCACAAGAAGGGCCAGTGAGGGTGC | |
| 5.5.18 RAT (RATTUS | |
| NORVEGICUS) POMC PROTEIN (FROM | |
| GENBANK NP_647542) 235 AMINO ACIDS | |
| MPRFCYSRSGALLLALLLQTSIDVWSWCLESSQC | (SEQ ID NO: 18) |
| QDLTTESNLLACIPACRLDLSAETPVFPGNGDEQ | |
| PLTENPRKYVMGHFRWDRFGPRNSSSAGGSAQRR | |
| AEEETAGGDGRPEPSPREGKRSYSMEHFRWGKPV | |
| GKKRRPVKVYPNVAENESAEAFPLEFKRELEGEQ | |
| PDGLEQVLEPDTEKADGPYRVEHFRWGNPPKDKR | |
| YGGFMTSEKSQTPLVTLFKNAIIKNVHKKGQ | |
| 5.5.19 MURINE (MUS MUSCULUS) | |
| POMC DNA (FROM GENBANK AH005319) | |
| ATGCCGAGATTCTGCTACAGTCGCTCAGGGGCCC | (SEQ ID NO: 19) |
| TGTTGCTGGCCCTCCTGCTTCAGACCTCCATAGA | |
| TGTGTGGAGCTGGTGCCTGGAGAGCAGCCAGTGC | |
| CAGGACCTCACCACGGAGAGCAACCTGCTGGCTT | |
| GCATCCGGGCTTGCAAACTCGACCTCTCGCTGGA | |
| GACGCCCGTGTTTCCTGGCAACGGAGATGAACAG | |
| CCCCTGACTGAAAACCCCCGGAAGTACGTCATGG | |
| GTCACTTCCGCTGGGACCGCTTCGGCCCCAGGAA | |
| CAGCAGCAGTGCTGGCAGCGCGGCGCAGAGGCGT | |
| GCGGAGGAAGAGGCGGTGTGGGGAGATGGCAGTC | |
| CAGAGCCGAGTCCACGCGAGGGCAAGCGCTCCTA | |
| CTCCATGGAGCACTTCCGCTGGGGCAAGCCGGTG | |
| GGCAAGAAACGGCGCCCGGTGAAGGTGTACCCCA | |
| ACGTTGCTGAGAACGAGTCGGCGGAGGCCTTTCC | |
| CCTAGAGTTCAAGAGGGAGCTGGAAGGCGAGCGG | |
| CCATTAGGCTTGGAGCAGGTCCTGGAGTCCGACG | |
| CGGAGAAGGACGACGGGCCCTACCGGGTGGAGCA | |
| CTTCCGCTGGAGCAACCCGCCCAAGGACAAGCGT | |
| TACGGTGGCTTCATGACCTCCGAGAAGAGCCAGA | |
| CGCCCCTGGTGACGCTCTTCAAGAACGCCATCAT | |
| CAAGAACGCGCACAAGAAGGGCCAGTGA | |
| 5.5.20 MURINE | |
| (MUS MUSCULUS) POMC PROTEIN | |
| MPRFCYSRSGALLLALLLQTSTDVWSWCLESSQC | (SEQ ID NO: 20) |
| QDLTTESNLLACIRACKLDLSLETPVFPGNGDEQ | |
| PLTENPRKYVMGHFRWDRFGPRNSSSAGSAAQRR | |
| AEEEAVWGDGSPEPSPREGKRSYSMEHFRWGKPV | |
| GKKRRPVKVYPNVAENESAEAFPLEFKRELEGER | |
| PLGLEQVLESDAEKDDGPYRVEHFRWSNPPKDKR | |
| YGGFMTSEKSQTPLVTLFKNAIIKNAHKKGQ | |
| 5.5.21 CHICKEN (GALLUS | |
| GALLUS) POMC DNA (FROM GENBANK | |
| A54322; SMITH ET AL., 1987) | |
| ATGCGGGGCGCGCTGTGCCACAGCCTGCCCGTGG | (SEQ ID NO: 21) |
| TGCTGGGGCTGCTGCTGTGTCACCCCACCACCGC | |
| CAGCGGCCCATGCTGGGAGAACAGCAAGTGCCAG | |
| GACCTGGCCACCGAGGCTGGTGTTTTGCAGGCGT | |
| GTGCCAAGGCATGCCGTGCTGAGCTGTCGGCCGA | |
| GGCACCCGTGTACCCGGGCAATGGGCACCTGCAG | |
| CCCCTCTCGGAGAGCATCCGCAAGTACGTGATGA | |
| GCCATTTCCGCTGGAACAAGTTCGGCCGTCGCAA | |
| CAGCAGCAGCGGAGGGCACAAAAGGGAGGAGGTG | |
| GCCGGCCTCGCCCTGCCTGCCGCGTCACCCCACC | |
| ACCCCGCCGGGGAGGAGGAAGATGGAGAAGGGTT | |
| GGAACGAGAGGAAGGGAAGCGCTCCTACTCCATG | |
| GAGCATTTCCGCTGGGGCAAGCCGGTGGGGCGGA | |
| AGAGGAGACCCATCAAGGTGTACCCCAACGGGGT | |
| GGACGAGGAGTCGGCTGAGAGTTACCCCATGGAG | |
| TTCCGGAGGGAGATGGCGCCCGATGGGGACCCCT | |
| TCGGCCTCTCCGAGGAGGAGGAAGAAGAGGAGGA | |
| AGAGGAAGGCGAGGAGGAAAAGAAGGATGGAGGC | |
| TCGTACCGCATGCGGCACTTCCGCTGGCACGCGC | |
| CGCTGAAGGACAAGCGCTACGGCGGCTTCATGAG | |
| CTTGGAGCACAGCCAGACCCCGCTGATGACTCTG | |
| TTCAAAAACGCCATCGTCAAAAGCGCCTACAAGA | |
| AGGGTCAGTGA | |
| 5.5.22 CHICKEN | |
| (GALLUS GALLUS) POMC PROTEIN | |
| (FROM GENBANK BAA34366; TAKEUCHI | |
| ET AL., 1999) 251 AMINO ACIDS | |
| MRGALCHSLPVVLGLLLCHPTTASGPCWENSKCQ | (SEQ ID NO: 22) |
| DLATEAGVLACAKACPAELSAEAPVYPGNGHLQP | |
| LSESIRKYVMSHFRWNKFGRRNSSSGGHKREEVA | |
| GLALPAASPHHPAGEEEDGEGLEREEGKRSYSME | |
| HFRWGKPVGRKRRPIKVYPNGVDEESAESYPMEF | |
| RREMAPDGDPFGLSEEEEEEEEEEGEEEKKDGCS | |
| YRMRHFRWHAPLKDKRYGGFMSLEHSQTPLMTLF | |
| KNAIVKSAYKKGQ | |
| 5.5.23 GIANT TOAD POMC DNA | |
| ATGTTGCAGCCAGGGTGGAGATGTATCCTGACAA | (SEQ ID NO: 23) |
| TACTCGGGGCGTTTATATTTCATGTCGGTGAGGT | |
| CAAGAGTCAGTGCTGGGAGAGCGGTAAATGTGCA | |
| GATCTGACGAGCGAGGATGGGATACTGGAATGTA | |
| TTAAAGACTGCAAGATGGTCCTGTCTGCAGAGTC | |
| ACCAGTGTTTCCTGGGAATGGACACATGCAACCC | |
| CTCTCTGAAAACATCAGGAAGTATGTCATGAGCC | |
| ACTTCCGCTGGAATAAGTTTGGCCGAAGGAATAG | |
| CACCGGTGGCGATAGCAACAACGCAGGTTACAAA | |
| CGGGAAGATATAGCCAACTACCCCATATTTAACC | |
| TGTTCCCCACTAATGACAACCAAAACACACAAGA | |
| TGGCAACATGGAAGAAGAACTACGCAGGCAAGAC | |
| AACAAGAGGTCATATTCTATGGAACACTTCCGAT | |
| GGGGTAAACCAGTCGGGAAAAAAAGGAGACCTAT | |
| TAAGGTTTTCCCAAGCGATGCTGAAGAAGAATCA | |
| TCTGAAATCTSCCCAACAGAGTACAGAAGAGAGT | |
| TGTCTGTAGAGTTTGACTACCCCGATACCAACTC | |
| TGAAGAAGACATGGACGACAGCATGTTGATGGAA | |
| AGCCCAAATAGAAAAGATCGGAAGTATAAAATGC | |
| ATCATTTTCGATGGGAAGGTCCACCCAAAGACAA | |
| AAGATATGGAGGATTCATGACCCCTGAGCGCAGT | |
| CAGACTCCACTAATGACTCTTTTCAAAAATGCCA | |
| TTATCAAAAATGCCCACAAGAAGGGTCAATAA | |
| 5.5.24 GIANT TOAD | |
| (BUFO MARINUS) POMC PROTEIN | |
| (FROM GENBANK AAF06345; ALRUBAIAN | |
| ET AL., 1999) 259 AMINO ACIDS | |
| MLQPGWRCILTILGAFTFHVGEVKSQCWESGKCA | (SEQ ID NO: 24) |
| DLTSEDGILECIKDCKMVLSAESPVFPGNGHMQP | |
| LSENIRKYVMSHFRWNKFGRRNSTGGDSNNAGYK | |
| REDIANYPIFNLFPTNDNQNTQDGNMEEELRRQD | |
| NKRSYSMEHFRWGKPVGKKRRPIKVFPSDAEEES | |
| SEIXPTEYRRELSVEFDYPDTNSEEDMDDSMLME | |
| SPNRKDRKYKMHHFRWEGPPKDKRYGGFMTPERS | |
| QTPLMTLFKNAIIKNAHKKGQ | |
| 5.5.25 CARP POMC DNA | |
| ATGGTGAGGGGAGAGAGGATGTTGTGTCCTGCTT | (SEQ ID NO: 25) |
| GGCTCTTGGCTCTGGCTGTTCTGTGTGCGGCTGG | |
| ATCTGAAGTCAGAGCTCAGTGTATGGAGGACGCC | |
| CGCTGCAGAGACCTCACCACTGATGAGAACATCT | |
| TGGACTGCATACAGCTATGCAGGTCTGATCTGAC | |
| AGATGAAACCCCCGTCTACCCTGGAGAAAGCCAT | |
| TTGCAGCCTCCCTCTGAGCTGGAGCAAACCGAGG | |
| TCCTCGTACCCCTGTCCCCAGCGGCCCTCGCTCC | |
| TGCTGAGCAAATGGACCCCGAGTCCAGCCCTCAG | |
| CACGAGCACAAGCGCTCCTACTCCATGGAGCATT | |
| TCCGCTGGGGAAAGCCAGTGGGTCGCAAGCGCAG | |
| GCCTATCAAGGTGTACACCAACGGCGTGGAGGAG | |
| GAATCCACCGAGACTCTCCCAGCTGAGATGAGGC | |
| GCGAGCTGGCTACAAACGAGATCGACTATCCTCA | |
| AGAGGAGGGCGCTTTAAACCAGCAGGATAAGAAG | |
| GATGGCTCCTACAAAATGAGCCATTTCCGCTGGA | |
| GCAGCCCGCCTGCTAGCAAGCGCTATGGAGGCTT | |
| CATGAAGTCCTGGGACGAGCGCAGTCAGAAACCC | |
| CTTCTCACGCTCTTCAAAAACGTCATAAACAAAG | |
| AGCACCAGAAGAAGGACCAGTGA | |
| 5.5.26 CARP POMC PROTEIN | |
| (FROM GENBANK Y14618) | |
| MVRGERMLCPAWLLALAVLCAAGSEVPAQCMEDA | (SEQ ID NO: 26) |
| RCRDLTTDENILDCIQLCRSDLTDETPVYPGESH | |
| LQPPSELEQTEVLVPLSPAALAPAEQMDPESSPQ | |
| HEHKRSYSMEHFRWGKPVGRKRRPIKVYTNGVEE | |
| ESTETLPAEMRRELATNEIDYPQEEGALNQQDKK | |
| DGSYKMSHFRWSSPPASKRYGGFMKSWDERSQKP | |
| LLTLFKNVINKEHQKKDQ | |
| 5.5.27 DANIO POMC DNA | |
| (FROM GENBANK AY125332) | |
| ATGGTGAGGGGAGTGAGGATGTTGTGTCCTGCTT | (SEQ ID NO: 27) |
| GGCTCTTGGCTCTGGCTGTTCTCTGCGCAGGAGG | |
| ATCTGAAGTCAGAGCTCAGTGTTGGGAAAATGCC | |
| CGCTGTCGAGACCTCAGCACAGAGGAGAACATCT | |
| TGGAATGCATACAATTATGCAGGTCTGAACTTAC | |
| AGATGAAACCCCCGTCTACCCTGGAGAAAGCCAT | |
| CTACAGCCTCCCTCCGAGCCGGAGCAAATCGACC | |
| TCCTCGCACACCTTTCCCCTGTAGCACTCGCAGC | |
| CCCTGAACAGATAGAGCCGGAGTCCGGCCCTCGA | |
| CACGACCACAAGCGCTCCTACTCCATGGAACACT | |
| TCCGGTGGGGCAAACCGGTCGGCCGCAAACGCAG | |
| ACCCATCAAGGTGTACACGAACGGCGTGGAAGAG | |
| GAATCCGCCGAAACGCTTCCGGAAGAGATGAGAC | |
| GCGAGCTGGCAAATAACGAGGTCGACTATCCGCA | |
| AGAAGAGATGCCTTTAAACCCACTGGGAAAGAAG | |
| GACCCCCCCTACAAAATGACCCATTTCCGCTGGA | |
| GCGTCCCGCCGGCTAGCAAGCGCTATGGAGGCTT | |
| CATGAAGTCCTGGGACGAGCGTGCTCAGAAACCA | |
| CTGCTCACACTCTTCAAAAACGTAATGCATAAAG | |
| GCCAACCGAGGAAGGATGAGTGA | |
| 5.5.28 DANIO POMC PROTEIN | |
| (FROM GENBANK AY135148) | |
| MVRGVRMLCPAWLLALAVLCAGGSEVRAQCWENA | (SEQ ID NO: 28) |
| RCRDLSTEENILEGIQLCRSELTDETPVYPGESH | |
| LQPPSEPEQIDLLAHLSPVALAAPEQIEPESGPR | |
| HDHKRSYSMEHFRWGKPVGRKRRPIKVYTNGVEE | |
| ESAETLPEEMRRELANNEVDYPQEEMPLNPLGKK | |
| DPPYKMTHFRWSVPPASKRYGGFMKSWDERAQKP | |
| LLTLFKNVMHKGQPRKDE | |
| 5.5.29 BULLFROG POMC DNA | |
| (FROM GENBANK X15510) | |
| ATGTTGCAGCCAGTCTGGCACGCCTGTATCCTGG | (SEQ ID NO: 29) |
| CAATACTTGGGGTGTTCATATTTCATGTCGGAGA | |
| GGTCCGGAGCCAGTGCTGGGAAAGCAATAAGTGT | |
| ACAGATTTAAGCAGCGAAGATGGCATTCTGGAAT | |
| GTATCAAAGCATGCAAGATGGACCTCTCTGCAGA | |
| ATCTCCCGTGTTTCCCGGCAATGGCCACATCCAG | |
| CCCCTTTCTGAAAACATCAGGAAATATGTCATGA | |
| GCCACTTTCGCTGGAATAAATTTGGTAGAAGGAA | |
| CAGCACCAGCAATGACAACAACAACAACAATGGT | |
| GGCTATAAGCGGGAGGATATTGCCAACTACCCTA | |
| TATTGAACCTGTTCCTTGGCAGCGACAACCAAAA | |
| CACACAGGAGGGAATTATGGAAGATGACGCCTTG | |
| GATAGGCAAGACAGCAAAAGGTCTTATTCCATGG | |
| AGCACTTCCGATGGGGAAAACCCGTCGGCAAGAA | |
| GAGGAGGCCTATCAAAGTTTTCCCCACAGATGCT | |
| GAAGAAGAGTCCTCAGAAAGTTTCCCCATTGAGC | |
| TGAGAAGAGAGCTCTCTCTAGAGTTTGACTATCC | |
| TGACACCAACTCCGAAGAAGAATTGGATAATGGC | |
| GAGCTGCTAGAAGGTCCAGTTAAAAAAGGTAGGA | |
| AGTACAAAATGCACCATTTCCGATGGGAAGGACC | |
| TCCCAAAGACAAGCGGTATGGTGGATTTATGACC | |
| CCAGAGAGAAGCCAGACACCTTTAATGACTCTTT | |
| TCAAGAATGCTATAATTAAGAACGCCCACAAAAA | |
| GGGCCAGTAG | |
| 5.5.30 BULLFROG POMC PROTEIN | |
| MLQPVWHACILAILGVFIFHVGEVRSQCWESNKC | (SEQ ID NO: 30) |
| TDLSSEDGILECIKACKMDLSAESPVFPGNGHIQ | |
| PLSENIRKYVMSHFRWNKFGRRNSTSNDNNNNNG | |
| GYKREDIANYPILNLFLGSDNQNTQEGIMEDDAL | |
| DRQDSKRSYSMEHFRWGKPVGKKRRPIKVFPTDA | |
| EEESSESFPIELRRELSLEFDYPDTNSEEELDNG | |
| ELLEGPVKKGRKYKMHHFRWEGPPKDKRYGGFMT | |
| PERSQTPLMTLFKNAIIKNAHKKGQ | |
| 5.5.31 OPOSSUM (MONODELPHIS | |
| DOMESTICA) POMC PROTEIN (FROM | |
| GENBANK AAL13338; KACSOH ET AL.) | |
| MPKPSWSYLGALLVAVLFQASVEVHGWCLQASNC | (SEQ ID NO: 31) |
| RDSKAEDGLVECIKSCKMDLSAESPVFPGNGQYE | |
| PLSENIRKYVMSHFRWNKFGRRNISSGSISSDGG | |
| NVGQKRQELMQGDFLDLPPPGVWGEDEEMQEGLP | |
| LIRKARELQNKRSYSMEHFRWGKPVGKKRRPVKI | |
| YPNGVEEESAESYPVEIRRDLPMKINFPEYPELA | |
| IDEEEAAKEVYEEKVKKDGGGYKMEHFRWGTPPK | |
| DKRYGGFMISEKSHTPLMTLFKNAIIKNGHKKGQ | |
| 5.5.32 SHEEP (OVIS ARIES) POMC | |
| PROTEIN (FROM SWISSPROT CAD45184) | |
| MPRLCSSRSGALLLVLLLQASMEVRGWCLESSQC | (SEQ ID NO: 32) |
| QDLTTESNLLACIRACKPDLSAETPVFPGNGDEQ | |
| PLTENPRKYVMGHFRWDRFGRRNGSSSFGAGGAA | |
| QKREEEVAVGEGPGPRGDGAETGPREDKRSYSME | |
| HFRWGKPVGKKRRPVKVYPNGAEDESAQAFPLEF | |
| KRELTGERLEQARXPEAQAESAAARAELEYGLVA | |
| EAEAAEKKDSGPYKMEHFRWGSPPKDKRYGGFMT | |
| SEKSQTPLVTLFKMAIIKNAHKKGQ | |
| 5.5.33 SHEEP (OVIS ARIES) PARTIAL | |
| POMC PROTEIN (FROM GENBANK P01191 | |
| LEVIN ET AL, 1993) 212 AMINO ACIDS | |
| PDLSAETPVFPGNCDEQPLTENPRKYVMGHFRWD | (SEQ ID NO: 33) |
| RFGRRNGSSSFGAGGAAQKREEEVAVGEGPGPRG | |
| DGAETGPREDKRSYSMEHFRWGKPVGKKRRPVKV | |
| YPNGAEDESAQAFPLEFXXELTGERLEQARGPEA | |
| QAESAAARAELEYGLVAEAEAAEKKDSGPYKMEH | |
| FRWGSPPKDKRYGGFMTSEKSQTPLVTLFKNAII | |
| KNAHKKGQ | |
| 5.5.34 GIANT | |
| BULLFROG (RANA CATESBEIANA) POMC | |
| PROTEIN (FROM GENBANK P11885; PAN | |
| AND CHANG, 1989) 263 AMINO ACIDS | |
| MLQPVWHACILAILGVFIFHVGEVRSQCWESNKC | (SEQ ID NO: 34) |
| TDLSSEDGILECIKACKMDLSAESPVFPGNGHIQ | |
| PLSENIRKYVMSHFRWNKFGRRNSTSNDNNNNNG | |
| GYKREDIANYPILNLFLGSDNQNTQEGIMEDDAL | |
| DRQDSKRSYSMEHFRWGKPVGKKRRPIKVFPTDA | |
| EEESSESFPIELRRELSLEFDYPDTNSEEELDNG | |
| ELLEGPVKKGRKYKMHHFRWEGPPKDKRYGGFMT | |
| PERSQTPLMTLFKNAIIKNAHKKGQ | |
| 5.5.35 MEXICAN SPADEFOOT | |
| TOAD (SPEA MULTIPLICATA) POMC | |
| PROTEIN (FROM GENBANK AAD21040; | |
| LEE ET AL., 1999) 258 AMINO ACIDS | |
| MLCPVWSCLFAVLGVFVFHVGEVRGQCWQSAKCM | (SEQ ID NO: 35) |
| DLESEDGILECIKACKTDLSAESPIFPGNGHLQP | |
| LAENVRKYVMSHFRWNKFGRRNTTGNEGNSGSKR | |
| EDIANYPIFNLFPSSNGQNTEDNNWKKYQDRQDN | |
| KRSYSMEHFRWGKPVGRKRRPIKVFPNGMEEESS | |
| ESYPMELRRELSLEDDYPEIDSEDDLDYNDLLSM | |
| PKFKGGDYRIHHFRWGSPPKDKRYGGFMTPERSQ | |
| TPLMTLFKNAIIKNAHKKAQ | |
| 5.5.36 AFRICAN CLAWED | |
| FROG (XENOPUS LAEVIS) POMC | |
| PROTEIN (FROM SWISSPROT P06298; | |
| MARTENS, 1986) 259 AMINO ACIDS | |
| MFRPLWGCFLAILGICIFHIGEVQSQCWESSRCA | (SEQ ID NO: 36) |
| DLSSEDGVLECIKACKTDLSAESPVFPGNGHLQP | |
| LSESIRKYVMTHFRWNKFGRRNSTGNDGSNTGYK | |
| REDISSYPVFSLFPLSDQNAPGDNMEEEPLDRQE | |
| NKRAYSMEHFRWGKPVGRKRRPIKVYPNGVEEES | |
| AESYPMELRRELSLELDYPEIDLDEDIEDNEVKS | |
| ALTKKNGNYRMHHFRWGSPPKDKRYGGFMTPERS | |
| QTPLMTLFKNAIIKNSHKKGQ | |
| 5.5.37 MUDPUPPY | |
| (NECTURUS MACULOSUS) POMC PROTEIN | |
| (FROM GENBANK AAN46359; KOZAK | |
| ET AL., 1986) 262 AMINO ACIDS | |
| MLKPVWSCLFATLGALLCQTVVAHSQCWESSKCR | (SEQ ID NO: 37) |
| DLATEGSVLECIKACKVELSAESPVYPGNGHMQP | |
| LSENIRKYVMSHFRWNQFGRKNSTVASGNGAGSK | |
| REELSGNPIISLFTTSESQSSGAHDSXKEGEVMD | |
| RQDNKRSYSMEHFRWGKPVGRKRRPIKVYPNGVE | |
| EESSESYPLELKRDLSLGLEYPEFDSQEGLENNE | |
| VMVVLPEKKDGNYRMHHFRWGSPPKDKRYGGFMT | |
| PERSQTPLMTLFKNAIKNAHKKGQ | |
| 5.5.38 TWO-TOED AMPHIUMA | |
| (AMPHIUMA MEANS) POMC PROTEIN | |
| (FROM GENBANK AAN46358; KOZAK | |
| ET AL., 1986) 262 AMINO ACIDS | |
| MLRPVWSCLPATLGALLCQTAGANSQCWESSKCR | (SEQ ID NO: 38) |
| DLATEGSVLECIKACKVELSAESPVYPGNGHMQP | |
| LSENIRKYVMSHFRWNKFGRKNSTSVSGNSAGNK | |
| REELSNNPIISLFTTSESQSSGADDGNKEGEAME | |
| RQDSKRSYSMEHFRWGKPVGRKRRPIKVYPNGVE | |
| EESSESYPLELRRDLSLGLDYPDSDSQEGLENNE | |
| ITTGLTKKNDKQYRIGHFRWGSPLKDKRYGGFMT | |
| PERSQTPLMTLFKNAIKNAHKKGQ | |
| 5.5.39 CHINESE SOFTSHELL | |
| TURTLE (PELODISCUS SINENSIS) POMC | |
| PROTEIN (FROM GENBANK AAM34798; | |
| SHEN ET AL., 2002) 261 AMINO ACIDS | |
| MLKPVRSGLLAILGVLLFHADGGVHSQCWDSSRC | (SEQ ID NO: 39) |
| RELSTDAGLLECIKACKMDLSDESPMYPGNGHLQ | |
| PLSENIRKYVMSHFRWNKFGRKNSSSSVAGHKRE | |
| EIPSHLLLGLFPDVAPAQRGDDGEGGAALERQDS | |
| KRSYSMEHFRWGKPVGRKRRPIKVYPSEVEEESA | |
| ESYPPEFRRDLSMELDYPEFESLEDPESEEALVS | |
| EEAEKKDGNSYKMHHFRWNAPPKDKRYGGFMTSE | |
| SSQTPLMTLFKNAIIKNAYKKGQ | |
| 5.5.40 CHIMPANZEE (PAN | |
| TROGLODYTES) POMC PROTEIN (FROM | |
| GENBANK AAM76608; O'HUIGIN ET AL.) | |
| SAETPMFPGNGDEQPLTENPRKYVMGHFRWDRFG | (SEQ ID NO: 40) |
| RRNSSSSSSGSGAGQKREDVSAGEDRGPLPEGGP | |
| EPRSDGAKPGPREGKRSYSMEHFRWGKPVGKKRR | |
| PVKVYPNGAEDESAEAFPLEFKRELTGQRPREGD | |
| GPDGPADDGAGAQADLEHSLLVAAEKKDEGPYRM | |
| EHFRWGSPPKDKRYGGF | |

### 6.0 REFERENCES

The following references, to the extent that they provide exemplary procedural or other details supplementary to those set forth herein, are specifically incorporated herein by reference.
U.S. Pat. No. 4,237,224, issued Dec. 2, 1980.
U.S. Pat. No. 4,554,101, issued Nov. 19, 1985.
U.S. Pat. No. 4,683,195, issued Jul. 28, 1987.
U.S. Pat. No. 4,683,202, issued Jul. 28, 1987.
U.S. Pat. No. 4,800,159, issued Jan. 24, 1989.
U.S. Pat. No. 4,883,750, issued Nov. 28, 1989.
U.S. Pat. No. 4,987,071.
U.S. Pat. No. 5,037,746.
U.S. Pat. No. 5,093,246.
U.S. Pat. No. 5,116,742.
U.S. Pat. No. 5,145,684, issued Sep. 8, 1992.
U.S. Pat. No. 5,334,711.
U.S. Pat. No. 5,354,855.
U.S. Pat. No. 5,399,363, issued Mar. 21, 1995.
U.S. Pat. No. 5,466,468, issued Nov. 14, 1995.
U.S. Pat. No. 5,543,158, issued Apr. 6, 1996.
U.S. Pat. No. 5,552,157, issued Sep. 3, 1996.
U.S. Pat. No. 5,565,213, issued Oct. 15, 1996.
U.S. Pat. No. 5,567,434, issued Oct. 22, 1996.
U.S. Pat. No. 5,602,306, issued Feb. 11, 1997.
U.S. Pat. No. 5,631,359.
U.S. Pat. No. 5,639,655.
U.S. Pat. No. 5,639,940, issued Jun. 17, 1997.
U.S. Pat. No. 5,641,515, issued Jun. 24, 1997.
U.S. Pat. No. 5,646,020.
U.S. Pat. No. 5,646,031.
U.S. Pat. No. 5,656,016.
U.S. Pat. No. 5,697,899.
U.S. Pat. No. 5,720,936, issued Feb. 24, 1998.
U.S. Pat. No. 5,738,868, issued Apr. 14, 1998.
U.S. Pat. No. 5,741,516, issued Apr. 21, 1998.
U.S. Pat. No. 5,770,219.
U.S. Pat. No. 5,779,708.
U.S. Pat. No. 5,783,208.
U.S. Pat. No. 5,789,655, issued Aug. 4, 1998.
U.S. Pat. No. 5,795,587, issued Aug. 18, 1998.
U.S. Pat. No. 5,797,898.
U.S. Pat. No. 5,849,902.
Int. Pat. Appl. No. PCT/US87/00880.
Int. Pat. Appl. No. PCT/US89/01025.
Int. Pat. Appl. Publ. No. WO 88/10315.
Int. Pat. Appl. Publ. No. WO 89/06700.
Int. Pat. Appl. Publ. No. WO 91/03162.
Int. Pat. Appl. Publ. No. WO 92/07065.
Int. Pat. Appl. Publ. No. WO 93/15187.
Int. Pat. Appl. Publ. No. WO 93/23569.
Int. Pat. Appl. Publ. No. WO 94/02595.
Int. Pat. Appl. Pubi. No. WO 94/13688.
Int. Pat. Appl. Publ. No. WO 98/13526.
Eur. Pat. Appl. Publ. No. EP 0320308.
Eur. Pat. Appl. Publ. No. EP 0329822.
Eur. Pat. Appl. Publ. No. EP 0360257.
Eur. Pat. Appl. Pubi. No. 92110298.4.
Great Britain Appl. No. 2202328.
Afione, Conrad, Kearns, Chunduru, Adams, Reynolds, Guggino, Cutting, Carter and Flotte, “In vivo model of adeno-associated virus vector persistence and rescue,” J. Virol., 70:3235-41, 1996.
Allen and Choun, “Large unilamellar liposomes with low uptake into the reticuloendothelial system,” FEBS Lett., 223:42-46, 1987.
Angel, Bauman, Stein, Dellus, Rahmsdorf and Herrlich, “12-0-tetradecanoyl-phorbol-13-acetate induction of the human collagenase gene is mediated by an inducible enhancer element located in the 5′ flanking region,” Mol. Cell. Biol., 7:2256, 1987a.
Angel, Imagawa, Chiu, Stein, Imbra, Rahmsdorf, Jonat, Herrlich and Karin, “Phorbol ester-inducible genes contain a common cis element recognized by a TPA-modulated trans-acting factor,” Cell, 49:729, 1987b.
Appleyard, Hayward, Young, Butler, Cone, Rubinstein and Low, “A role for the endogenous opioid beta-endorphin in energy homeostasis,” Endocrinology, 144:1753-60, 2003.
Atchison and Perry, “Tandem kappa immunoglobulin promoters are equally active in the presence of the kappa enhancer: Implications for model of enhancer function,” Cell, 46:253, 1986.
Atchison and Perry, “The role of the kappa enhancer and its binding factor NF-kappa B in the developmental regulation of kappa gene transcription,” Cell, 48:121, 1987.
Balazsovits et al., “Analysis of the effect of liposome encapsulation on the vesicant properties, acute and cardiac toxicities, and antitumor efficacy of doxorubicin,” Cancer Chemother. Pharmacol., 23:81-86, 1989.
Banerji, Olson and Schaffner, “A lymphocyte-specific cellular enhancer is located downstream of the joining region in immunoglobulin heavy-chain genes,” Cell, 35:729, 1983.
Banerji, Rusconi and Schaffner, “Expression of a β-globin gene is enhanced by remote SV40 DNA sequences,” Cell, 27:299, 1981.
Bartlett and Samulski, “Fluorescent viral vectors: a new technique for the pharmacological analysis of gene therapy,” Nat. Med.,4(5):635-7, 1998.
Baskin, Hahn and Schwartz, “Leptin sensitive neurons in the hypothalamus,” Horm. Metab. Res., 31:345-50, 1999.
Beck, Jones, Chesnut, Walsh, Reynolds, Carter, Askin, Flotte and Guggino, “Repeated delivery of adeno-associated virus vectors to the rabbit airway,” J. Virol., 73:9446-55, 1999.
Bergen, Mizuno, Taylor and Mobbs, “Hyperphagia and weight gain after gold-thioglucose: relation to hypothalamic neuropeptide Y and proopiomelanocortin,” Endocrinology, 139:4483-88, 1998.
Berkhout, Silverman and Jeang, “tat trans-activates the human immunodeficiency virus through a nascent RNA target,” Cell, 59:273, 1989.
Berns, In FIELDS VIROLOGY, Fields, (ed.), Raven Press, Philadelphia, Pa., pp. 2173-97, 1996.
Berns and Linden, “The cryptic life style of adeno-associated virus,yn” Bioessays, 17:237-45, 1995.
Berns et al., In: Virus Persistence, Mehay et al. (Ed.), Cambridge Univ. Press, pp. 249-265, 1982.
Berns, Pinkerton, Thomas, Hoggan, “Detection of adeno-associated virus (AAV)-specific nucleotide sequences in DNA isolated from latently infected Detroit 6 cells,” Virol., 68(2):556-560, 1975.
Blanar, Baldwin, Flavell and Sharp, “A gamma-interferon-induced factor that binds the interferon response sequence of the MHC Class I gene, H-2Kb,” EMBO J., 8:1139, 1989.
Bodine and Ley, “An enhancer element lies 3′ to the Human A gamma globin gene,” EMBO J., 6:2997, 1987.
Boshart, Weber, Jahn, Dorsch-Hasler, Fleckenstein and Schaffner, “A very strong enhancer is located upstream of an immediate early gene of human cytomegalovirus,” Cell, 41:521, 1985.
Bosze, Thiesen and Charnay, “A transcriptional enhancer with specificity for erythroid cells is located in the long terminal repeat of the friend murine leukemia virus,” EMBO J., 5:1615, 1986.
Bourlais, Acar, Zia, Sado, Needham, Leverge, “Ophthalmic drug delivery systems—recent advances,” Prog. Retin Eye Res., 17(1):33-58, 1998.
Braddock, Chambers, Wilson, Esnouf, Adams, Kingsman and Kingsman, “HIV-I tat activates presynthesized RNA in the nucleus,” Cell, 58:269, 1989.
Bray, “The Zucker-fatty rat: a review,” Fed. Proc., 36:148-53, 1977.
Bulla and Siddiqui, “The Hepatitis B virus enhancer modulates transcription of the Hepatitis B virus surface-antigen gene from an internal location,” J. Virol., 62:1437, 1986.
Buller, Janik, Sebring, Rose, “Herpes simplex virus types 1 and 2 completely help adenovirus-associated virus replication, J. Virol., 40(1):241-247, 1981.
Butler, Kesterson, Khong, Cullen, Pelleymounter, Dekoning, Baetscher and Cone, “A unique metabolic syndrome causes obesity in the melanocortin-3receptor-deficient mouse,” Endocrinology, 141:3518-21, 2000.
Caldovic and Hackett Jr., “Development of position-independent expression vectors and their transfer into transgenic fish,” Mol. Mar. Biol. Biotechnol., 4(1):51-61, 1995.
Campbell and Villarreal, “Functional analysis of the individual enhancer core sequences of polyoma virus: cell-specific uncoupling of DNA replication from transcription,” Mol. Cell. Biol., 8:1993, 1988.
Carnpere and Tilghman, “Postnatal repression of the α-fetoprotein gene is enhancer independent,” Genes and Dev., 3:537, 1989.
Campo, Spandidos, Lang and Wilkie, “Transcriptional control signals in the genome of bovine papilloma virus Type 1,” Nature, 303:77, 1983.
Cannon and Nedergaard, “Brown adipose tissue: function and physiological significance,” Physiol. Rev., 84:277-359, 2004.
Capecchi, “High efficiency transformation by direct microinjection of DNA into cultured mammalian cells,” Cell, 22(2):479-488, 1980.
Carter et al., In: The Parvoviruses, K. I. Berns (Ed.), Plenum, N.Y., pp. 153-207, 1983.
Carver, Dalrymple, Wright, Cottom, Reeves, Gibson, Keenan, Barrass, Scott, Colman, et al., “Transgenic livestock as bioreactors: stable expression of human α-1-antitrypsin by a flock of sheep,” Biotechnology NY, 11(1 1):1263-1270, 1993.
Cech et al., “In vitro splicing of the ribosomal RNA precursor of Tetrahymena: involvement of a guanosine nucleotide in the excision of the intervening sequence,” Cell, 27(3 Pt 2):487-496, 1981.
Cech, Biochem. Int., 18:7-14, 1989.
Cech, Annu. Rev. Biochem., 59:543-69, 1990.
Celander and Haseltine, “Glucocorticoid regulation of murine leukemia virus transcription elements is specified by determinants within the viral enhancer region,” J. Virology, 61:269, 1987.
Celander, Hsu and Haseltine, “Regulatory elements within the murine leukemia virus enhancer regions mediate glucocorticoid responsiveness,” J. Virology, 62:1314, 1988.
Cettour-Rose and Rohner-Jeanrenaud, “The leptin-like effects of 3-d peripheral administration of a melanocortin agonist are more marked in genetically obese Zucker (fa/fa) than in lean rats,” Endocrinology, 143:2277-83, 2002.
Chandler, Maler and Yamamoto, “DNA sequences bound specifically by glucocorticoid receptor in vitro render a heterlogous promoter hormone responsive in vivo,” Cell, 33:489, 1983.
Chandran, Roy, Mishra, “Recent trends in drug delivery systems: liposomal drug delivery system—preparation and characterization,” Indian J. Exp. Biol., 35(8):801-809, 1997.
Chang, Erwin and Lee, “Glucose-regulated protein (GRP94 and GRP78) genes share common regulatory domains and are coordinately regulated by common trans-acting factors,” Mol. Cell. Biol., 9:2153, 1989.
Chao, Liu, Rabinowitz, Li, Samulski and Walsh, “Several log increase in therapeutic transgene delivery by distinct adeno-associated viral serotype vectors,” Mol. Ther., 2:619-23, 2000.
Chatterjee, Lee, Rentoumis and Jameson, “Negative regulation of the thyroid-stimulating hormone α gene by thyroid hormone: Receptor interaction adjacent to the TATA box,” Proc. Natl. Acad. Sci. U.S.A., 86:9114, 1989.
Chen and Okayama, “High-efficiency transformation of mammalian cells by plasmid DNA,” Mol. Cell. Biol., 7:2745-2752, 1987.
Chen et al., “Multitarget-ribozyme directed to cleave at up to nine highly conserved HIV-1 env RNA regions inhibits HIV-1 replication—potential effectiveness against most presently sequenced HIV-1 isolates,” Nuc. Acids Res., 20:4581-4589, 1992.
Cheung, Hoggan, Hauswirth, Berns, “Integration of the adeno-associated virus genome into cellular DNA in latently infected human Detroit 6 cells,” J. Virol., 33(2):739-748, 1980.
Cheung, Clifton and Steiner, “Proopiomelanocortin neurons are direct targets for leptin in the hypothalamus,” Endocrinology, 138:4489-92, 1997.
Choi, Chen, Kriegler and Roninson, “An altered pattern of cross-resistance in multi-drug-resistant human cells results from spontaneous mutations in the MDR-1 (P-glycoprotein) gene,” Cell, 53:519, 1988.
Chowrira and Burke, “Extensive phosphorothioate substitution yields highly active and nuclease-resistant hairpin ribozymes,” Nuc. Acids Res., 20:2835-2840, 1992.
Clark, Voulgaropoulou and Johnson, “A stable cell line carrying adenovirus-inducible rep and cap genes allows for infectivity titration of adeno-associated virus vectors,” Gene Therapy 3:1124-32, 1996.
Clark, Sferra and Johnson, “Recombinant adeno-associated viral vectors mediate long-term transgene expression in muscle,” Hum. Gene Ther., 8:659-69, 1997.
Cohen, Walter and Levinson, “A repetitive sequence element 3′ of the human c-Ha-ras1 gene has enhancer activity,” J. Cell. Physiol., 5:75, 1987.
Collins and Olive, “Reaction conditions and kinetics of self-cleavage of a ribozyme derived from Neurospora VS RNA,” Biochem., 32(11):2795-2799, 1993.
Cone, “The Central Melanocortin System and Energy Homeostasis,” Trends Endocrinol. Metab., 10:211-216,1999.
Conrad, Allen, Afione, Reynolds, Beck, Fee-Maki, Barrazza-Ortiz, Adams, Askin, Carter, Guggino and Flotte, “Safety of single-dose administration of an adeno-associated virus (AAV)-CFTR vector in the primate lung,” Gene Ther., 3:658-68, 1996.
Costa, Lai, Grayson and Damell, “The cell-specific enhancer of the mouse transthyretin (prealbumin) gene binds a common factor at one site and a liver-specific factor(s) at two other sites,” Mol. Cell. Biol., 8:81, 1988.
Coune, “Liposomes as drug delivery system in the treatment of infectious diseases: potential applications and clinical experience,” Infection, 16(3):141-147, 1988.
Couvreur et al., “Nanocapsules, a new lysosomotropic carrier,” FEBS Lett., 84:323-326, 1977.
Couvreur et al., “Tissue distribution of antitumor drugs associated with polyalkylcyanoacrylate nanoparticles,” J. Pharm. Sci., 69(2):199-202, 1980.
Couvreur, “Polyalkyleyanoacrylates as colloidal drug carriers,” Crit. Rev. Ther. Drug Carrier Syst., 5:1-20, 1988.
Cozzi, Tucker, Langford, Pino-Chavez, Wright, O'Connell, Young, Lancaster, McLanghlin, Hunt, Bordin, White, “Characterization of pigs transgenic for human decay-accelerating factor,” Transplantation, 64(10):1383-1392, 1997.
Cripe, Haugen, Turk, Tabatabai, Schmid, Durst, Gissmann, Roman and Turek, “Transcriptional regulation of the human papilloma virus-16 E6-E7 promoter by a keratinocyte-dependent enhancer, and by viral E2 trans-activator and repressor gene products: Implications for cervical carcinogenesis,” EMBO J., 6:3745, 1987.
Cukor, Blacklow, Hoggan and Berns, in THE PARVOVIRUSES, Berns (ed.), Plenum Press, New York, pp. 33-66, 1983.
Cukor et al., In: The Paroviruses, K. I. Berns (Ed.), Plenum, New York, pp. 33-66, 1984.
Culotta and Hamer, “Fine mapping of a mouse metallothionein gene metal-response element,” Mol. Cell. Biol., 9:1376, 1989.
Curiel, Agarwal, Wagner, Cotten, “Adenovirus enhancement of transferrin-polylysine-mediated gene delivery,” Proc. Natl. Acad. Sci. USA, 88(19):8850-8854, 1991.
Daly, Ohlemiller, Roberts, Vogler and Sands, “Prevention of systemic clinical disease in MPS VII mice following AAV-mediated neonatal gene transfer,” Gene Ther., 8:1291-98, 2001.
Dandolo, Blangy and Kamen, “Regulation of polyma virus transcription in murine embryonal carcinoma cells,” J. Virology, 47:55, 1983.
de Villiers, Schaffner, Tyndall, Lupton and Kamen, “Polyoma virus DNA replication requires an enhancer,” Nature, 312:242, 1984.
Deschamps, Meijlink and Verma, “Identification of a transcriptional enhancer element upstream from the proto-oncogene Fos,” Science, 230:1174, 1985.
Dong, Fan and Frizzell, “Quantitative analysis of the packaging capacity of recombinant adeno-associated virus,” Hum. Gene Ther., 7:2101-12, 1996.
Douglas, Davis, Illum, “Nanoparticles in drug delivery,” Crit. Rev. Ther. Drug Carrier Syst., 3(3):233-261, 1987.
Dropulic, Lin, Martin, Jeang, “Functional characterization of a U5 ribozyme: intracellular suppression of human immunodeficiency virus type 1 expression,” J. Virol., 66(3):1432-41, 1992.
Duan, Yue, Yan and Engelhardt, “A new dual-vector approach to enhance recombinant adeno-associated virus-mediated gene expression through intermolecular cis activation,” Nat. Med., 6:595-98, 2000.
Ebert, Selgrath, DiTullio, Denman, Smith, Memon, Schindler, Monastersky, Vitale, Gordon, “Transgenic production of a variant of human tissue-type plasminogen activator in goat milk: generation of transgenic goats and analysis of expression,” Biotechnology NY, 9(9):835-838, 1991.
Edbrooke, Burt, Cheshire and Woo, “Identification of cis-acting sequences responsible for phorbol ester induction of human serum amyloid a gene expression via a nuclear-factor-KB-like transcription factor,” Mol. Cell. Biol., 9:1908, 1989.
Edlund, Walker, Barr and Rutter, “Cell-specific expression of the rat insulin gene: Evidence for role of two distinct 5′ flanking elements,” Science, 230:912, 1985.
Elias, Kelly, Lee, Ahima, Drucker, Saper and Elmquist, “Chemical characterization of leptin-activated neurons in the rat brain,” J. Comp. Neurol., 423:261-81, 2000.
Elroy-Stein and Moss, “Cytoplasmic expression system based on constitutive synthesis of bacteriophage T7 RNA polymerase in mammalian cells,” Proc. Natl. Acad. Sci. USA, 87:6743-7, 1990.
Fan, Boston, Kesterson, Hruby and Cone, “Role of melanocortinergic neurons in feeding and the agouti obesity syndrome,” Nature, 385:165-68, 1997.
Faller and Baltimore, “Liposome encapsulation of retrovirus allows efficient super infection of resistant cell lines,” J. Virol., 49(1):269-272, 1984.
Fallon, “Transgenic insect cells: mosquito cell mutants and the dihydrofolate reductase gene,” Cytotechnology, 20(1-3):23-31, 1996.
Fechheimer et al., “Transfection of mammalian cells with plasmid DNA by scrape loading and sonication loading,” Proc. Natl. Acad. Sci. USA, 84:8463-8467, 1987.
Feng and Holland, “HIV-1 tat trans-activation requires the loop sequence within tar,” Nature, 334:6178, 1988.
Firak and Subramanian, “Minimal transcription enhancer of simian virus 40 is a 74-base-pair sequence that has interacting domains,” Mol. Cell. BioL, 6:3667, 1986.
Fisher, Jooss, Alston, Yang, Haecker, High, Pathak, Raper and Wilson, “Recombinant adeno-associated virus for muscle directed gene therapy,” Nat. Med., 3:306-12, 1997.
Flannery, Zolotukhin, Vaquero, LaVail, Muzyczka and Hauswirth, “Efficient photoreceptor-targeted gene expression in vivo by recombinant adeno-associated virus,” Proc. Natl. Acad. Sci. USA, 94:6916-21, 1997.
Flotte and Carter, “Adeno-associated virus vectors for gene therapy,” Gene Ther., 2:357-62, 1995.
Flotte and Carter, “Adeno-associated virus vectors for gene therapy of cystic fibrosis,” Methods Enzymol., 292:717-32, 1998.
Flotte and Ferkol, “Genetic therapy. Past, present, and future,” Pediatr. Clin. North Am., 44:153-78, 1997.
Flotte, Solow, Owens, Afione, Zeitlin and Carter, “Gene expression from adeno-associated virus vectors in airway epithelial cells,” Am. J. Respir. Cell Mol. Biol., 7:349-56, 1992.
Flotte, Afione, Conrad, McGrath, Solow, Oka, Zeitlin, Guggino and Carter, “Stable in vivo expression of the cystic fibrosis transmembrane conductance regulator with an adeno-associated virus vector,” Proc. Natl. Acad. Sci. USA, 90:10613-17, 1993.
Flotte, Afione and Zeitlin, “Adeno-associated virus vector gene expression occurs in nondividing cells in the absence of vector DNA integration,” Am. J. Respir. Cell Mol. Biol., 11:517-21, 1994.
Flotte, Barraza-Ortiz, Solow, Afione, Carter and Guggino, “An improved system for packaging recombinant adeno-associated virus vectors capable of in vivo transduction,” Gene Ther., 2:29-37, 1995.
Flotte, Carter, Conrad, Guggino, Reynolds, Rosenstein, Taylor, Walden and Wetzel, “A phase I study of an adeno-associated virus-CFTR gene vector in adult CF patients with mild lung disease,” Hum. Gene Ther., 7:1145-59, 1996.
Flotte, Beck, Chesnut, Potter, Poirier and Zolotukhin, “A fluorescence video-endoscopy technique for detection of gene transfer and expression,” Gene Ther., 5:166-73, 1998.
Foecking and Hofstetter, “Powerful and versatile enhancer-promoter unit for mammalian expression vectors,” Gene, 45(1):101-105, 1986.
Forster and Symons, “Self-cleavage of plus and minus RNAs of a virusoid and a structural model for the active sites,” Cell, 49:211-220, 1987.
Fraley et al., “Entrapment of a bacterial plasmid in phospholipid vesicles: Potential for gene transfer,” Proc. Natl. Acad. Sci. USA, 76:3348-3352, 1979.
Franz, Mueller, Haartong, Frey, Katus, “Transgenic animal models: new avenues in cardiovascular physiology,” J. Mol. Med., 75(2):115-119, 1997.
Frederich, Hamann, Anderson, Lollmann, Lowell and Flier, “Leptin levels reflect body lipid content in mice: evidence for diet-induced resistance to leptin action,” Nat. Med., 1:1311-14, 1995.
Fresta and Puglisi, “Application of liposomes as potential cutaneous drug delivery systems. In vitro and in vivo investigation with radioactively labelled vesicles,” J. Drug Target, 4(2):95-101, 1996.
Friedman and Halaas, “Leptin and the regulation of body weight in mammals,” Nature, 395:763-70, 1998.
Frohman, Downs, Kashio, Brinster, “Tissue distribution and molecular heterogeneity of human growth hormone-releasing factor in the transgenic mouse,” Endocrinology, 127(5):2149-2156, 1990.
Fromm, Taylor, Walbot, “Expression of genes transferred into monocot and dicot plant cells by electroporation,” Proc. Natl. Acad. Sci. USA, 82(17):5824-5828, 1985.
Fujita, Shibuya, Hotta, Yamanishi and Taniguchi, “Interferon-β gene regulation: tandemly repeated sequences of a synthetic 6-bp oligomer function as a virus-inducible enhancer,” Cell, 49:357, 1987.
Gabizon and Papahadjopoulos, “Liposomes formulations with prolonged circulation time in blood and enhanced uptake by tumors,” Proc. Natl. Acad. Sci. USA, 85:6949-6953, 1988.
Gabriely, Ma, Yang, Atzmon, Rajala, Berg, Scherer, Rossetti and Barzilai, “Removal of visceral fat prevents insulin resistance and glucose intolerance of aging: an adipokine-mediated process?,” Diabetes, 51:2951-58, 2002.
Gao and Huang, “Cytoplasmic expression of a reporter gene by co-delivery of T7 RNA polymerase and T7 promoter sequence with cationic liposomes,” Nuc. Acids Res., 21:2867-2872, 1993.
Gao, Qu, Faust, Engdahl, Xiao, Hughes, Zoltick and Wilson, “High-titer adeno-associated viral vectors from a Rep/Cap cell line and hybrid shuttle virus,” Hum. Gene Ther., 9:2353-62, 1998.
Gao, Alvira, Wang, Calcedo, Johnston and Wilson, “Novel adeno-associated viruses from rhesus monkeys as vectors for human gene therapy,” Proc. Natl Acad. Sci. USA, 99:11854-59, 2002.
Gerlach et al., “Construction of a plant disease resistance gene from the satellite RNA of tobacco rinspot virus,” Nature (London), 328:802-805, 1987.
Gilles, Morris, Oi and Tonegawa, “A tissue-specific transcription enhancer element is located in the major intron of a rearranged immunoglobulin heavy-chain gene,” Cell, 33:717, 1983.
Giraud, Winocour and Bems, “Site-specific integration by adeno-associated virus is directed by a cellular DNA sequence,” Proc. Natl. Acad. Sci. USA, 91:10039-43, 1994.
Giraud, Winocour and Bems, “Recombinant junctions formed by site-specific integration of adeno-associated virus into an episome,” J. Virol., 69:6917-24, 1995.
Gloss, Bernard, Seedorf and Klock, “The upstream regulatory region of the human papilloma virus-16 contains an E2 protein-independent enhancer which is specific for cervical carcinoma cells and regulated by glucocorticoid hormones,” EMBO J., 6:3735, 1987.
Godbout, Ingram and Tilghman, “Fine-structure mapping of the three mouse a-fetoprotein gene enhancers,” Mol. Cell. Biol., 8:1169, 1988.
Good, Porter, Mahon, Parlow, Westphal and Kirsch, “Hypogonadism and obesity in mice with a targeted deletion of the Nh1h2 gene,” Nat. Genet., 15:397-401, 1997.
Goodboum and Maniatis, “Overlapping positive and negative regulatory domains of the human β-interferon gene,” Proc. Natl. Acad. Sci. USA, 85:1447, 1988.
Goodboum, Burstein and Maniatis, “The human β-interferon gene enhancer is under negative control,” Cell, 45:601, 1986.
Gopal, “Gene transfer method for transient gene expression, stable transfection, and cotransfection of suspension cell cultures,” Mol. Cell Biol., 5:1188-1190, 1985.
Graham and van der Eb, “Transformation of rat cells by DNA of human adenovirus 5,” Virol., 54(2):536-539, 1973.
Greene, Bohnlein and Ballard, “HIV-1, and normal T-cell growth: Transcriptional strategies and surprises,” Immunol. Today, 10:272, 1989.
Grosschedl and Baltimore, “Cell-type specificity of immunoglobulin gene expression is regulated by at least three DNA sequence elements,” Cell, 41:885, 1985.
Guan, Yu and Van der Ploeg, “Evidence of altered hypothalamic pro-opiomelanocortin/neuropeptide Y mRNA expression in tubby mice,” Brain Res. Mol. Brain Res., 59:273-79, 1998.
Guerrier-Takada, Gardiner, Marsh, pace, Altman, “The RNA moiety of ribonuclease P is the catalytic subunit of the enzyme,” Cell, 35:849, 1983.
Gupta, Cases, She, Ma, Yang, Hu, Wu, Rossetti and Barzilai, “Ability of insulin to modulate hepatic glucose production in aging rats is impaired by fat accumulation,” Am. J. Physiol. Endocrinol. Metab., 278:E985-91, 2000.
Halbert, Standaert, Wilson and Miller, “Successful readministration of adeno-associated virus vectors to the mouse lung requires transient immunosuppression during the initial exposure,” J. Virol., 72:9795-805, 1998.
Hampel and Tritz, “RNA catalytic properties of the minimum (−)s TRSV sequence,” Biochem., 28:4929, 1989.
Hampel, Tritz, Hicks, Cruz, “‘Hairpin’ catalytic RNA model: evidence for helices and sequence requirement for substrate RNA,” Nucl. Acids Res., 18:299, 1990.
Handa, Shiroki, Shimojo, “Establishment and characterization of KB cell lines latently infected with adeno-associated virus type 1,” Virol., 82(1):84-92, 1977.
Harland and Weintraub, “Translation of mRNA injected into Xenopus oocytes is specifically inhibited by antisense RNA,” J. Cell Biol., 101(3):1094-1099, 1985.
Harrold, Widdowson and Williams, “Altered energy balance causes selective changes in melanocortin-4(MC4-R), but not melanocortin-3 (MC3-R), receptors in specific hypothalamic regions: further evidence that activation of MC4-R is a physiological inhibitor of feeding,” Diabetes, 48:267-71, 1999.
Harrold, Widdowson and Williams, “beta-MSH: a functional ligand that regulated energy homeostasis via hypothalamic MC4-R?.” Peptides, 24:397-405, 2003.
Haskell and Bowen, “Efficient production of transgenic cattle by retroviral infection of early embryos,” Mol. Reprod. Dev., 40(3):386-390, 1995.
Haslinger and Karin, “Upstream promoter element of the human metallothionein-II gene can act like an enhancer element,” Proc. Natl. Acad. Sci. USA, 82:8572, 1985.
Hauber and Cullen, “Mutational analysis of the trans-activation-responsive region of the human immunodeficiency virus Type I long terminal repeat,” J. Virol., 62:673, 1988.
Heath and Martin, “The development and application of protein-liposome conjugation techniques,” Chem. Phys. Lipids, 40:347-358, 1986.
Heath et al., “Liposome-mediated delivery of pteridine antifolates to cells: in vitro potency of methotrexate and its a and gamma substituents,” Biochim. Biophys. Acta, 862:72-80, 1986.
Hen, Borrelli, Fromental, Sassone-Corsi and Chambon, “A mutated polyoma virus enhancer which is active in undifferentiated embryonal carcinoma cells is not repressed by adenovirus-2 E1A products,” Nature, 321:249, 1986.
Henry-Michelland et al., “Attachment of antibiotics to nanoparticles; Preparation, drug-release and antimicrobial activity in vitro,” Int. J. Pharm., 35:121-127, 1987.
Hensel, Meichle, Pfizenmaier and Kronke, “PMA-responsive 5′ flanking sequences of the human TNF gene,” Lymphokine Res., 8:347, 1989.
Hermonat and Muzyczka, “Use of adeno-associated virus as a mammalian DNA cloning vector: transduction of neomycin resistance into mammalian tissue culture cells,” Proc. Natl. Acad. Sci. USA, 81:6466-70, 1984.
Hermonat, Labow, Wright, Bems, Muzyczka, “Genetics of adeno-associated virus: isolation and preliminary characterization of adeno-associated virus type 2 mutants,” J. Virol., 51(2):329-339, 1984.
Hernandez, Wang, Kearns, Loiler, Poirier and Flotte, “Latent adeno-associated virus infection elicits humoral but not cell-mediated immune responses in a nonhuman primate model,” J. Virol., 73:8549-58, 1999.
Herr and Clarke, “The SV40 enhancer is composed of multiple functional elements that can compensate for one another,” Cell, 45:461, 1986.
Hildinger, Auricchio, Gao, Wang, Chirmule and Wilson, “Hybrid vectors based on adeno-associated virus serotypes 2 and 5 for muscle-directed gene transfer,” J. Virol., 75:6199-203, 2001.
Hirochika, Browker and Chow, “Enhancers and trans-acting E2 transcriptional factors of papilloma viruses,” J. Virol., 61:2599, 1987.
Hirsch, Gaugler, Deagostini-Bauzin, Bally-Cuif and Gordis, “Identification of positive and negative regulatory elements governing cell-type-specific expression of the neural-cell-adhesion-molecule gene,” Mol. Cell. Biol., 10:1959, 1990.
Hoggan et al., In: Proceeding of the Fourth Lepetit Colloquium, Cacoyac, Mexico, North Holland, Amsterdam, pp. 243-249, 1972.
Hoggan, Fed. Proc., 24:248, 1965.
Holbrook, Gulino and Ruscetti, “cis-acting transcriptional regulatory sequences in the Gibbon ape leukemia virus (GALV) long terminal repeat,” Virology, 157:211, 1987.
Hoover et al., Eds., In: Remington's Pharmaceutical Sciences, 16^{th} Edition, Mack Publishing Co., Easton, Pa., 1980.
Horlick and Benfield, “The upstream muscle-specific enhancer of the rat muscle creatine kinase gene is composed of multiple elements,” Mol. Cell. Biol., 9:2396, 1989.
Horton, Goldstein and Brown, “SREBPs: activators of the complete program of cholesterol and fatty acid synthesis in the liver,” J. Clin. Invest., 109:1125-31, 2002.
Huang, Ostrowski, Berard and Hagar, “Glucocorticoid regulation of the Ha-MuSV p21 gene conferred by sequences from mouse mammary tumor virus,” Cell, 27:245, 1981.
Huszar, Lynch, Fairchild-Huntress, Dunmore, Fang, Berkemeier, Gu, Kesterson, Boston, Cone, Smith, Campfield, Bum and Lee, “Targeted disruption of the melanocortin-4 receptor results in obesity in mice,” Cell, 88:131-41, 1997.
Hwa, Ghibaudi, Gao and Parker, “Central melanocortin system modulates energy intake and expenditure of obese and lean Zucker rats,” Am. J. Physiol. Regul. Integr. Comp. Physiol., 281:R444-51, 2001.
Hwang, Lim and Chae, “Characterization of the s-phase-specific transcription regulatory elements in a DNA-replication-independent testis-specific H2B (TH2B) histone gene,” Mol. Cell. Biol., 10:585, 1990.
Iida, Murakami, Ishida, Mizuno, Kuwajima and Shima, “Substitution at codon 269 (glutamine -->proline) of the leptin receptor (OB-R) cDNA is the only mutation found in the Zucker fatty (fa/fa) rat,” Biochem. Biophys. Res. Commun., 224:597-604, 1996.
Im and Muzyczka, “The AAV origin binding protein Rep68 is an ATP-dependent site-specific endonuclease with DNA helicase activity,” Cell, 61:447-57, 1990.
Imagawa, Chiu and Karin, “Transcription factor AP-2 mediates induction by two different signal-transduction pathways: Protein kinase C and cAMP,” Cell, 51:251, 1987.
Imaizumi et al., “Liposome-entrapped superoxide dismutase reduces cerebral infarction in cerebral ischemia in rats,” Stroke, 21(9):1312-1317, 1990a.
Imaizumi et al., “Liposome-entrapped superoxide dismutase ameliorates infarct volume in focal cerebral ischemia,” Acta. Neurochirurgica Suppl., 51:236-238, 1990b.
Imbra and Karin, “Phorbol ester induces the transcriptional stimulatory activity of the SV40 enhancer,” Nature, 323:555, 1986.
Imler, Lemaire, Wasvlyk and Waslyk, “Negative regulation contributes to tissue specificity of the immunoglobulin heavy-chain enhancer,” Mol. Cell. Biol., 7:2558, 1987.
Imperiale and Nevins, “Adenovirus 5 E2 transcription unit: An E1A-inducible promoter with an essential element that functions independently of position or orientation,” Mol. Cell. Biol., 4:875, 1984.
Jaeger, Turner and Zuker, “Improved predictions of secondary structures for RNA,” Proc. Natl. Acad. Sci. USA, 86:7706-10, 1989.
Jakobovits, Smith, Jakobovits and Capon, “A discrete element 3′ of human immunodeficiency virus 1 (HIV-1) and HIV-2 mRNA initiation sites mediates transcriptional activation by an HIV trans-activator,” Mol. Cell. Biol., 8:2555, 1988.
Jameel and Siddiqui, “The human Hepatitis B virus enhancer requires transacting cellular factor(s) for activity,” Mol. Cell. Biol., 6:710, 1986.
Jaynes, Johnson, Buskin, Gartside and Hauschka, “The muscle creatine kinase gene is regulated by multiple upstream elements, including a muscle-specific enhancer,” Mol. Cell. Biol., 8:62, 1988.
Johnson, Wold and Hauschka, “Muscle creatine kinase sequence elements regulating skeletal and cardiac muscle expression in transgenic mice,” Mol. Cell. Biol., 9:3393, 1989.
Jooss, Yang, Fisher and Wilson, “Transduction of dendritic cells by DNA viral vectors directs the immune response to transgene products in muscle fibers,” J. Virol., 727:4212-23, 1998.
Joyce, “RNA evolution and the origins of life,” Nature, 338:217-244, 1989.
Kadesch and Berg, “Effects of the position of the simian virus 40 enhancer on expression of multiple transcription units in a single plasmid,” Mol. Cell. Biol., 6:2593, 1986.
Kang, et al., “Up-regulation of luciferase gene expression with antisense oligonucleotides: implications and applications in functional assay development,” Biochemistry, 37(18):6235-9, 1998.
Kaplitt, Leone, Samulski, Xiao, Pfaff, O'Malley and During, “Long-term gene expression and phenotypic correction using adeno-associated virus vectors in the mammalian brain,” Nat. Genet., 8:148-54, 1994.
Karin, Haslinger, Heguy, Dietlin and Cooke, “Metal-responsive elements act as positive modulators of human metallothionein-IIa enhancer activity,” Mol. Cell. Biol., 7:606, 1987.
Kashani-Sabet et al., “Reversal of the malignant phenotype by an anti-ras ribozyme,” AntisenseRes. Dev., 2:3-15, 1992.
Katinka, Vasseur, Montreau, Yaniv and Blangy, “Polyoma DNA sequences involved in the control of viral gene expression in murine embryonal carcinoma cells,” Nature, 290:720, 1981.
Katinka, Yaniv, Vasseur and Blangy, “Expression of polyoma early functions in mouse embryonal carcinoma cells depends on sequence rearrangements in the beginning of the late region,” Cell, 20:393, 1980.
Kawamoto, Makino, Niw, Sugiyama, Kimura, Anemura, Nakata and Kakunaga, “Identification of the human β-actin enhancer and its binding factor,” Mol. Cell. Biol., 8:267, 1988.
Kay, Manno, Ragni, et al., “Evidence for gene transfer and expression of factor IX in haemophilia B patients treated with an AAV vector,” Nat. Genet., 24:257-261, 2000.
Kearns, Afione, Fulmer, Pang, Erikson, Egan, Landrum, Flotte and Cutting, “Recombinant adeno-associated virus (AAV-CFTR) vectors do not integrate in a site-specific fashion in an immortalized epithelial cell line,” Gene Ther., 3:748-55, 1996.
Kessler, Podsakoff, Chen, McQuiston, Colosi, Matelis, Kurtzman and Byrne, “Gene delivery to skeletal muscle results in sustained expression and systemic delivery of a therapeutic protein,” Proc. Natl. Acad. Sci. USA, 93:14082-87, 1996.
Kiledjian, Su and Kadesch, “Identification and characterization of two functional domains within the murine heavy-chain enhancer,” Mol. Cell. Biol., 8:145, 1988.
Kim and Cech, “Three-dimensional model of the active site of the self-splicing rRNA precursor of Tetrahymena,” Proc. NatL. Acad. Sci. USA 84:8788-8792, 1987.
Kim, O'Hare, Grace, Welch, Billington and Levine, “ARC POMC MRNA and PVN alpha-MSH are lower in obese relative to lean zucker rats,” Brain Res., 862:11-16, 2000.
Klamut, Gangopadyhay, Worton and Ray, “Molecular and functional analysis of the muscle-specific promoter region of the Duchenne muscular dystrophy gene,” Mol. Cell. Biol., 10:193, 1990.
Klein, Wolf, Wu, Sanford, “High-velocity microprojectiles for delivering nucleic acids into living cells. 1987,” Biotechnology, 24:384-386, 1992.
Klein, Meyer, Peel, Zolotukhin, Meyers, Muzyczka and King, “Neuron-specific transduction in the rat septohippocampal or nigrostriatal pathway by recombinant adeno-associated virus vectors,” Exper. Neurol. 150:183-94, 1998.
Koch, Benoist and Mathis, “Anatomy of a new B-cell-specific enhancer,” Mol. Cell. Biol., 9:303, 1989.
Koeberl, Alexander, Halbert, et al., “Persistent expression of human clotting factor IX from mouse liver after intravenous injection of adeno-associated virus vectors,” Proc. Nat'l Acad. Sci. USA, 94:1426-1431, 1997.
Koizumi, Kamiya and Ohtsuka, Gene, 117:179-84, 1992.
Kotin, “Prospects for the use of adeno-associated virus as a vector for human gene therapy,” Hum. Gene Ther., 5:793-801, 1994.
Kotin, Siniscalco, Samulski, Zhu, Hunter, Laughlin, McLaughlin, Muzyczka, Rocchi and Berns, “Site-specific integration by adeno-associated virus,” Proc. Natl. Acad. Sci. USA, 87:2211-15, 1990.
Kotin, Menninger, Ward and Berns, “Mapping and direct visualization of a region-specific viral DNA integration site on chromosome 19q13-qter,” Genomics, 10:831-34, 1991.
Kotin, Linden and Berns, “Characterization of a preferred site on human chromosome 19q for integration of adeno-associated virus DNA by non-homologous recombination,” EMBO Journal, 11:5071-78, 1992.
Kriegler and Botchan, “A retrovirus LTR contains a new type of eukaryotic regulatory element,” In: Eukaryotic Viral Vectors, Gluzman, Ed., Cold Spring Harbor, Cold Spring Harbor Laboratory, New York, 1982.
Kriegler and Botchan, “Enhanced transformation by a simian virus 40 recombinant virus containing a Harvey murine sarcoma virus long terminal repeat,” Mol. Cell. Biol. 3:325, 1983.
Kriegler, Perez and Botchan, “Promoter substitution and enhancer augmentation increases the penetrance of the SV40 a gene to levels comparable to that of the Harvey murine sarcoma virus Ras gene in morphologic transformation,” In: Gene Expression, Hamer and Rosenberg, Eds., New York, Alan R. Liss, 1983.
Kriegler, Perez, Defay, Albert and Liu, “A novel form of TNF/cachectin is a cell-surface cytotoxix transmembrane protein: Ramifications for the complex physiology of TNF,” Cell, 53:45, 1988.
Kriegler, Perez, Hardy and Botchan, “Transformation mediated by the SV40 T antigens: separation of the overlapping SV40 early genes with a retroviral vector,” Cell, 38:483, 1984a.
Kriegler, Perez, Hardy and Botchan, “Viral integration and early gene expression both affect the efficiency of SV40 transformation of murine cells: Biochemical and biological characterization of an SV40 retrovirus,” In: Cancer Cells 2/Oncogenes and Viral Genes, Van de Woude, Levine, Topp and Watson, Eds., Cold Spring Harbor, Cold Spring Harbor Laboratory, 1984b.
Krude, Biebermann, Luck, Horn, Brabant and Gruters, “Severe early-onset obesity, adrenal insufficiency and red hair pigmentation caused by POMC mutations in humans,” Nat. Genet., 19:155-57, 1998.
Kuby, In: Immunology, 2nd Edition. W.H. Freeman & Company, New York, 1994.
Kuhl, De La Fuenta, Chaturvedi, Parinool, Ryals, Meyer and Weissman, “Reversible silencing of enhancers by sequences derived from the human IFN-α promoter,” Cell, 50:1057, 1987.
Kunz, Zimmerman, Heisig and Heinrich, “Identification of the promoter sequences involved in the Interleukin-6-dependent expression of the rat a-2-macroglobulin gene,” Nucl. Acids Res., 17:1121, 1989.
Kwoh, Davis, Whitfield, Chappelle, DiMichele, Gingeras, “Transcription-based amplification system and detection of amplified human immunodeficiency virus type 1 with a bead-based sandwich hybridization format,” Proc. NatL. Acad. Sci. USA, 86(4):1173-1177, 1989.
Kyte and Doolittle, “A simple method for displaying the hydropathic character of a protein,” J. Mol. Biol., 157(1):105-132, 1982.
L'Huillier, David, Bellamy, “Cytoplasmic delivery of ribozymes leads to efficient reduction in alpha-lactalbumin MRNA levels in C1271 mouse cells,” EMBO J., 11(12):4411-4418, 1992.
Larkin, Reynolds, Supiano, Kahn and Halter, “Effect of aging and obesity on insulin responsiveness and glut-4 glucose transporter content in skeletal muscle of Fischer 344×Brown Norway rats,” J. Gerontol. A Biol. Sci. Med. Sci., 56:B486-92, 2001.
Larsen, Hamey and Moore, “Repression mediates cell-type-specific expression of the rat growth hormone gene,” Proc. Natl. Acad. Sci. U.S.A., 83:8283, 1986.
Lasic, “Novel applications of liposomes,” Trends Biotechnol., 16(7):307-321, 1998.
Laspia, Rice and Mathews, “HIV-1 tat protein increases transcriptional initiation and stabilizes elongation,” Cell, 59:283, 1989.
Latimer, Berger and Baumann, “Highly conserved upstream regions of the α₁-antitrypsin gene in two mouse species govern liver-specific expression by different mechanisms,” Mol. Cell. Biol., 10:760, 1990.
Laughlin, Tratschin, Coon, Carter, “Cloning of infectious adeno-associated virus genomes in bacterial plasmids,” Gene, 23(1):65-73, 1983.
Lee et al., “Functional analysis of the steroid hormone control region of mouse mammary tumor virus,” Nucleic Acids Res., 12(10):4191-4206, 1984.
Lee, Mulligan, Berg and Ringold, “Glucocorticoids regulate expression of dihydrofolate reductase cDNA in mouse mammary tumor virus chimaeric plasmids,” Nature, 294:228, 1981.
Levin, Finnegan, Marquet and Sullivan, “Defective brown adipose oxygen consumption in obese Zucker rats,” Am. J. Physiol., 247:E94-100, 1984.
Levinson, Khoury, VanDeWoude and Gruss, “Activation of SV40 genome by 72-base-pair tandem repeats of Moloney sarcoma virus,” Nature, 295:79, 1982.
Lewin, Drenser, Hauswirth, Nishikawa, Yasumura, Flannery and LaVail, “Ribozyme rescue of photoreceptor cells in a transgenic rat model of autosomal dominant retinitis pigmentosa,” Nat. Med., 4:967-971, 1998.
Li, Matheny, Nicolson, Turner and Scarpace, “Leptin gene expression increases with age independent of increasing adiposity in rats,” Diabetes, 46:2035-39, 1997.
Li, Klein, Matheny, King, Meyer and Scarpace, “Induction of uncoupling protein 1 by central interleukin-6 gene delivery is dependent on sympathetic innervation of brown adipose tissue and underlies one mechanism of body weight reduction in rats,” Neuroscience, 115:879-89, 2002.
Li, Mobbs and Scarpace, “Central pro-opiomelanocortin gene delivery results in hypophagia, reduced visceral adiposity, and improved insulin sensitivity in genetically obese Zucker rats,” Diabetes, 52:1951-57, 2003.
Lieber, Sandig, Sommer, Bahring, Strauss, “Stable high-level gene expression in mammalian cells by T7 phage RNA polymerase,” Methods Enzymol., 217:47-66, 1993.
Lin, Cross, Halden, Dragos, Toledano and Leonard, “Delineation of an enhancer like positive regulatory element in the interleukin-2 receptor α-chain gene,” Mol. Cell. Biol., 10:850, 1990.
Linden, Ward, Giraud, Winocour and Bems, “Site-specific integration by adeno-associated virus,” Proc. Natl. Acad. Sci. USA, 93:11288-94, 1996.
Lisziewicz et al., “Inhibition of human immunodeficiency virus type 1 replication by regulated expression of a polymeric Tat activation response RNA decoy as a strategy for gene therapy in AIDS,” Proc. Natl. Acad. Sci. USA, 90:8000-8004, 1993.
Little and Lee, J. Biol. Chem., 270:9526-34, 1995.
Loeb, Cordier, Harris, Weitzman and Hope, “Enhanced expression of transgenes from adeno-associated virus vectors with the woodchuck hepatitis virus posttranscriptional regulatory element: implications for gene therapy,” Hum. Gene Ther., 10:2295-305, 1999.
Lopez-Berestein et al., “Liposomal amphotericin B for the treatment of systemic fungal infections in patients with cancer: a preliminary study” J. Infect. Dis., 2151:704, 1985a.
Lopez-Berestein et al., “Protective effect of liposomal-amphotericin B against C. albicans infection in mice,” Cancer Drug Delivery, 2:183, 1985b.
Luria, Gross, Horowitz and Givol, “Promoter enhancer elements in the rearranged a-chain gene of the human T-cell receptor,” EMBO J., 6:3307, 1987.
Lusby and Berns, “Mapping of the 5′ termini of two adeno-associated virus 2 RNAs in the left half of the genome,” J. Virol., 41(2):518-526, 1982.
Lusby, Fife and Berns, “Nucleotide sequence of the inverted terminal repetition in adeno-associated virus DNA,” J. Virol., 34:402-09, 1980.
Lusky and Botchan, “Transient replication of bovine papilloma virus Type 1 plasmids: cis and trans requirements,” Proc. Natl. Acad. Sci. U.S.A., 83:3609, 1986.
Lusky, Berg, Weiher and Botchan, “Bovine papilloma virus contains an activator of gene expression at the distal end of the early transcription unit,” Mol. Cell. Biol. 3:1108, 1983.
Majors and Varmus, “A small region of the mouse mammary tumor virus long terminal repeat confers glucocorticoid hormone regulation on a linked heterologous gene,” Proc. Natl. Acad. Sci. US.A., 80:5866, 1983.
Maloy et al., In: Microbial Genetics, 2nd Edition, Jones and Barlett Publishers, Boston, Mass., 1994.
Mandel, Spratt, Snyder and Leff, “Midbrain injection of recombinant adeno-associated virus encoding rat glial cell line-derived neurotrophic factor protects nigral neurons in a progressive 6-hydroxydopamine-induced degeneration model of Parkinson's disease in rats,” Proc. Natl. Acad. Sci. USA, 94:14083-88, 1997.
Maniatis et al., “Molecular Cloning: a Laboratory Manual,” Cold Spring Harbor Laboratory, Cold Spring Harbor, N.Y., 1982.
Margalit, “Liposome-mediated drug targeting in topical and regional therapies,” Crit. Rev. Ther. Drug Carrier Syst., 12(2-3):233-261, 1995.
Marks, Butler, Turner, Brookhart and Cone, “Differential role of melanocortin receptor subtypes in cachexia,” Endocrinology, 144:1513-23, 2003.
McCown, Xiao, Li, Breese and Samulski, “Differential and persistent expression patterns of CNS gene transfer by an adeno-associated virus (AAV) vector,” Brain Res., 713:99-107, 1996.
McMinn, Wilkinson, Havel, Woods and Schwartz, “Effect of intracerebroventricular alpha-MSH on food intake, adiposity, c-Fos induction, and neuropeptide expression,” Am. J. Physiol. Regul. Integr. Comp. Physiol., 279:R695-703, 2000.
McNeall, Sanchez, Gray, Chesterman and Sleigh, “Hyperinducible gene expression from a metallotionein promoter containing additional metal-responsive elements,” Gene, 76:81, 1989.
Miao, Snyder, Schowalter, Patijn, Donahue, Winther and Kay, “The kinetics of rAAV integration in the liver [letter],” Nat. Genet., 19:13-15, 1998.
Michel and Westhof, “Modeling of the three-dimensional architecture of group I catalytic introns based on comparative sequence analysis,” J. Mol. Biol., 216:585-610, 1990.
Miksicek, Heber, Schmid, Danesch, Posseckert, Beato and Schutz, “Glucocorticoid responsiveness of the transcriptional enhancer of Moloney murine sarcoma virus,” Cell, 46:203, 1986.
Mizuno and Mobbs, “Hypothalamic agouti-related protein messenger ribonucleic acid is inhibited by leptin and stimulated by fasting,” Endocrinology, 140:814-17, 1999.
Mizuno, Kleopoulos, Bergen, Roberts, Priest and Mobbs, “Hypothalamic pro-opiomelanocortin mRNA is reduced by fasting and [corrected] in ob/ob and db/db mice, but is stimulated by leptin,” Diabetes, 47:294-97, 1998.
Mobbs, Bray, Atkinson, Bartke, Finch, Maratos-Flier, Crawley and Nelson, “Neuroendocrine and pharmacological manipulations to assess how caloric restriction increases life span,” J. Gerontol. A Biol. Sci. Med. Sci., 56 Spec No 1:34-44, 2001.
Monahan and Samulski, “AAV vectors: is clinical success on the horizon?,” Gene Ther., 7:24-30, 2000.
Montminy, Gonzalez and Yamamoto, “Regulation of cAMP-inducible genes by CREB,” Trends Neurosci., 13:184-88, 1990.
Mordacq and Linzer, “Co-localization of elements required for phorbol ester stimulation and glucocorticoid repression of proliferin gene expression,” Genes and Dev., 3:760, 1989.
Moreau, Hen, Wasylyk, Everett, Gaub and Chambon, “The SV40 base-repair repeat has a striking effect on gene expression both in SV40 and other chimeric recombinants,” Nucl. Acids Res., 9:6047, 1981.
Mori and Fukatsu, “Anticonvulsant effect of DN-1417 a derivative of thyrotropin-releasing hormone and liposome-entrapped DN-1417 on amygdaloid-kindled rats,” Epilepsia, 33(6):994-1000, 1992.
Muesing, Smith and Capon, “Regulation of mRNA accumulation by a human immunodeficiency virus trans-activator protein,” Cell, 48:691, 1987.
Muller et al., “Efficient transfection and expression of heterologous genes in PC12 cells,” Cell, Biol., 9(3):221-229, 1990.
Muzyczka, “Use of adeno-associated virus as a general transduction vector for mammalian cells,” Curr. Top Microbiol. Immunol., 158:97-129, 1992.
Muzyczka and McLaughlin, “Use of adeno-associated virus as a mammalian transduction vector,” In: Current Communications in Molecular Biology: Viral Vectors, Glazman and Hughes, eds., Cold Spring Harbor Laboratory Press, Cold Spring Harbor, N.Y., pp. 39-44, 1988.
Ng, Gunning, Liu, Leavitt and Kedes, “Regulation of the human β-actin promoter by upstream and intron domains,” Nuc. Acids Res., 17:601, 1989.
Ni, Zhou, McCarty, Zolotukhin and Muzyczka, “In vitro replication of adeno-associated virus DNA,” J. Virol., 68:1128-38, 1994.
Nicolau and Gersonde, “Incorporation of inositol hexaphosphate into intact red blood cells, I. fusion of effector-containing lipid vesicles with erythrocytes,” Naturwissenschaften (Germany), 66(11):563-566, 1979.
Nicolau and Sene, “Liposome-mediated DNA transfer in eukaryotic cells,” Biochem. Biophys. Acta, 721:185-190, 1982.
Obici, Feng, Tan, Liu, Karkanias and Rossetti, “Central melanocortin receptors regulate insulin action,” J. Clin. Invest., 108:1079-85, 2001.
Ohara, Dort, Gilbert, “One-sided polymerase chain reaction: the amplification of cDNA,” Proc. Natl. Acad. Sci. USA, 86(15):5673-5677, 1989.
Ohkawa, Yuyama, Taira, “Activities of HIV-RNA targeted ribozymes transcribed from a ‘shot-gun’ type ribozyme-trimming plasmid,” Nuc. Acids Symp. Ser., 27:15-16, 1992.
Ojwang, Hampel, Looney, Wong-Staal, Rappaport, “Inhibition of human immunodeficiency virus type 1 expression by a hairpin ribozyme,” Proc. Natl. Acad. Sci. USA, 89(22):10802-10806, 1992.
Ondek, Sheppard and Herr, “Discrete elements within the SV40 enhancer region display different cell-specific enhancer activities,” EMBO J., 6:1017, 1987.
Ono, Hirose, Miyazaki, Yamamoto, Matsumoto, “Transgenic medaka fish bearing the mouse tyrosinase gene: expression and transmission of the transgene following electroporation of the orange-colored variant,” Pigment Cell Res., 10(3):168-175, 1997.
Omitz, Hammer, Davison, Brinster and Palmiter, “Promoter and enhancer elements from the rat elastase I gene function independently of each other and of heterologous enhancers,” Mol. Cell. Biol., 7:3466, 1987.
Palmiter, Chen and Brinster, “Differential regulation of metallothionein-thymidine kinase fusion genes in transgenic mice and their offspring,” Cell, 29:701, 1982.
Parks, Melnick, Rongey, Mayor, “Physical assay and growth cycle studies of a defective adeno-satellite virus,” J. Virol., 1(1):171-180, 1967.
Pech, Rao, Robbins and Aaronson, “Functional identification of regulatory elements within the promoter region of platelet-derived growth factor 2,” Mol. Cell. Biol., 9:396, 1989.
Peel, Zolotukhin, Schrimsher, Muzyczka and Reier, “Efficient transduction of green fluorescent protein in spinal cord neurons using adeno-associated virus vectors containing cell type- specific promoters,” Gene Ther., 4:16-24, 1997.
Pereira and Muzyczka, “The cellular transcription factor SP1 and an unknown cellular protein are required to mediate Rep protein activation of the adeno-associated virus p19 promoter,” J. Virol., 71:1747-56, 1997.
Pereira, McCarty and Muzyczka, “The adeno-associated virus (AAV) Rep protein acts as both a repressor and an activator to regulate AAV transcription during a productive infection,” J. Virol., 71:1079-88, 1997.
Perez-Stable and Constantini, “Roles of fetal γ-globin promoter elements and the adult β-globin 3′ enhancer in the stage-specific expression of globin genes,” Mol. Cell. Biol., 10:1116, 1990.
Perreault, Wu, Cousinequ, Ogilvie, Cedergren, “Mixed deoxyribo- and ribo-oligonucleotides with catalytic activity,” Nature, 344(6266):565, 1990.
Perrotta and Been, “Cleavage of oligoribonucleotides by a ribozyme derived from the hepatitis delta virus RNA sequence,” Biochem., 31(1):16, 1992.
Philip, et al., “Efficient and sustained gene expression in primary T lymphocytes and primary and cultured tumor cells mediated by adeno-associated virus plasmid DNA complexed to cationic liposomes,” Mol. Cell Biol., (14)4:2411-8, 1994.
Picard and Schaffner, “A Lymphocyte-specific enhancer in the mouse immunoglobulin kappa gene,” Nature, 307:83, 1984.
Pieken, Olsen, Benseler, Aurup, Eckstein, “Kinetic characterization of ribonuclease-resistant 2′-modified hammerhead ribozymes,” Science, 253(5017):314, 1991.
Pierroz, Ziotopoulou, Ungsunan, Moschos, Flier and Mantzoros, “Effects of acute and chronic administration of the melanocortin agonist MTII in mice with diet-induced obesity,” Diabetes, 51:1337-45, 2002.
Pikul et al., “In vitro killing of melanoma by liposome-delivered intracellular irradiation, Arch. Surg., 122(12):1417-1420, 1987.
Pinkert, Omitz, Brinster and Palmiter, “An albumin enhancer located 10 kb upstream functions along with its promoter to direct efficient, liver-specific expression in transgenic mice,” Genes and Dev., 1:268, 1987.
Pinto-Alphandary, Balland, Couvreur, “A new method to isolate polyalkylcyanoacrylate nanoparticle preparations,” J. Drug Target, 3(2):167-169, 1995.
Pinto-Sietsma and Paul, “Transgenic rats as models for hypertension,” J. Hum. Hypertens., 11(9):577-581, 1997.
Ponta, Kennedy, Skroch, Hynes and Groner, “Hormonal response region in the mouse mammary tumor virus long terminal repeat can be dissociated from the proviral promoter and has enhancer properties,” Proc. Natl. Acad. Sci. U.S.A., 82:1020, 1985.
Porton, Zaller, Lieberson and Eckhardt, “Immunoglobulin heavy-chain enhancer is required to maintain transfected γ2A gene expression in a pre-B-cell line,” Mol. Cell. Biol., 10:1076, 1990.
Potter et al., “Enhancer-dependent expression of human K immunoglobulin genes introduced into mouse pre-B lymphocytes by electroporation,” Proc. Natl. Acad. Sci. USA, 81:7161-7165, 1984.
Pritchard, Turnbull and White, “Pro-opiomelanocortin processing in the hypothalamus: impact on melanocortin signaling and obesity,” J. Endocrinol., 172:411-21, 2002.
Prokop and Bajpai, “Recombinant DNA Technology I,” Conference on Progress in Recombinant DNA Technology Applications, Potosi, Mich., Jun. 3-8, 1990, Ann. N.Y. Acad. Sci., 646:1-383, 1991.
Qing, Mah, Hansen, Zhou, Dwarki and Srivastava, “Human fibroblast growth factor receptor 1 is a co-receptor for infection by adeno-associated virus 2,” Nat. Med., 5:71-77, 1999.
Queen and Baltimore, “Immunoglobulin gene transcription is activated by downstream sequence elements,” Cell, 35:741, 1983.
Quinn, Farina, Gardner, Krutzsch and Levens, “Multiple components are required for sequence recognition of the AP1 site in the Gibbon ape leukemia virus enhancer,” Mol. Cell. Biol., 9:4713, 1989.
Quintanar-Guerrero, Allemann, Doelker, Fessi, “Preparation and characterization of nanocapsules from preformed polymers by a new process based on emulsification-diffusion technique,” Phamr. Res., 15(7):1056-1062, 1998.
Redondo, Hata, Brocklehurst and Krangel, “A T-cell-specific transcriptional enhancer within the human T-cell receptor 6 locus,” Science, 247:1225, 1990.
Reinhold-Hurek and Shub, “Self-splicing introns in tRNA genes of widely divergent bacteria,” Nature, 357:173-176, 1992.
Reisman and Rotter, “Induced expression from the Moloney murine leukemia virus long terminal repeat during differentiation of human myeloid cells is mediated through its transcriptional enhancer,” Mol. Cell. Biol., 9:3571, 1989.
Renneisen et al., “Inhibition of expression of human immunodeficiency virus-1 in vitro by antibody-targeted liposomes containing antisense RNA to the env region,” J. Biol. Chem., 265(27):16337-16342, 1990.
Resendez Jr., Wooden and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.
Ripe, Lorenzen, Brenner and Breindl, “Regulatory elements in the 5′ flanking region and the first intron contribute to transcriptional control of the mouse α-1-type collagen gene,” Mol. Cell. Biol., 9:2224, 1989.
Rippe et al., “DNA-mediated gene transfer into adult rat hepatocytes in primary culture,” Mol. Cell Biol., 10:689-695, 1990.
Rittling, Coutinho, Amarm and Kolbe, “AP-1/jun-binding sites mediate serum inducibility of the human vimentin promoter,” Nuc. Acids Res., 17:1619, 1989.
Rolling, Shen, Tabarias, Constable, Kanagasingam, Barry and Rakoczy, “Evaluation of adeno-associated virus-mediated gene transfer into the rat retina by clinical fluorescence photography,” Hum. Gene Ther., 10:641-48, 1999.
Rose and Koczot, “Adenovirus-associated virus multiplication. VII. Helper requirement for viral deoxyribonucleic acid and ribonucleic acid synthesis,” J. Virol., 10(1): 1-8, 1972.
Rose, Bems, Hoggan, Koczot, “Evidence for a single-stranded adenovirus-associated virus genome: formation of a DNA density hybrid on release of viral DNA,” Proc. Natl. Acad. Sci. USA, 64(3):863-869, 1969.
Rosen, Sodroski and Haseltine, “The location of cis-acting regulatory sequences in the human T-cell lymphotropic virus type III (HTLV-111/LAV) long terminal repeat,” Cell, 41:813, 1988.
Rossi, Elkins, Zaia, Sullivan, “Ribozymes as anti-HIV-1 therapeutic agents: principles, applications, and problems,” AIDS Res. Hum. Retrovir., 8(2):183, 1992.
Sakai, Helms, Carlstedt-Duke, Gustafsson, Rottman and Yamamoto, “Hormone-mediated repression: A negative glucocorticoid-response element from the bovine prolactin gene,” Genes and Dev., 2:1144, 1988.
Sambrook et al., “Molecular Cloning: A Laboratory Manual,” Cold Spring Harbor Laboratory, Cold Spring Harbor, N.Y., 1989.
Samulski, “Adeno-associated virus: integration at a specific chromosomal locus,” Curr. Opin. Genet. Dev., 3:74-80, 1993.
Samulski, Bems, Tan, Muzyczka, “Cloning of adeno-associated virus into pBR322: rescue of intact virus from the recombinant plasmid in human cells,” Proc. Natl. Acad. Sci. USA, 79(6):2077-2080, 1982.
Samulski, Srivastava, Bems, Muzyczka, “Rescue of adeno-associated virus from recombinant plasmids: gene correction within the terminal repeats of AAV,” Cell, 33(1):135-143, 1983.
Samulski, Chang and Shenk, “Helper-free stocks of recombinant adeno-associated viruses: normal integration does not require viral gene expression,” J. Virol., 63:3822-28, 1989.
Samulski, Zhu, Xiao, Brook, Housman, Epstein and Hunter, “Targeted integration of adeno-associated virus (AAV) into human chromosome 19,” Embo. J., 10:3941-50, (published erratum appears in Embo. J., 11:1228, 1992) 1991.
Sarkar, Legradi and Lechan, “Intracerebroventricular administration of alpha-melanocyte stimulating hormone increases phosphorylation of CREB in TRH- and CRH-producing neurons of the hypothalamic paraventricular nucleus,” Brain Res., 945:50-59, 2002.
Sarver, Cantin, Chang, Zaia, Ladne, Stephens, Rossi, “Ribozymes as a potential anti-HIV-1 therapeutic agents,” Science, 247(4947):1222-1225, 1990.
Satake, Furukawa and Ito, “Biological activities of oligonucleotides spanning the F9 point mutation within the enhancer region of polyoma virus DNA,” J. Virology, 62:970, 1988.
Satoh, Ogawa, Katsuura, Numata, Masuzaki, Yoshimasa and Nakao, “Satiety effect and sympathetic activation of leptin are mediated by hypothalamic melanocortin system,” Neurosci. Lett., 249:107-10, 1998.
Saville and Collins, “A site-specific self-cleavage reaction performed by a novel RNA in Neurospora mitochondria,” Cell, 61(4):685-696, 1990.
Saville and Collins, “RNA-mediated ligation of self-cleavage products of a Neurospora mitochondrial plasmid transcript,” Proc. Natl. Acad. Sci. USA, 88(19):8826-8830, 1991.
Scanlon, Jiao, Funato, Wang, Tone, Rossi, Kashani-Sabet, “Ribozyme-mediated cleavage of c-fos mRNA reduces gene expression of DNA synthesis enzymes and metallothionein,” Proc. Natl. Acad. Sci. USA, 88(23):10591-10595, 1991.
Scaringe, Francklyn, Usman, “Chemical synthesis of biologically active oligoribonucleotides using beta-cyanoethyl protected ribonucleoside phosphoramidites,” Nucl. Acids Res., 18(18):5433-5441, 1990.
Scarpace, Matheny, Pollock and Tumer, “Leptin increases uncoupling protein expression and energy expenditure,” Am. J. Physiol., 273:E226-30, 1997.
Scarpace, Matheny, Zhang, Turner, Frase, Shek, Hong, Prima and Zolotukhin, “Central leptin gene delivery evokes persistent leptin signal transduction in young and aged-obese rats but physiological responses become attenuated over time in aged-obese rats,” Neuropharmacology, 42:548-61, 2002a.
Scarpace, Matheny, Zhang, Shek, Prima, Zolotukhin and Turner, “Leptin-induced leptin resistance reveals separate roles for the anorexic and thermogenic responses in weight maintenance,” Endocrinology, 143:3026-35, 2002b
Schaffner, Schirm, Muller-Baden, Wever and Schaffner, “Redundancy of information in enhancers as a principle of mammalian transcription control,” J. Mol. Biol., 201:81, 1988.
Schwartz, Seeley, Campfield, Burn and Baskin, “Identification of targets of leptin action in rat hypothalamus,” J. Clin. Invest., 98:1101-06, 1996.
Schwartz, Seeley, Woods, Weigle, Campfield, Burn and Baskin, “Leptin increases hypothalamic pro-opiomelanocortin mRNA expression in the rostral arcuate nucleus,” Diabetes, 46:2119-23, 1997.
Sculier et al., “Pilot study of amphotericin B entrapped in sonicated liposomes in cancer patients with fungal infections,” J. Cancer Clin. Oncol., 24(3):527-538, 1988.
Searle, Stuart and Palmiter, “Building a metal-responsive promoter with synthetic regulatory elements,” Mol. Cell. Biol., 5:1480, 1985.
Seeley, Yagaloff, Fisher, Burn, Thiele, van Dijk, Baskin and Schwartz, “Melanocortin receptors in leptin effects,” Nature, 390:349, 1997.
Segal, “Biochemical Calculations” 2nd Edition. John Wiley & Sons, New York, 1976.
Senapathy, Tratschin and Carter, “Replication of adeno-associated virus DNA. Complementation of naturally occurring rep⁻ mutants by a wild-type genome or an ori⁻ mutant and correction of terminal palindrome deletions,” J. Mol. Biol., 179:1-20, 1984.
Shafron, Simpkins, Jebelli, Day and Meyer, “Reduced MK801 binding in neocortical neurons after AAV-mediated transfections with NMDA-R1 antisense cDNA,” Brain Res. 784:325-328, 1998.
Sharp and Marciniak, “HIV Tar: An RNA enhancer?” Cell, 59:229, 1989.
Shaul and Ben-Levy, “Multiple nuclear proteins in liver cells are bound to Hepatitis B virus enhancer element and its upstream sequences,” EMBO J., 6:1913, 1987.
Sherman, Basta, Moore, Brown and Ting, “Class II box consensus sequences in the HLA-DRa gene: Transcriptional function and interaction with nuclear proteins,” Mol. Cell. Biol., 9:50, 1989.
Shinyama, Masuzaki, Fang and Flier, “Regulation of melanocortin-4 receptor signaling: agonist-mediated desensitization and internalization,” Endocrinology, 144:1301-14, 2003.
Sleigh and Lockett, “SV40 enhancer activation during retinoic-acid-induced differentiation of F9 embryonal carcinoma cells,” J. EMBO, 4:3831, 1985.
Snyder, Spratt, Lagarde, Bohl, Kaspar, Sloan, Cohen and Danos, “Efficient and stable adeno-associated virus-mediated transduction in the skeletal muscle of adult immunocompetent mice,” Hum. Gene Ther., 8:1891-900, 1997a.
Snyder, Miao, Patijn, Spratt, Danos, Nagy, Gown, Winther, Meuse, Cohen, Thompson and Kay, “Persistent and therapeutic concentrations of human factor IX in mice after hepatic gene transfer of recombinant AAV vectors,” Nat. Genet., 16:270-76, 1997b.
Song, Morgan, Ellis, Poirer, Chesnut, Wang, Brantly, Muzyczka, Byrne, Atkinson and Flotte, “Sustained secretion of human α1-antitrypsin from murine muscle transduced with adeno-associated virus vectors,” Proc. Natl. Acad. Sci. USA, 95:14384-88, 1998.
Song, Embury, Laipis, Berns, Crawford and Flotte, “Stable therapeutic serum levels of human alpha-1 antitrypsin (AAT) after portal vein injection of recombinant adeno-associated virus (rAAV) vectors,” Gene Ther., 8:1299-306, 2001 a.
Song, Laipis, Berns and Flotte, “Effect of DNA-dependent protein kinase on the molecular fate of the rAAV2 genome in skeletal muscle,” Proc. Natl. Acad. Sci. USA, 98:4084-88, 2001b.
Spalholz, Yang and Howley, “Transactivation of a bovine papilloma virus transcriptional regulatory element by the E2 gene product,” Cell, 42:183, 1985.
Spandau and Lee, “Trans-activation of viral enhancers by the Hepatitis B virus X protein,” J. Virology, 62:427, 1988.
Spandidos and Wilkie, “Host-specificities of papilloma virus, Moloney murine sarcoma virus and simian virus 40 enhancer sequences,” EMBO J., 2:1193, 1983.
Srivastava, Lusby, Berns, “Nucleotide sequence and organization of the adeno-associated virus 2 genome,” J. Virol., 45(2):555-564, 1983.
Stephens and Hentschel, “The bovine papilloma virus genome and its uses as a eukaryotic vector,” Biochem. J., 248:1, 1987.
Stephenson and Gibson, Antisense Res. Dev., 1:261-68, 1991.
Stuart, Searle and Palmiter, “Identification of multiple metal regulatory elements in mouse metallothionein-I promoter by assaying synthetic sequences,” Nature, 317:828, 1985.
Sullivan and Peterlin, “Transcriptional enhancers in the HLA-DQ subregion,” Mol. Cell. Biol., 7:3315, 1987.
Summerford and Samulski, “Membrane-associated heparan sulfate proteoglycan is a receptor for adeno-associated virus type 2 virions,” J. Virol., 72:1438-45, 1998.
Summerford, Bartlett and Samulski, “αᵥβ₅ integrin: a co-receptor for adeno-associated virus type 2 infection,” Nat. Med., 5:78-82, 1999.
Suzuki, Shin, Fjuikura, Matsuzaki, Takata, “Direct gene transfer into rat liver cells by in vivo electroporation,” FEBS Lett., 425(3):436-440, 1998.
Swartzendruber and Lehman, “Neoplastic differentiation: Interaction of simian virus 40 and polyoma virus with murine teratocarcinoma cells,” J. Cell. Physiology, 85:179, 1975.
Taira, Nakagawa, Nishikawa, Furukawa, “Construction of a novel RNA-transcript-trimming plasmid which can be used both in vitro in place of run-off and (G)-free transcriptions and in vivo as multi-sequences transcription vectors,” Nuc. Acids Res., 19(19):5125-5130, 1991.
Takakura, “Drug delivery systems in gene therapy,” Nippon Rinsho, 56(3):691-695, 1998.
Takebe, Seiki, Fujisawa, Hoy, Yokota, Arai, Yoshida and Arai, “SRa promoter: An efficient and versatile mammalian cDNA expression system composed of the simian virus 40 early promoter and the R-U5 segment of human T-cell leukemia virus Type 1 long terminal repeat,” Mol. Cell. Biol., 8:466, 1988.
Tavernier, Gheysen, Duerinck, Can der heyden and fiers, “Deletion mapping of the inducible promoter of human IFN-β gene,” Nature, 301:634, 1983.
Taylor and Kingston, “Factor substitution in a human HSP70 gene promoter: TATA-dependent and TATA-independent interactions,” Mol. Cell. Biol., 10:165, 1990a.
Taylor and Kingston, “Ela trans-activation of human HSP70 gene promoter substitution mutants is independent of the composition of upstream and TATA elements,” Mol. Cell. Biol., 10:176, 1990b.
Taylor and Rossi, Antisense Res. Dev., 1: 173-86, 1991.
Taylor, Solomon, Weiner, Paucha, Bradley and Kingston, “Stimulation of the human heat-shock protein 70 promoter in vitro by simian virus 40 large T antigen,” J. Biol. Chem., 264:15160, 1989.
Thiesen, Bosze, Henry and Chamay, “A DNA element responsible for the different tissue specificities of friend and Moloney retroviral enhancers,” J. Virology, 62:614, 1988.
Tratschin, Miller, Carter, “Genetic analysis of adeno-associated virus: properties of deletion mutants constructed in vitro and evidence for an adeno-associated virus replication finction,” J. Virol., 51(3):611-619, 1984a.
Tratschin, West, Sandbank, Carter, “A human parvovirus, adeno-associated virus, as a eucaryotic vector: transient expression and encapsidation of the procaryotic gene for chloramphenicol acetyltransferase,” Mol. Cell. Biol., 4(10):2072-2081, 1984b.
Treisman, “identification of a protein-binding site that mediates transcriptional response to the c-fos gene to serum factors,” Cell, 46(4):567-574, 1986.
Trempe and Carter, “Alternate mRNA splicing is required for synthesis of adeno-associated virus VP1 capsid protein,” J. Virol., 62:3356-63, 1988.
Tronche, Rollier, Bach, Weiss and Yaniv, “The rat albumin promoter: Cooperation with upstream elements is required when binding of APF/HNF 1 to the proximal element is partially impaired by mutation or bacterial methylation,” Mol. Cell. Biol., 9:4759, 1989.
Tronche, Rollier, Herbomel, Bach, Cereghini, Weiss and Yaniv, “Anatomy of the rat albumin promoter,” Mol. Biol. Med., 7:173, 1990.
Trudel and Constantini, “A 3′ enhancer contributes to the stage-specific expression of the human β-globin gene,” Genes and Dev., 6:954, 1987.
Tur-Kaspa et al., “Use of electroporation to introduce biologically active foreign genes into primary rat hepatocytes,” Mol. Cell Biol., 6:716-718, 1986.
Tyndall, La Mantia, Thacker, Favaloro and Kamen, “A region of the polyoma virus genome between the replication origin and late protein-coding sequences is required in cis for both early gene expression and viral DNA replication,” Nuc. Acids. Res., 9:6231, 1981.
Uhler and Herbert, “Complete amino acid sequence of mouse pro-opiomelanocortin derived from the nucleotide sequence of pro-opiomelanocortin cDNA,” J. Biol. Chem., 258:257-61, 1983.
Usman and Cedergren, “Exploiting the chemical synthesis of RNA,” Trends Biochem. Sci., 17(9):334, 1992.
Usman et al., J. Am. Chem. Soc., 109:7845-7854, 1987.
Van Cott, Lubon, Russell, Butler, Gwazdauskas, Knight, Drohan, Velander, “Phenotypic and genotypic stability of multiple lines of transgenic pigs expressing recombinant human protein C,” Transgenic Res., 6(3):203-212, 1997.
Vanbever, Fouchard, Jadoul, De Morre, Preat, Marty, “In vivo noninvasive evaluation of hairless rat skin after high-voltage pulse exposure,” Skin Parmacol. Appl. Skin Physiol., 11(1):23-34, 1998.
Vannice and Levinson, “Properties of the human Hepatitis B virus enhancer: Position effects and cell-type nonspecificity,” J. Virology, 62:1305, 1988.
Vasseur, Kress, Montreau and Blangy, “Isolation and characterization of polyoma virus mutants able to develop in multipotential murine embryonal carcinoma cells,” Proc. Natl. Acad. Sci. US.A., 77:1068, 1980.
Ventura, Wang, Ragot, Perricaudet, Saragosti, “Activation of HIV-specific ribozyme activity by self-cleavage,” Nucl. Acids Res., 21:3249-3255, 1993.
Virella-Lowell, Song, Morgan and Flotte, “A CMV/β-actin hybrid promoter greatly improves recombinant adeno-associated virus (rAAV) vector expression in the murine lung,” Ped. Pulmonol., S19:231, 1999.
von-Weizsacker, Blum and Wands, Biochem. Biophys. Res. Commun., 189:743-48, 1992.
Wagner, Zatloukal, Cotten, Kirlappos, Mechtler, Curiel, Birnstiel, “Coupling of adenovirus to transferrin-polylysine/DNA complexes greatly enhances receptor-mediated gene delivery and expression of transfected genes,” Proc. Natl. Acad. Sci. USA, 89(13):6099-6103, 1992.
Wagner, Reynolds, Moran, Moss, Wine, Flotte and Gardner, “Efficient and persistent gene transfer of AAV-CFTR in maxillary sinus,” Lancet, 351:1702-03, 1998.
Walker, et al., “Strand displacement amplification—an isothermal, in vitro DNA amplification technique,” Nucleic Acids Res., 20(7):1691-6, 1992.
Wang and Calame, “SV40 enhancer-binding factors are required at the establishment but not the maintenance step of enhancer-dependent transcriptional activation,” Cell, 47:241, 1986.
Wang, et al., “NGF gene expression in dividing and non-dividing cells from AAV-derived constructs,” Neurochem Res., 23(5):779-86, 1998.
Weber, de Villiers and Schaffner, “An SV40 ‘enhancer trap’ incorporates exogenous enhancers or generates enhancers from its own sequences,” Cell, 36:983, 1984.
Weinberger, Jat and Sharp, “Localization of a repressive sequence contributing to B-cell specificity in the immunoglobulin heavy-chain enhancer,” Mol. Cell. Biol., 8:988, 1984.
Weitzman, Kyostio, Kotin and Owens, “Adeno-associated virus (AAV) Rep proteins mediate complex formation between AAV DNA and its integration site in human DNA,” Proc. Natl. Acad. Sci. USA, 91:5808-12, 1994.
Winoto and Baltimore, “αβ-lineage-specific expression of the a T-cell receptor gene by nearby silencers,” Cell, 59:649, 1989.
Woolf, Melton, Jennings, “Specificity of antisense oligonucleotides in vivo,” Proc. Natl. Acad. Sci. USA, 89(16):7305-7309, 1992.
Wong and Neumann, “Electric field mediated gene transfer,” Biochim. Biophys. Res. Commun., 107(2):584-587, 1982.
Wu and Dean, “Functional significance of loops in the receptor binding domain of Bacillus thuringiensis CryIIIA delta-endotoxin,” J. Mol. Biol., 255(4):628-640, 1996.
Wu and Wu, “Evidence for targeted gene delivery to HepG2 hepatoma cells in vitro,” Biochemistry, 27:887-892, 1988.
Wu and Wu, “Receptor-mediated in vitro gene transfections by a soluble DNA carrier system,” J. Biol. Chem., 262:4429-4432, 1987.
Wu, Xiao, Conlon, Hughes, Agbandje-McKenna, Ferkol, Flotte and Muzyczka, “Mutational analysis of the adeno-associated virus type 2 (AAV2) capsid gene and construction of AAV2 vectors with altered tropism,” J Virol., 74:8635-47, 2000.
Xiao, Li and Samulski, “Efficient long-term gene transfer into muscle tissue of immuno-competent mice by adeno-associated virus vector,” J. Virol., 70:8098-108, 1996.
Xiao, Berta, Lu, Moscioni, Tazelaar and Wilson, “Adeno-associated virus as a vector for liver-directed gene therapy,” J. Virol., 72:10222-26, 1998.
Xing and Whitton, J. Virol., 67:1840-47, 1993.
Xu, Janson, Mastakov, Lawlor, Young, Mouravlev, Fitzsimons, Choi, Ma, Dragunow, Leone, Chen, Dicker and During, “Quantitative comparison of expression with adeno-associated virus (AAV- 2) brain-specific gene cassettes,” Gene Ther., 8:1323-32, 2001.
Yan, Zhang, Duan and Engelhardt, “From the cover: trans-splicing vectors expand the utility of adeno-associated virus for gene therapy,” Proc. Natl. Acad. Sci. USA, 97:6716-21, 2000.
Yang et al., “In vivo and in vitro gene transfer to mammalian somatic cells by particle bombardment,” Proc. Natl. Acad. Sci. USA, 87:9568-9572, 1990.
Yaswen, Diehl, Brennan and Hochgeschwender, “Obesity in the mouse model of pro-opiomelanocortin deficiency responds to peripheral melanocortin,” Nat. Med., 5:1066-70, 1999.
Yu, Ojwang, Yamada, Hampel, Rapapport, Looney, Wong-Staal, “A hairpin ribozyme inhibits expression of diverse strains of human immunodeficiency virus type 1,” Proc. Natl. Acad. Sci. USA, 90:6340-6344, 1993.
Yutzey, Kline and Konieczny, “An internal regulatory element controls troponin I gene expression,” Mol. Cell. Biol., 9:1397, 1989.
Zabner, Seiler, Walters, Kotin, Fulgeras, Davidson and Chiorini, “Adeno-associated virus type 5 (AAV5) but not AAV2 binds to the apical surfaces of airway epithelia and facilitates gene transfer,” J. Virol., 74:3852-58, 2000.
Zambaux, Bonneaux, Gref, Maincent, Dellacherie, Alonso, Labrude, Vigneron, “Influence of experimental paparmeters on the characteristics of poly(lactic acid) nanoparticles prepared by a double emulsion method,” J. Controlled Release, 50(1-3):31-40, 1998.
Zhang, Matheny, Tumer and Scarpace, “Aged-obese rats exhibit robust responses to a melanocortin agonist and antagonist despite leptin resistance,” Neurobiology of Aging, In press, 2004.
Zhou, Giordano, Durbin, McAllister, “Synthesis of functional mRNA in mammalian cells by bacteriophage T3 RNA polymerase,” Mol. Cell Biol., 10(9):4529-4537, 1990.
Zolotukhin, Potter, Hauswirth, Guy and Muzyczka, “A ‘humanized’ green fluorescent protein cDNA adapted for high-level expression in mammalian cells,” J. Virol., 70:4646-54, 1996.
Zolotukhin, Byrne, Mason, Zolotukhin, Potter, Chesnut, Summerford, Samulski and Muzyczka, “Recombinant adeno-associated virus purification using novel methods improves infectious titer and yield,” Gene Ther., 6:973-85, 1999.
Zolotukhin, Potter, Zolotukhin, Sakai, Loiler, Fraites Jr., Chiodo, Phillipsberg, Muzyczka, Hauswirth, Flotte, Byrne and Snyder, “Production and purification of serotype 1, 2, and 5 recombinant adeno-associated viral vectors,” Methods, 28:158-67, 2002.
zur Muhlen, Schwarz, Mehnert, “Solid lipid nanoparticles (SLN) for controlled drug delivery--drug release and release mechanism,” Eur. J. Pharm. Biopharm., 45(2):149-155, 1998.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition comprising an adeno-associated viral vector that comprises a nucleic acid segment encoding a pro-opiomelanocortin polypeptide operably linked to a promoter capable of expressing said segment in a host cell that comprises said vector, wherein said polypeptide activates the central melanocortin pathway in a mammal that expresses said vector.

2. The composition of claim 1, wherein said vector further comprises an enhancer sequence operably linked to said nucleic acid segment.

3. The composition of claim 1, wherein said vector further comprises a post-transcriptional regulatory element operably linked to said nucleic acid segment.

4. The composition of claim 1, wherein said nucleic acid segment encodes a mammalian pro-opiomelanocortin polypeptide.

5. The composition of claim 1, wherein said promoter is an inducible promoter.

6. The composition of claim 1, further comprising a pharmaceutically-acceptable excipient.

7. The composition of claim 1, wherein said vector is comprised within an rAAV virion.

8. The composition of claim 7, further comprising a liposome, a lipid, or a lipid complex.

9. The composition of claim 7, further comprising a microsphere or a nanoparticle.

10. The composition of claim 7, formulated for administration to a human.

11. The composition of claim 1, comprised within a kit for diagnosing, preventing, treating or ameliorating the symptoms of a pro-opiomelanocortin polypeptide deficiency condition in a mammal.

12. A recombinant adeno-associated viral particle comprising an adeno-associated viral vector that comprises a nucleic acid segment encoding a pro-opiomelanocortin polypeptide operably linked to a promoter capable of expressing said segment in a host cell that comprises said vector, wherein said polypeptide activates the central melanocortin pathway in a mammal that expresses said vector.

13. A method for treating or ameliorating the symptoms of a pro-opiomelanocortin polypeptide deficiency condition in a mammal, said method comprising administering to said mammal the composition of claim 1; in an amount and for a time sufficient to treat or ameliorate the symptoms of said deficiency in said mammal.

14. The method of claim 13, wherein said deficiency condition results in polyphagia, hyperinsulinemia, adiposity, an eating disorder, or body weight gain in said mammal.

15. The method of claim 13, wherein said composition is administered to said mammal in an amount and for a time sufficient to decrease the body weight of said mammal, or to decrease the rate of body weight gain in said mammal.

16. The method of claim 13, wherein said composition is administered to said mammal intramuscularly, intravenously, intrathecally, or intracerebroventricularly.

17. A method for providing a mammal in need thereof with a therapeutically-effective amount of a pro-opiomelanocortin polypeptide, said method comprising introducing into suitable cells or a tissue of said mammal, an amount of the composition of claim 1; for a time effective to provide said mammal with a therapeutically-effective amount of said pro-opiomelanocortin polypeptide.

18. The method of claim 17, wherein said composition is introduced into said cells or said tissue ex vivo; and further wherein said method comprises the additional step of introducing the resulting cells or tissue that comprise said composition into said mammal.

19. The method of claim 17, wherein said mammal has been diagnosed with obesity, adiposity, or suffers from excessive body weight gain.

20. A method for controlling body weight gain or food intake in a mammal, said method comprising at least the step of introducing into a cell or tissue of the brain of said mammal, a therapeutically-effective amount of the composition of claim 1, for a time effective to control said body weight gain or said food intake in said mammal.
